SEQUENCE LISTING (211 sequences)

SEQ ID NO: 1 - protein, 114 aa, Artificial Sequence
  IL-15
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 77 aa, Artificial Sequence
  IL-15-Ralphasushi
ITCPPPMSVEHADIWVKSYSLYSRERYICNSGFKRKAGTSSLTECVLNKATNVAHWTTPSLKCIRDPALVHQRPAPP

SEQ ID NO: 3 - DNA, 912 bp, Artificial Sequence
  IL-15-Ralphasushi_GS linker_BLV1H12 light chain
atcacctgcccacctccaatgagcgtggagcacgcagacatctgggtgaagtcttacagcctgtattcccgggagagatacatctgcaactctggcttcaagcggaaggccggcaccagctccctgacagagtgcgtgctgaacaaggccaccaatgtggcccactggacaactccttccctgaaatgtattagagaccccgccctggtgcatcagagacctgccccccctggtggaggcggttcaggcggaggtggatcccaggccgtcctgaaccagccaagcagcgtctccgggtctctggggcagcgggtctcaatcacctgtagcgggtcttcctccaatgtcggcaacggctacgtgtcttggtatcagctgatccctggcagtgccccacgaaccctgatctacggcgacacatccagagcttctggggtccccgatcggttctcagggagcagatccggaaacacagctactctgaccatcagctccctgcaggctgaggacgaagcagattatttctgcgcatctgccgaggactctagttcaaatgccgtgtttggaagcggcaccacactgacagtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataa

SEQ ID NO: 4 - protein, 524 aa, Artificial Sequence
  IL2 receptor subunit beta
VNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 5 - DNA, 513 bp, Artificial Sequence
  BLV1H 12 heavy chain
caggtccagctgagagagagcggcccttcactggtcaagccatcccagacactgagcctgacatgcacagcaagcgggttttcactgagcgacaaggcagtgggatgggtccgacaggcaccaggaaaagccctggaatggctgggcagcatcgataccggcgggaacacagggtacaatcccggactgaagagcagactgtccattaccaaggacaactctaaaagtcaggtgtcactgagcgtgagctccgtcaccacagaggatagtgcaacttactattgcacctctgtgcaccaggaaactaagaaataccagagctgtcctgacggctatcgggagagatctgattgcagtaataggccagcttgtggcacatccgactgctgtcgcgtgtctgtcttcgggaactgcctgactaccctgcctgtgtcctactcttatacctacaattatgaatggcatgtggatgtctggggacagggcctgctggtgacagtctctagtgctagc

SEQ ID NO: 6 - DNA, 1557 bp, Artificial Sequence
  (Sig Seq- VRegion - CH1CH2CH3) BLV1H12 V in human IgG
atgggatggtcatgtatcatcctttttctagtagcaactgcaaccggtgtacattcccaggtgcagctgcgggagtcgggccccagcctgatgaagccgtcacagaccctctccctcacctgcacggtctctggatcttcattgaacgacaagtctgtaggctgggtccgccaggctccagggaaggcgctgcagtggctcggtagtgtggacactagtggaaacacagactataacccaggcctgaaatcccggctcagcatcaccaaggacaactccaagagccgaatctctcttacagtgactggcatgacaactgaagactcggccacatactactgtacttctgtgcaccaggaaacaaaaaaataccaaagttgtccggaggattatacttataatccacgttgccctcagcagtatggttggagtgactgtgattgtatgggcgataggtttgggggttactgtcgacaggatggttgtagtaattatagttatacttacaattacgaatggcacgtcgatgtctggggccaaggactcctggtcaccgtctcctcagctagcaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 7 - DNA, 823 bp, Artificial Sequence
  B15 variable region plus signal peptide
gaattccaccatgggatggtcatgtatcatcctttttctagtagcaactgcaaccggagtacattcccaggtgcagctgcgcgagtcgggccccagcctggtgaagccgtcacagaccctctcgctcacctgcacggcctctggattctcattgagcgacaaggctgtaggctgggtccgccaggctccagggaaggcgctggagtggctcggtagtatagacactggtggaaacacaggctataacccaggcctgaaatcccggctcagcatcaccaaggacaactccaagagtcaagtctctctgtcagtgagcagcgtgacaactgaggactcggccacatactactgtacttctgtgcaccaggaaacaaaaaaataccaaaccggtggatcaaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctggttcaggatcctatacttacaattacgaatggcacgtcgatgtctggggccaaggactcctggtcaccgtctcctcagctagc

SEQ ID NO: 8 - DNA, 672 bp, Artificial Sequence
  BLV1H12 Light Chain
tcacgaattcgcaggccgtcctgaaccagccaagcagcgtctccgggtctctggggcagcgggtctcaatcacctgtagcgggtcttcctccaatgtcggcaacggctacgtgtcttggtatcagctgatccctggcagtgccccacgaaccctgatctacggcgacacatccagagcttctggggtccccgatcggttctcagggagcagatccggaaacacagctactctgaccatcagctccctgcaggctgaggacgaagcagattatttctgcgcatctgccgaggactctagttcaaatgccgtgtttggaagcggcaccacactgacagtcctggggcagcccaagagtcccccttcagtgactctgttcccaccctctaccgaggaactgaacggaaacaaggccacactggtgtgtctgatcagcgacttttaccctggatccgtcactgtggtctggaaggcagatggcagcacaattactaggaacgtggaaactacccgcgcctccaagcagtctaatagtaaatacgccgccagctcctatctgagcctgacctctagtgattggaagtccaaagggtcatatagctgcgaagtgacccatgaaggctcaaccgtgactaagactgtgaaaccatccgagtgctcctaggctagctggc

SEQ ID NO: 9 - protein, 12 aa, Artificial Sequence
  BLV1H12 ascending stalk region
TSVHQETKKYQS

SEQ ID NO: 10 - protein, 12 aa, Artificial Sequence
  BLV1H12 decending stalk region
SYTYNYEWHVDV

SEQ ID NO: 11 - protein, 11 aa, Artificial Sequence
  V2 alternative sequence
WGQGLLVTVSS

SEQ ID NO: 12 - protein, 95 aa, Artificial Sequence
  V1 Alternative B sequence of VH4-34_Q5RQ6E
QVQLREWGAGLLKPSETLSLTCAVYGGSFSGYYWSWIRQPPGKGLEWIGEINHSGSTNYNPSLKSRVTISVDTSKNQFSLKLSSVTAADTAVYYC

SEQ ID NO: 13 - protein, 95 aa, Artificial Sequence
  V1 Alternative B sequence of VH4-34_CDR1-G31DY32K_Q5RQ6E
QVQLREWGAGLLKPSETLSLTCAVYGGSFSDKYWSWIRQPPGKGLEWIGEINHSGSTNYNPSLKSRVTISVDTSKNQFSLKLSSVTAADTAVYYC

SEQ ID NO: 14 - protein, 95 aa, Artificial Sequence
  V1 Alternative B sequence of VH4-34_CDR2-E50S_Q5RQ6E
QVQLREWGAGLLKPSETLSLTCAVYGGSFSGYYWSWIRQPPGKGLEWIGSINHSGSTNYNPSLKSRVTISVDTSKNQFSLKLSSVTAADTAVYYC

SEQ ID NO: 15 - protein, 95 aa, Artificial Sequence
  synthesized V1 Alternative B sequence of VH4-34_CDR1-G31DY32K_CDR2-E50S_Q5RQ6E
QVQLREWGAGLLKPSETLSLTCAVYGGSFSDKYWSWIRQPPGKGLEWIGSINHSGSTNYNPSLKSRVTISVDTSKNQFSLKLSSVTAADTAVYYC

SEQ ID NO: 16 - protein, 95 aa, Artificial Sequence
  synthesized V1 Alternative B sequence of VH4-34_CDR1-Cow_Q5RQ6E
QVQLREWGAGLLKPSETLSLTCTASGFSLSDKAVGWIRQPPGKGLEWIGEINHSGSTNYNPSLKSRVTISVDTSKNQFSLKLSSVTAADTAVYYC

SEQ ID NO: 17 - protein, 95 aa, Artificial Sequence
  synthesized V1 Alternative B sequence of VH4-34_CDR2-Cow_Q5RQ6E
QVQLREWGAGLLKPSETLSLTCAVYGGLGSIDTGGNTGSFSGYYWSWIRQPPGKGLEWYNPSLKSRVTISVDTSKNQFSLKLSSVTAADTAVYYC

SEQ ID NO: 18 - protein, 95 aa, Artificial Sequence
  synthesized V1 Alternative B sequence of VH4-34_CDR1-Cow_CDR2-E50S_Q5RQ6E
QVQLREWGAGLLKPSETLSLTCTASGFSLSDKAVGWIRQPPGKGLEWIGSINHSGSTNYNPSLKSRVTISVDTSKNQFSLKLSSVTAADTAVYYC

SEQ ID NO: 19 - protein, 95 aa, Artificial Sequence
  synthesized V1 Alternative N sequence of VH4-34_CDR1-Cow_CDR2-Cow_Q5RQ6E
QVQLREWGAGLLKPSETLSLTCTASGFSLSDKAVGWIRQPPGKGLEWLGSIDTGGNTGYNPSLKSRVTISVDTSKNQFSLKLSSVTAADTAVYYC

SEQ ID NO: 20 - protein, 11 aa, Artificial Sequence
  V2 alternative sequence
WGHGTAVTVSS

SEQ ID NO: 21 - protein, 11 aa, Artificial Sequence
  V2 alternative sequence
WGKGTTVTVSS

SEQ ID NO: 22 - protein, 11 aa, Artificial Sequence
  V2 alternative sequence
WGKGTTVTVSS

SEQ ID NO: 23 - protein, 11 aa, Artificial Sequence
  V2 alternative sequence
WGRGTLVTVSS

SEQ ID NO: 24 - protein, 11 aa, Artificial Sequence
  V2 alternative sequence
WGKGTTVTVSS

SEQ ID NO: 25 - protein, 61 aa, Artificial Sequence
  Synthesized ultralong CDR3 sequence (BLV1H12)
SVHQETKKYQSCPDGYRERSDCSNRPACGTSDCCRVSVFGNCLTTLPVSYSYTYNYEWHVD

SEQ ID NO: 26 - protein, 274 aa, Artificial Sequence
  BLV1H12 Heavy Chain
QVQLRESGPSLVKPSQTLSLTCTASGFSLSDKAVGWVRQAPGKALEWLGSIDTGGNTGYNPGLKSRLSITKDNSKSQVSLSVSSVTTEDSATYYCTSVHQETKKYQSCPDGYRERSDCSNRPACGTSDCCRVSVFGNCLTTLPVSYSYTYNYEWHVDVWGQGLLVTVSSASTTAPKVYPLSSCCGDKSSSTVTLGCLVSSYMPEPVTVTWNSGALKSGVHTFPAVLQSSGLYSLSSMVTVPGSTSGQTFTCNVAHPASSTKVDKAVEPKSCDGS

SEQ ID NO: 27 - protein, 216 aa, Artificial Sequence
  BLV1H12 Light Chain
QAVLNQPSSVSGSLGQRVSITCSGSSSNVGNGYVSWYQLIPGSAPRTLIYGDTSRASGVPDRFSGSRSGNTATLTISSLQAEDEADYFCASAEDSSSNAVFGSGTTLTVLGQPKSPPSVTLFPPSTEELNGNKATLVCLISDFYPGSVTVVWKADGSTITRNVETTRASKQSNSKYAASSYLSLTSSDWKSKGSYSCEVTHEGSTVTKTVKPSECS

SEQ ID NO: 28 - protein, 240 aa, Artificial Sequence
  BLV5B8 heavy chain
QVQLRESGPSLVQPSQTLSLTCTASGFSLSDKAVGWVRQAPGKALEWLGSIDTGGSTGYNPGLKSRLSITKDNSKSQVSLSVSSVTTEDSATYYCTTVHQETRKTCSDGYIAVDSCGRGQSDGCVNDCNSCYYGWRNCRRQPAIHSYEFHVDAWGRGLLVTVSSASTTAPKVYPLSSCCGDKSSSTVTLGCLVSSYMPEPVTVTWNSGALKSGVHTFPAVLQSSGLYSLSSMVTVPGSTS

SEQ ID NO: 29 - protein, 216 aa, Artificial Sequence; identical to SEQ ID NO: 27
  BLV5B8 light chain

SEQ ID NO: 30 - protein, 56 aa, Artificial Sequence
  BLV5B8 CDR3
TVHQETRKTCSDGYIAVDSCGRGQSDGCVNDCNSCYYGWRNCRRQPAIHSYEFHVD

SEQ ID NO: 31 - protein, 57 aa, Artificial Sequence
  BLV5D3 CDR3
SVTQRTHVSRSCPDGCSDGDGCVDGCCCSAYRCYTPGVRDLSCTSYSITYTYEWNVD

SEQ ID NO: 32 - protein, 58 aa, Artificial Sequence
  BLV8C11 CDR3
TVHQKTTRKTCCSDAYRYDSGCGSGCDCCGADCYVFGACTFGLDSSYSYIYIYQWYVD

SEQ ID NO: 33 - protein, 56 aa, Artificial Sequence
  BF4E9 CDR3
TVHQIFCPDGYSYGYGCGYGYGCSGYDCYGYGGYGYGGYGGYSSYSYSYSYEYYGD

SEQ ID NO: 34 - protein, 48 aa, Artificial Sequence
  BF1H1 CDR3
TVHPSPDGYSYGYGCGYGYGCSGYDCYGYGGYGYGGYGGYSSYSYSYS

SEQ ID NO: 35 - protein, 48 aa, Artificial Sequence
  F18 CDR3
TVHQIRCPDGYGYGYGCGYGSYGYSGYDCYGYGGYGGYGGYGGYSSYS

SEQ ID NO: 36 - protein, 5 aa, Artificial Sequence
  Ascending Stalk Strand
TTVHQ

SEQ ID NO: 37 - protein, 5 aa, Artificial Sequence
  Ascending Stalk Strand
TSVHQ

SEQ ID NO: 38 - protein, 5 aa, Artificial Sequence
  Ascending Stalk Strand
SSVTQ

SEQ ID NO: 39 - protein, 5 aa, Artificial Sequence
  Ascending Stalk Strand
STVHQ

SEQ ID NO: 40 - protein, 5 aa, Artificial Sequence
  Ascending Stalk Strand
ATVRQ

SEQ ID NO: 41 - protein, 5 aa, Artificial Sequence
  Ascending Stalk Strand
TTVYQ

SEQ ID NO: 42 - protein, 5 aa, Artificial Sequence
  Ascending Stalk Strand
SPVHQ

SEQ ID NO: 43 - protein, 5 aa, Artificial Sequence
  Ascending Stalk Strand
ATVYQ

SEQ ID NO: 44 - protein, 5 aa, Artificial Sequence
  Ascending Stalk Strand
TAVYQ

SEQ ID NO: 45 - protein, 5 aa, Artificial Sequence
  Ascending Stalk Strand
TNVHQ

SEQ ID NO: 46 - protein, 5 aa, Artificial Sequence
  Ascending Stalk Strand
ATVHQ

SEQ ID NO: 47 - protein, 5 aa, Artificial Sequence
  Ascending Stalk Strand
STVYQ

SEQ ID NO: 48 - protein, 5 aa, Artificial Sequence
  Ascending Stalk Strand
TIVHQ

SEQ ID NO: 49 - protein, 5 aa, Artificial Sequence
  Ascending Stalk Strand
AIVYQ

SEQ ID NO: 50 - protein, 5 aa, Artificial Sequence
  Ascending Stalk Strand
TTVFQ

SEQ ID NO: 51 - protein, 5 aa, Artificial Sequence
  Ascending Stalk Strand
AAVFQ

SEQ ID NO: 52 - protein, 5 aa, Artificial Sequence
  Ascending Stalk Strand
GTVHQ

SEQ ID NO: 53 - protein, 5 aa, Artificial Sequence
  Ascending Stalk Strand
ASVHQ

SEQ ID NO: 54 - protein, 5 aa, Artificial Sequence
  Ascending Stalk Strand
TAVFQ

SEQ ID NO: 55 - protein, 5 aa, Artificial Sequence
  Ascending Stalk Strand
ATVFQ

SEQ ID NO: 56 - protein, 5 aa, Artificial Sequence
  Ascending Stalk Strand
AAAHQ

SEQ ID NO: 57 - protein, 4 aa, Artificial Sequence
  Ascending Stalk Strand
VWYQ

SEQ ID NO: 58 - protein, 5 aa, Artificial Sequence
  Ascending Stalk Strand
GTVFQ

SEQ ID NO: 59 - protein, 5 aa, Artificial Sequence
  Ascending Stalk Strand
TAVHQ

SEQ ID NO: 60 - protein, 5 aa, Artificial Sequence
  Ascending Stalk Strand
ITVHQ

SEQ ID NO: 61 - protein, 5 aa, Artificial Sequence
  Ascending Stalk Strand
ITAHQ

SEQ ID NO: 62 - protein, 5 aa, Artificial Sequence
  Ascending Stalk Strand
VTVHQ

SEQ ID NO: 63 - protein, 5 aa, Artificial Sequence
  Ascending Stalk Strand
AAVHQ

SEQ ID NO: 64 - protein, 5 aa, Artificial Sequence
  Ascending Stalk Strand
GTVYQ

SEQ ID NO: 65 - protein, 5 aa, Artificial Sequence
  Ascending Stalk Strand
TTVLQ

SEQ ID NO: 66 - protein, 5 aa, Artificial Sequence
  Ascending Stalk Strand
TTTHQ

SEQ ID NO: 67 - protein, 5 aa, Artificial Sequence
  Ascending Stalk Strand
TTDYQ

SEQ ID NO: 68 - protein, 99 aa, Artificial Sequence
  Human heavy chain variable region sequence VH4-39
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAR

SEQ ID NO: 69 - protein, 96 aa, Artificial Sequence
  Human heavy chain variable region sequence 4-59*03
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCA

SEQ ID NO: 70 - protein, 97 aa, Artificial Sequence
  Human heavy chain variable region sequence 4-34*02
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAR

SEQ ID NO: 71 - protein, 97 aa, Artificial Sequence
  Human heavy chain variable region sequence 4-34*09
QVQLQESGPGLVKPSQTLSLTCAVYGGSFSGYYWSWIRQPPGKGLEWIGEINHSGSTNYNPSLKSRVTISVDTSKNQFSLKLSSVTAADTAVYYCAR

SEQ ID NO: 72 - protein, 11 aa, Artificial Sequence
  Ascending Stalk Strand
TSVHQETKKYQ

SEQ ID NO: 73 - protein, 9 aa, Artificial Sequence
  Ascending Stalk Strand
VHQETKKYQ

SEQ ID NO: 74 - protein, 6 aa, Artificial Sequence
  Ascending Stalk Strand
IHSYEF

SEQ ID NO: 75 - protein, 4 aa, Artificial Sequence
  Ascending Stalk Strand
SYEF

SEQ ID NO: 76 - protein, 6 aa, Artificial Sequence
  Descending Stalk Strand
YTYNYE

SEQ ID NO: 77 - protein, 7 aa, Artificial Sequence
  Descending Stalk Strand
YTYNYEW

SEQ ID NO: 78 - protein, 8 aa, Artificial Sequence
  Descending Stalk Strand
SYTYNYEW

SEQ ID NO: 79 - protein, 6 aa, Artificial Sequence
  Descending Stalk Strand
TYNYEW

SEQ ID NO: 80 - protein, 4 aa, Artificial Sequence
  Descending Stalk Strand
SYTY

SEQ ID NO: 81 - protein, 14 aa, Artificial Sequence
  Ascending Stalk Strand
GSKHRLRDYFLYNE

SEQ ID NO: 82 - protein, 13 aa, Artificial Sequence
  Ascending Stalk Strand
GSKHRLRDYFLYN

SEQ ID NO: 83 - protein, 12 aa, Artificial Sequence
  Ascending Stalk Strand
GSKHRLRDYFLY

SEQ ID NO: 84 - protein, 11 aa, Artificial Sequence
  Ascending Stalk Strand
GSKHRLRDYFL

SEQ ID NO: 85 - protein, 10 aa, Artificial Sequence
  Ascending Stalk Strand
GSKHRLRDYF

SEQ ID NO: 86 - protein, 9 aa, Artificial Sequence
  Ascending Stalk Strand
GSKHRLRDY

SEQ ID NO: 87 - protein, 8 aa, Artificial Sequence
  Ascending Stalk Strand
GSKHRLRD

SEQ ID NO: 88 - protein, 13 aa, Artificial Sequence
  Ascending Stalk Strand
EAGGPDYRNGYNY

SEQ ID NO: 89 - protein, 12 aa, Artificial Sequence
  Ascending Stalk Strand
EAGGPDYRNGYN

SEQ ID NO: 90 - protein, 11 aa, Artificial Sequence
  Ascending Stalk Strand
EAGGPDYRNGY

SEQ ID NO: 91 - protein, 10 aa, Artificial Sequence
  Ascending Stalk Strand
EAGGPDYRNG

SEQ ID NO: 92 - protein, 9 aa, Artificial Sequence
  Ascending Stalk Strand
EAGGPDYRN

SEQ ID NO: 93 - protein, 8 aa, Artificial Sequence
  Ascending Stalk Strand
EAGGPDYR

SEQ ID NO: 94 - protein, 7 aa, Artificial Sequence
  Ascending Stalk Strand
EAGGPDY

SEQ ID NO: 95 - protein, 6 aa, Artificial Sequence
  Ascending Stalk Strand
EAGGPD

SEQ ID NO: 96 - protein, 13 aa, Artificial Sequence
  Ascending Stalk Strand
EAGGPIWHDDVKY

SEQ ID NO: 97 - protein, 12 aa, Artificial Sequence
  Ascending Stalk Strand
EAGGPIWHDDVK

SEQ ID NO: 98 - protein, 11 aa, Artificial Sequence
  Ascending Stalk Strand
EAGGPIWHDDV

SEQ ID NO: 99 - protein, 10 aa, Artificial Sequence
  Ascending Stalk Strand
EAGGPIWHDD

SEQ ID NO: 100 - protein, 9 aa, Artificial Sequence
  Ascending Stalk Strand
EAGGPIWHD

SEQ ID NO: 101 - protein, 8 aa, Artificial Sequence
  Ascending Stalk Strand
EAGGPIWH

SEQ ID NO: 102 - protein, 7 aa, Artificial Sequence
  Ascending Stalk Strand
EAGGPIW

SEQ ID NO: 103 - protein, 6 aa, Artificial Sequence
  Ascending Stalk Strand
EAGGPI

SEQ ID NO: 104 - protein, 11 aa, Artificial Sequence
  Ascending Stalk Strand
GTDYTIDDQGI

SEQ ID NO: 105 - protein, 10 aa, Artificial Sequence
  Ascending Stalk Strand
GTDYTIDDQG

SEQ ID NO: 106 - protein, 9 aa, Artificial Sequence
  Ascending Stalk Strand
GTDYTIDDQ

SEQ ID NO: 107 - protein, 8 aa, Artificial Sequence
  Ascending Stalk Strand
GTDYTIDD

SEQ ID NO: 108 - protein, 7 aa, Artificial Sequence
  Ascending Stalk Strand
GTDYTID

SEQ ID NO: 109 - protein, 6 aa, Artificial Sequence
  Ascending Stalk Strand
GTDYTI

SEQ ID NO: 110 - protein, 11 aa, Artificial Sequence
  Ascending Stalk Strand
DKGDSDYDYNL

SEQ ID NO: 111 - protein, 10 aa, Artificial Sequence
  Ascending Stalk Strand
DKGDSDYDYN

SEQ ID NO: 112 - protein, 9 aa, Artificial Sequence
  Ascending Stalk Strand
DKGDSDYDY

SEQ ID NO: 113 - protein, 8 aa, Artificial Sequence
  Ascending Stalk Strand
DKGDSDYD

SEQ ID NO: 114 - protein, 7 aa, Artificial Sequence
  Ascending Stalk Strand
DKGDSDY

SEQ ID NO: 115 - protein, 6 aa, Artificial Sequence
  Ascending Stalk Strand
DKGDSD

SEQ ID NO: 116 - protein, 17 aa, Artificial Sequence
  Ascending Stalk Strand
YGPNYEEWGDYLATLDV

SEQ ID NO: 117 - protein, 16 aa, Artificial Sequence
  Ascending Stalk Strand
GPNYEEWGDYLATLDV

SEQ ID NO: 118 - protein, 15 aa, Artificial Sequence
  Ascending Stalk Strand
PNYEEWGDYLATLDV

SEQ ID NO: 119 - protein, 14 aa, Artificial Sequence
  Ascending Stalk Strand
NYEEWGDYLATLDV

SEQ ID NO: 120 - protein, 13 aa, Artificial Sequence
  Ascending Stalk Strand
YEEWGDYLATLDV

SEQ ID NO: 121 - protein, 12 aa, Artificial Sequence
  Ascending Stalk Strand
EEWGDYLATLDV

SEQ ID NO: 122 - protein, 15 aa, Artificial Sequence
  Descending Stalk Strand
YDFYDGYYNYHYMDV

SEQ ID NO: 123 - protein, 14 aa, Artificial Sequence
  Descending Stalk Strand
DFYDGYYNYHYMDV

SEQ ID NO: 124 - protein, 13 aa, Artificial Sequence
  Descending Stalk Strand
FYDGYYNYHYMDV

SEQ ID NO: 125 - protein, 12 aa, Artificial Sequence
  Descending Stalk Strand
YDGYYNYHYMDV

SEQ ID NO: 126 - protein, 11 aa, Artificial Sequence
  Descending Stalk Strand
DGYYNYHYMDV

SEQ ID NO: 127 - protein, 10 aa, Artificial Sequence
  Descending Stalk Strand
GYYNYHYMDV

SEQ ID NO: 128 - protein, 9 aa, Artificial Sequence
  Descending Stalk Strand
YYNYHYMDV

SEQ ID NO: 129 - protein, 15 aa, Artificial Sequence
  Descending Stalk Strand
YDFNDGYYNYHYMDV

SEQ ID NO: 130 - protein, 14 aa, Artificial Sequence
  Descending Stalk Strand
DFYDGYYNYHYMDV

SEQ ID NO: 131 - protein, 13 aa, Artificial Sequence
  Descending Stalk Strand
FYDGYYNYHYMDV

SEQ ID NO: 132 - protein, 12 aa, Artificial Sequence
  Descending Stalk Strand
YDGYYNYHYMDV

SEQ ID NO: 133 - protein, 11 aa, Artificial Sequence
  Descending Stalk Strand
DGYYNYHYMDV

SEQ ID NO: 134 - protein, 10 aa, Artificial Sequence
  Descending Stalk Strand
GYYNYHYMDV

SEQ ID NO: 135 - protein, 16 aa, Artificial Sequence
  Descending Stalk Strand
QGIRYQGSGTFWYFDV

SEQ ID NO: 136 - protein, 15 aa, Artificial Sequence
  Descending Stalk Strand
GIRYQGSGTFWYFDV

SEQ ID NO: 137 - protein, 14 aa, Artificial Sequence
  Descending Stalk Strand
IRYQGSGTFWYFDV

SEQ ID NO: 138 - protein, 13 aa, Artificial Sequence
  Descending Stalk Strand
RYQGSGTFWYFDV

SEQ ID NO: 139 - protein, 12 aa, Artificial Sequence
  Descending Stalk Strand
YQGSGTFWYFDV

SEQ ID NO: 140 - protein, 11 aa, Artificial Sequence
  Descending Stalk Strand
QGSGTFWYFDV

SEQ ID NO: 141 - protein, 10 aa, Artificial Sequence
  Descending Stalk Strand
GSGTFWYFDV

SEQ ID NO: 142 - protein, 9 aa, Artificial Sequence
  Descending Stalk Strand
SGTFWYFDV

SEQ ID NO: 143 - protein, 8 aa, Artificial Sequence
  Descending Stalk Strand
GTFWYFDV

SEQ ID NO: 144 - protein, 13 aa, Artificial Sequence
  Descending Stalk Strand
YNLGYSYFYYMDG

SEQ ID NO: 145 - protein, 12 aa, Artificial Sequence
  Descending Stalk Strand
NLGYSYFYYMDG

SEQ ID NO: 146 - protein, 11 aa, Artificial Sequence
  Descending Stalk Strand
LGYSYFYYMDG

SEQ ID NO: 147 - protein, 10 aa, Artificial Sequence
  Descending Stalk Strand
GYSYFYYMDG

SEQ ID NO: 148 - protein, 9 aa, Artificial Sequence
  Descending Stalk Strand
YSYFYYMDG

SEQ ID NO: 149 - protein, 8 aa, Artificial Sequence
  Descending Stalk Strand
SYFYYMDG

SEQ ID NO: 150 - protein, 2 aa, Artificial Sequence
  Linker
GS

SEQ ID NO: 151 - protein, 3 aa, Artificial Sequence
  Linker
GGS

SEQ ID NO: 152 - protein, 6 aa, Artificial Sequence
  Linker
GGSGGS

SEQ ID NO: 153 - protein, 9 aa, Artificial Sequence
  Linker
GGSGGSGGS

SEQ ID NO: 154 - protein, 5 aa, Artificial Sequence
  Linker
GGGGS

SEQ ID NO: 155 - DNA, 662 bp, Artificial Sequence
  human light chain lambda region
tcacgaattcgcaggccgtcctgaaccagccaagcagcgtctccgggtctctggggcagcgggtctcaatcacctgtagcgggtcttcctccaatgtcggcaacggctacgtgtcttggtatcagctgatccctggcagtgccccacgaaccctgatctacggcgacacatccagagcttctggggtccccgatcggttctcagggagcagatccggaaacacagctactctgaccatcagctccctgcaggctgaggacgaagcagattatttctgcgcatctgccgaggactctagttcaaatgccgtgtttggaagcggcaccacactgacagtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataa

SEQ ID NO: 156 - protein, 216 aa, Artificial Sequence
  human VL1-51
QSVLTQPPSVSAAPGQKVTISCSGSSSNIGNNYVSWYQQLPGTAPKLLIYDNNKRPSGIPDRFSGSKSGTSATLGITGLQTGDEADYYCASAEDSSSNAVFGSGTTLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 157 - DNA, 39 bp, Artificial Sequence
  BLV1H12 ascending stalk region
tgtacttctgtgcaccaggaaacaaaaaaataccaaacc

SEQ ID NO: 158 - protein, 12 aa, Artificial Sequence
  BLV1H12 ascending stalk region
TSVHQETKKYQT

SEQ ID NO: 159 - protein, 13 aa, Artificial Sequence
  BLV1H12 ascending stalk region
CTSVHQETKKYQT

SEQ ID NO: 160 - DNA, 39 bp, Artificial Sequence
  Descending stalk region
tcctatacttacaattacgaatggcacgtcgatgtctgg

SEQ ID NO: 161 - protein, 13 aa, Artificial Sequence
  Descending stalk region
SYTYNYEWHVDVW

SEQ ID NO: 162 - DNA, 117 bp, Artificial Sequence
  BLV1H12 knob sequence
tgtccggaggattatacttataatccacgttgccctcagcagtatggttggagtgactgtgattgtatgggcgataggtttgggggttactgtcgacaggatggttgtagtaattat

SEQ ID NO: 163 - DNA, 9 bp, Artificial Sequence
  Coding sequencing for N-terminal GGS linker
ggtggatca

SEQ ID NO: 164 - DNA, 9 bp, Artificial Sequence
  Coding sequence for C-terminal GSG linker
ggttcagga

SEQ ID NO: 165 - protein, 133 aa, Artificial Sequence [sequence omitted]

SEQ ID NO: 166 - DNA, 399 bp, Artificial Sequence
  IL2 coding sequence
gcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgact

SEQ ID NO: 167 - DNA, 513 bp, Artificial Sequence
  Chimeric ultralong bovine heavy chain sequence
caggtgcagctgcgggagtcgggccccagcctgatgaagccgtcacagaccctctccctcacctgcacggtctctggatcttcattgaacgacaagtctgtaggctgggtccgccaggctccagggaaggcgctgcagtggctcggtagtgtggacactagtggaaacacagactataacccaggcctgaaatcccggctcagcatcaccaaggacaactccaagagccgaatctctcttacagtgactggcatgacaactgaagactcggccacatactactgtacttctgtgcaccaggaaacaaaaaaataccaaagttgtccggaggattatacttataatccacgttgccctcagcagtatggttggagtgactgtgattgtatgggcgataggtttgggggttactgtcgacaggatggttgtagtaattatagttatacttacaattacgaatggcacgtcgatgtctggggccaaggactcctggtcaccgtctcctcagctagc

SEQ ID NO: 168 - DNA, 651 bp, Artificial Sequence
  BLV1H12 Light Chain-
caggccgtcctgaaccagccaagcagcgtctccgggtctctggggcagcgggtctcaatcacctgtagcgggtcttcctccaatgtcggcaacggctacgtgtcttggtatcagctgatccctggcagtgccccacgaaccctgatctacggcgacacatccagagcttctggggtccccgatcggttctcagggagcagatccggaaacacagctactctgaccatcagctccctgcaggctgaggacgaagcagattatttctgcgcatctgccgaggactctagttcaaatgccgtgtttggaagcggcaccacactgacagtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcataa

SEQ ID NO: 169 - DNA, 311 bp, Artificial Sequence
  Human heavy chain variable region sequence 4-39
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagacacacagtgagggg

SEQ ID NO: 170 - DNA, 288 bp, Artificial Sequence
  Human heavy chain variable region sequence 4-59*03
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccaattctccctgaagctgagctctgtgaccgctgcggacacggccgtgtattactgtgcg

SEQ ID NO: 171 - DNA, 291 bp, Artificial Sequence
  Human heavy chain variable region sequence 4-34*09
caggtgcagctgcaggagtcgggcccaggactggtgaagccttcacagaccctgtccctcacctgcgctgtctatggtgggtccttcagtggttactactggagctggatccgccagcccccagggaagggactggagtggattggggaaatcaatcatagtggaagcaccaactacaacccgtccctcaagagtcgagttaccatatcagtagacacgtctaagaaccagttctccctgaagctgagctctgtgactgccgcggacacggccgtgtattactgtgcgaga

SEQ ID NO: 172 - DNA, 290 bp, Artificial Sequence
  Human heavy chain variable region sequence 4-34*02
caggtgcagctacaacagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgag

SEQ ID NO: 173 - protein, 98 aa, Artificial Sequence
  Human germline light chain variable region sequence VL1-47
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSG

SEQ ID NO: 174 - protein, 99 aa, Artificial Sequence
  Human germline light chain variable region sequence VL1-40*1
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSG

SEQ ID NO: 175 - protein, 98 aa, Artificial Sequence
  Human germline light chain variable region sequence VL1-51*01
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSA

SEQ ID NO: 176 - protein, 99 aa, Artificial Sequence
  Human germline light chain variable region sequence VL2-18*02
QSALTQPPSVSGSPGQSVTISCTGTSSDVGSYNRVSWYQQPPGTAPKLMIYEVSNRPSGVPDRFSGSKSGNTASLTISGLQAEDEADYYCSSYTSSSTF

SEQ ID NO: 177 - DNA, 296 bp, Artificial Sequence
  Human germline light chain variable region sequence VL1-47
cagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggtcc

SEQ ID NO: 178 - DNA, 299 bp, Artificial Sequence
  Human germline light chain variable region sequence VL1-40*1
cagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtggttc

SEQ ID NO: 179 - DNA, 296 bp, Artificial Sequence
  Human germline light chain variable region sequence VL1-51*01
cagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgctgg

SEQ ID NO: 180 - DNA, 297 bp, Artificial Sequence
  Human germline light chain variable region sequence VL2-18*02
cagtctgccctgactcagcctccctccgtgtccgggtctcctggacagtcagtcaccatctcctgcactggaaccagcagtgacgttggtagttataaccgtgtctcctggtaccagcagcccccaggcacagcccccaaactcatgatttatgaggtcagtaatcggccctcaggggtccctgatcgcttctctgggtccaagtctggcaacacggcctccctgaccatctctgggctccaggctgaggacgaggctgattattactgcagctcatatacaagcagcagcactttc

SEQ ID NO: 181 - protein, 216 aa, Artificial Sequence
  B15 Humanized Light Chain
QAVLNQPSSVSGSLGQKVTISCSGSSSNIGNNYVSWYQQLPGTAPKLLIYGDTKRPSGIPDRFSGSKSGTSATLGITGLQTGDEADYYCASAEDSSSNAVFGSGTTLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 182 - protein, 94 aa, Artificial Sequence
  B15 V1 Region
QVQLRESGPSLVKPSQTLSLTCTASGFSLSDKAVGWVRQAPGKALEWLGSIDTGGNTGYNPGLKSRLSITKDNSKSQVSLSVSSVTTEDSATYY

SEQ ID NO: 183 - protein, 13 aa, Artificial Sequence
  Ascending Stalk Region
CSTVHQETKKYQT

SEQ ID NO: 184 - protein, 13 aa, Artificial Sequence
  Ascending Stalk Region
CATVHQETKKYQT

SEQ ID NO: 185 - protein, 13 aa, Artificial Sequence
  Ascending Stalk Region
CTTVYQETKKYQT

SEQ ID NO: 186 - protein, 3 aa, Artificial Sequence
  Linker
GSG

SEQ ID NO: 187 - protein, 330 aa, Artificial Sequence
  Human IgG1 heavy constant region with LALA mutation and K97R
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 330 aa, Artificial Sequence
  Human IgG1 heavy constant region with LALA mutation
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 580 aa, Artificial Sequence
  B15 Variant 1 Heavy Chain
QVQLRESGPSLVKPSQTLSLTCTASGFSLSDKAVGWVRQAPGKALEWLGSIDTGGNTGYNPGLKSRLSITKDNSKSQVSLSVSSVTTEDSATYYCSTVHQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDVWGQGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 580 aa, Artificial Sequence
  B15 Variant 2 Heavy Chain
QVQLRESGPSLVKPSQTLSLTCTASGFSLSDKAVGWVRQAPGKALEWLGSIDTGGNTGYNPGLKSRLSITKDNSKSQVSLSVSSVTTEDSATYYCATVHQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDVWGQGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 580 aa, Artificial Sequence
  B15 Variant 3 Heavy Chain
QVQLRESGPSLVKPSQTLSLTCTASGFSLSDKAVGWVRQAPGKALEWLGSIDTGGNTGYNPGLKSRLSITKDNSKSQVSLSVSSVTTEDSATYYCTTVYQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDVWGQGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 580 aa, Artificial Sequence
  B15 Variant 4 Heavy Chain
QVQLREWGAGLLKPSETLSLTCAVYGGSFSDKYWSWIRQPPGKGLEWIGSINHSGSTNYNPSLKSRVTISVDTSKNQFSLKLSSVTAADTAVYYCSTVHQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDVWGQGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 580 aa, Artificial Sequence
  B15 Variant 5 Heavy Chain
QVQLREWGAGLLKPSETLSLTCAVYGGSFSDKYWSWIRQPPGKGLEWIGSINHSGSTNYNPSLKSRVTISVDTSKNQFSLKLSSVTAADTAVYYCATVHQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDVWGQGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 580 aa, Artificial Sequence
  B15 Variant 6 Heavy Chain
QVQLREWGAGLLKPSETLSLTCAVYGGSFSDKYWSWIRQPPGKGLEWIGSINHSGSTNYNPSLKSRVTISVDTSKNQFSLKLSSVTAADTAVYYCTTVYQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDVWGQGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 39 aa, Artificial Sequence
  BLV1H12 Knob Sequence
CPDGYRERSDCSNRPACGTSDCCRVSVFGNCLTTLPVSY

SEQ ID NO: 196 - protein, 330 aa, Artificial Sequence [sequence omitted]

SEQ ID NO: 197 - protein, 94 aa, Artificial Sequence
  B15 Humanized V1 Region
QVQLREWGAGLLKPSETLSLTCAVYGGSFSDKYWSWIRQPPGKGLEWIGSINHSGSTNYNPSLKSRVTISVDTSKNQFSLKLSSVTAADTAVYY

SEQ ID NO: 198 - protein, 580 aa, Artificial Sequence
  B15 Variant 7 Heavy Chain
QVQLRESGPSLVKPSQTLSLTCTASGFSLSDKAVGWVRQAPGKALEWLGSIDTGGNTGYNPGLKSRLSITKDNSKSQVSLSVSSVTTEDSATYYCTSVHQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDVWGQGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 580 aa, Artificial Sequence
  B15 Variant 8 Heavy Chain
QVQLREWGAGLLKPSETLSLTCAVYGGSFSDKYWSWIRQPPGKGLEWIGSINHSGSTNYNPSLKSRVTISVDTSKNQFSLKLSSVTAADTAVYYCTSVHQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDVWGQGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 200 - protein, 250 aa, Artificial Sequence
  B15 Variant 1 Variable Heavy Region
QVQLRESGPSLVKPSQTLSLTCTASGFSLSDKAVGWVRQAPGKALEWLGSIDTGGNTGYNPGLKSRLSITKDNSKSQVSLSVSSVTTEDSATYYCSTVHQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDVWGQGLLVTVSS

SEQ ID NO: 201 - protein, 250 aa, Artificial Sequence
  B15 Variant 2 Variable Heavy Region
QVQLRESGPSLVKPSQTLSLTCTASGFSLSDKAVGWVRQAPGKALEWLGSIDTGGNTGYNPGLKSRLSITKDNSKSQVSLSVSSVTTEDSATYYCATVHQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDVWGQGLLVTVSS

SEQ ID NO: 202 - protein, 250 aa, Artificial Sequence
  B15 Variant 3 Variable Heavy Region
QVQLRESGPSLVKPSQTLSLTCTASGFSLSDKAVGWVRQAPGKALEWLGSIDTGGNTGYNPGLKSRLSITKDNSKSQVSLSVSSVTTEDSATYYCTTVYQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDVWGQGLLVTVSS

SEQ ID NO: 203 - protein, 250 aa, Artificial Sequence
  B15 Variant 4 Variable Heavy Region
QVQLREWGAGLLKPSETLSLTCAVYGGSFSDKYWSWIRQPPGKGLEWIGSINHSGSTNYNPSLKSRVTISVDTSKNQFSLKLSSVTAADTAVYYCSTVHQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDVWGQGLLVTVSS

SEQ ID NO: 204 - protein, 250 aa, Artificial Sequence
  B15 Variant 5 Variable Heavy Region
QVQLREWGAGLLKPSETLSLTCAVYGGSFSDKYWSWIRQPPGKGLEWIGSINHSGSTNYNPSLKSRVTISVDTSKNQFSLKLSSVTAADTAVYYCATVHQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDVWGQGLLVTVSS

SEQ ID NO: 205 - protein, 250 aa, Artificial Sequence
  B15 Variant 6 Variable Heavy Region
QVQLREWGAGLLKPSETLSLTCAVYGGSFSDKYWSWIRQPPGKGLEWIGSINHSGSTNYNPSLKSRVTISVDTSKNQFSLKLSSVTAADTAVYYCTTVYQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDVWGQGLLVTVSS

SEQ ID NO: 206 - protein, 145 aa, Artificial Sequence
  B15 Variant 1 and 4 Ultralong CDR3
CSTVHQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDV

SEQ ID NO: 207 - protein, 145 aa, Artificial Sequence
  B15 Variant 2 and 5 Ultralong CDR3
CATVHQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDV

SEQ ID NO: 208 - protein, 145 aa, Artificial Sequence
  B15 Variant 3 and 6 Ultralong CDR3
CTTVYQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDV

SEQ ID NO: 209 - protein, 145 aa, Artificial Sequence
  B15 Variant 7 and 8 Ultralong CDR3
CTSVHQETKKYQTGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGSYTYNYEWHVDV

SEQ ID NO: 210 - protein, 469 aa, Artificial Sequence
  Control Antibody Heavy Chain (No Knob)
QVQLRESGPSLVKPSQTLSLTCTASGFSLSDKAVGWVRQAPGKALEWLGSIDTGGNTGYNPGLKSRLSITKDNSKSQVSLSVSSVTTEDSATYYCTTVYQETKKYQSGGSGGSGGSSYTYNYEWHVDVWGQGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 469 aa, Artificial Sequence
  Humanized Control Antibody Heavy Chain (No Knob)
QVQLREWGAGLLKPSETLSLTCAVYGGSFSDKYWSWIRQPPGKGLEWIGSINHSGSTNYNPSLKSRVTISVDTSKNQFSLKLSSVTAADTAVYYCTTVYQETKKYQSGGSGGSGGSSYTYNYEWHVDVWGQGLLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0197525 A1
(43) Pub. Date: Jun. 19, 2025

(54) HUMANIZED CHIMERIC BOVINE ANTIBODIES AND METHODS OF USE

(71) Applicant: Minotaur Therapeutics, Inc., San Diego, CA (US)

(72) Inventors: Ruiqi HUANG, San Diego, CA (US); Vaughn SMIDER, San Diego, CA (US); Duncan MCGREGOR, San Diego, CA (US)

(73) Assignee: Minotaur Therapeutics, Inc., San Diego, CA (US)

(21) Appl. No.: 18/557,470

(22) PCT Filed: Apr. 27, 2022
(86) PCT No.: PCT/US2022/026602

§ 371 Date: Oct. 26, 2023

Related U.S. Application Data
(60) Provisional application No. 63/181,223, filed on Apr. 28, 2021.

(51) Int. Cl. C07K 16/46; A61K 39/00; A61K 47/68; A61P 37/04; C07K 14/54; C07K 14/715; C12N 5/0783

(52) U.S. Cl. CPC C07K 16/461; A61K 47/6813; A61P 37/04; C07K 14/5434; C07K 14/5443; C07K 14/7155; C12N 5/0646; A61K 2039/505; C07K 2317/24; C07K 2317/52; C07K 2317/565; C07K 2317/71; C07K 2317/732

(57) ABSTRACT

Provided are chimeric antibodies containing an ultralong CDR3, such as based on a bovine antibody sequence or a humanized sequence thereof, in which a portion of the CDR3 of the heavy chain is replaced by a heterologous sequence, for instance that of interleukin (IL)-15 or IL-2, and related antibodies. Among the molecules of the present disclosure are chimeric IL-15 modified antibody molecules that are further linked or complexed with an extracellular portion of IL15Rα, such as the IL15Rα sushi domain. The present disclosure also provides methods of making and using the chimeric antibodies.

### CROSS-REFERENCE TO RELATED APPLICATIONS

This application claims priority to U.S. Provisional Application No. 63/181,223, filed Apr. 28, 2021, the contents of which are hereby incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 166262000340SeqList.txt, created Apr. 23, 2022, which is 152 kilobytes in size. The information in the electronic format of the Sequence Listing is incorporated by reference in its entirety.

### FIELD

The present disclosure relates to chimeric antibodies containing an ultralong CDR3, such as based on a bovine antibody sequence or a humanized sequence thereof, in which a portion of the CDR3 of the heavy chain is replaced by a heterologous sequence, for instance that of interleukin (IL)-15 or IL-2, and related antibodies. Among the molecules of the present disclosure are chimeric IL-15 modified antibody molecules that are further linked or complexed with an extracellular portion of IL15Rα, such as the IL15Rα sushi domain. The present disclosure also provides methods of making and using the chimeric antibodies.

### BACKGROUND

Antibodies are natural proteins that the vertebrate immune system forms in response to foreign substances (antigens), primarily for defense against infection. Antibodies contain complementarity determining regions (CDRs) that mediate binding to a target antigen. Some bovine antibodies have unusually long variable heavy (VH) CDR3 sequences compared to other vertebrates. These long CDR3s, which can be up to 70 amino acids long, can form unique domains that protrude from the antibody surface, thereby permitting a unique antibody platform.

Interleukin (IL)-15 and IL-2 are cytokines that stimulate the proliferation and cytotoxicity of cytotoxic T lymphocytes and natural killer (NK) cells, and thus are immunotherapeutic candidates for cancer treatment. However, such cytokines can be difficult to express as a stable soluble protein and often have a short half-life in vitro and in vivo. There remains a need for improved cytokine therapeutics, such as IL-2 or IL-15 therapeutics, particularly for use in treating cancer.

### SUMMARY

Provided herein in some embodiments is a chimeric modified antibody, comprising a heavy chain comprising: (a) a modified variable heavy (VH) region of a bovine antibody or antigen-binding fragment or a humanized sequence thereof, wherein the modified VH region comprises a modified ultralong CDR3 wherein at least a portion of an ultralong CDR3 of the bovine antibody or antigen-binding fragment or a humanized sequence thereof is replaced by a cytokine sequence or a biologically active portion thereof; and (b) a modified human IgG heavy chain constant region with reduced effector activity compared to a wild-type human IgG heavy chain constant region.

In some of any embodiments, the cytokine sequence or biologically active portion thereof replaces a knob region of the ultralong CDR3 region of the bovine antibody or antigen-binding fragment or the humanized sequence thereof.

In some of any embodiments, the cytokine sequence or biologically active portion thereof is between an ascending stalk strand and a descending stalk strand of the modified ultralong CDR3, wherein the ascending stalk strand of the modified ultralong CDR3 is a variant compared to an ascending stalk strand of the ultralong CDR3 of the bovine antibody or antigen-binding fragment or a humanized sequence thereof. In some of any embodiments, the cytokine sequence or biologically active portion thereof is linked to the ascending stalk strand and/or to the descending stalk strand of the modified ultralong CDR3 via a flexible linker, optionally a GGS or GSG linker. In some of any embodiments, the cytokine sequence or biologically active portion thereof is linked to the ascending stalk strand and to the descending stalk strand of the modified ultralong CDR3 via a flexible linker. In some of any embodiments, the flexible linker is a GGS linker. In some of any embodiments, the linker is a GSG linker. In some of any embodiments, the cytokine sequence or biologically active portion thereof is linked to the ascending stalk strand of the modified ultralong CDR3 via a GGS linker and to the descending stalk strand of the modified ultralong CDR3 via a GSG linker.

In some of any embodiments, the ascending stalk strand comprises the sequence CX₂TVX₅QETKKYQT, wherein X₂ and X₅ are any amino acid.

Provided herein in some embodiments is a chimeric modified antibody, comprising a heavy chain comprising a modified variable heavy (VH) region of a bovine antibody or antigen-binding fragment or a humanized sequence thereof, wherein the modified VH region comprises a modified ultralong CDR3 in which at least a portion of an ultralong CDR3 region of the bovine antibody or antigen-binding fragment or a humanized sequence thereof is replaced by a heterologous sequence, wherein the heterologous sequence is between an ascending stalk strand and a descending stalk strand of the modified ultralong CDR3, wherein the ascending stalk strand of the modified ultralong CDR3 comprises the sequence CX₂TVX₅QETKKYQT, wherein X₂ and X₅ are any amino acid.

In some of any embodiments, X₂ is Ser, Thr, Gly, Asn, Ala, or Pro, and X₅ is His, Gln, Arg, Lys, Gly, Thr, Tyr, Phe, Trp, Met, Ile, Val, or Leu. In some of any embodiments, X₂ is Ser, Ala, or Thr, and X₅ is His or Tyr.

In some of any embodiments, the ascending stalk strand of the modified ultralong CDR3 comprises the sequence set forth in any of SEQ ID NOs: 183-185. In some of any embodiments, the sequence of the ascending stalk strand of the modified ultralong CDR3 is set forth in any of SEQ ID NOs: 183-185.

In some of any embodiments, the ascending stalk strand of the modified ultralong CDR3 comprises the sequence set forth in SEQ ID NO: 185. In some of any embodiments, the sequence of the ascending stalk strand of the modified ultralong CDR3 is set forth in SEQ ID NO: 185.

In some of any embodiments, the heterologous sequence replaces a knob region of the ultralong CDR3 region of the bovine antibody or antigen-binding fragment or the humanized sequence thereof. In some of any embodiments, the heterologous sequence is linked to the ascending stalk strand and/or to the descending stalk strand of the modified ultralong CDR3 via a flexible linker, optionally a GGS or GSG linker. In some of any embodiments, the flexible linker is a GGS linker. In some of any embodiments, the linker is a GSG linker. In some of any embodiments, the heterologous sequence is linked to the ascending stalk strand and to the descending stalk strand of the modified ultralong CDR3 via a flexible linker. In some of any embodiments, the flexible linker is a GGS linker. In some of any embodiments, the linker is a GSG linker. In some of any embodiments, the heterologous sequence is linked to the ascending stalk strand of the modified ultralong CDR3 via a GGS linker and to the descending stalk strand of the modified ultralong CDR3 via a GSG linker.

In some of any embodiments, the heterologous sequence comprises a cytokine sequence or a biologically active portion thereof.

In some of any embodiments, the heavy chain further comprises a human IgG heavy chain constant region. In some of any embodiments, the human IgG heavy chain constant region is a modified human IgG heavy chain constant region with reduced effector activity compared to a wild-type human IgG heavy chain constant region.

In some of any embodiments, the human IgG is human IgG1.

In some of any embodiments, the modified human IgG heavy chain constant region is modified to reduce FcR binding. In some of any embodiments, the reduced effector activity comprises reduced antibody-dependent cell-mediated cytotoxicity (ADCC).

In some of any embodiments, the modified human IgG heavy chain constant region is altered at one or more of positions Glu233 (E233), Leu 234 (L234), Leu235 (L235), Asp265 (D265), Asp270 (D270), Asn297 (N297), Ser298 (S298), Asn325 (N325), Ala327 (A327), and Pro329 (P329). In some of any embodiments, the modified human IgG heavy chain constant region comprises one or more mutations selected from Leu234Ala (L234A), Leu235Ala (L235A), Leu235Glu (L235E), Asp265Asn (D265N), Asp265Ala (D265A), Asp270Asn (D270N), Ser298Asn (S298N), Asn325Glu (N325E), Ala327Ser (A327S), Pro329Ala (P329A), and Pro239Gly (P329G).

In some of any embodiments, the modified human IgG heavy chain constant region is altered at two or more of positions Glu233 (E233), Leu 234 (L234), Leu235 (L235), Asp265 (D265), Asp270 (D270), Asn297 (N297), Ser298 (S298), Asn325 (N325), Ala327 (A327), and Pro329 (P329). In some of any embodiments, the modified human IgG heavy chain constant region comprises Leu234Ala and Leu235Ala (L234A/L235A) mutations; Leu234Val and Leu235Ala (L234V/L235A) mutations; Leu234Ala, Leu235Ala, and Asn297Ala (L234A/L235A/N297A) mutations; Leu234Ala, Leu235Ala, and Pro239Ala (L234A/L235A/P329A) mutations; Asp265Ala and Pro329Ala (D265A/P329A) mutations; Asp265Ala and Pro329Gly (D265A/P329G) mutations; Leu234Ala, Leu235Ala, and Asp265Ala (L234A/L235A/D265A) mutations; Leu234Ala, Leu235Ala, and Pro329Gly (L234A/L235A/P329G) mutations; or Leu234Ala, Leu235Ala, Asp265Ala, and Pro329Gly (L234A/L235A/D265A/P329G) mutations.

In some of any embodiments, the modified human IgG heavy chain constant region comprises Leu234Ala and Leu235Ala (L234A/L235A) mutations.

In some of any embodiments, the modified human IgG heavy chain constant region comprises the sequence set forth in SEQ ID NO: 187 or SEQ ID NO: 188. In some of any embodiments, the modified human IgG heavy chain constant region comprises the sequence set forth in SEQ ID NO: 187. In some of any embodiments, the modified human IgG heavy chain constant region comprises the sequence set forth in SEQ ID NO: 188.

In some of any embodiments, the cytokine sequence or biologically active portion thereof comprises an interleukin-15 (IL-15) cytokine sequence or a biologically active portion thereof. In some of any embodiments, the cytokine sequence or biologically active portion thereof comprises a sequence of amino acids that exhibits at least at or about 85%, at least at or about 90%, at least at or about 92%, at least at or about 95%, at least at or about 96%, at least at or about 97%, at least at or about 98%, or at least at or about 99% sequence identity to SEQ ID NO: 1. In some of any embodiments, the cytokine sequence or biologically active portion thereof comprises the sequence set forth in SEQ ID NO: 1.

In some of any embodiments, the cytokine sequence or biologically active portion thereof comprises an interleukin-12 (IL-2) cytokine sequence or a biologically active portion thereof. In some of any embodiments, the cytokine sequence or biologically active portion thereof comprises a sequence of amino acids that exhibits at least at or about 85%, at least at or about 90%, at least at or about 92%, at least at or about 95%, at least at or about 96%, at least at or about 97%, at least at or about 98%, or at least at or about 99% sequence identity to SEQ ID NO: 165. In some of any embodiments, the cytokine sequence or biologically active portion thereof comprises the sequence of amino acids set forth in SEQ ID NO: 165.

In some of any embodiments, the bovine antibody or antigen-binding fragment is the bovine antibody BLV1H12 or an antigen-binding fragment thereof.

In some of any embodiments, the descending stalk strand comprises the sequence set forth in SEQ ID NO: 10.

In some of any embodiments, the ascending stalk strand comprises the sequence set forth in SEQ ID NO: 183, the cytokine sequence or biologically active portion thereof comprises the sequence of amino acids set forth in SEQ ID NO: 1, and the descending stalk strand comprises the sequence set forth in SEQ ID NO: 10; the ascending stalk strand comprises the sequence set forth in SEQ ID NO: 184, the cytokine sequence or biologically active portion thereof comprises the sequence of amino acids set forth in SEQ ID NO: 1, and the descending stalk strand comprises the sequence set forth in SEQ ID NO: 10; or the ascending stalk strand comprises the sequence set forth in SEQ ID NO: 185, the cytokine sequence or biologically active portion thereof comprises the sequence of amino acids set forth in SEQ ID NO: 1, and the descending stalk strand comprises the sequence set forth in SEQ ID NO: 10.

In some of any embodiments, the ascending stalk strand comprises the sequence set forth in SEQ ID NO: 183, the cytokine sequence or biologically active portion thereof comprises the sequence of amino acids set forth in SEQ ID NO: 1, and the descending stalk strand comprises the sequence set forth in SEQ ID NO: 10.

In some of any embodiments, the ascending stalk strand comprises the sequence set forth in SEQ ID NO: 184, the cytokine sequence or biologically active portion thereof comprises the sequence of amino acids set forth in SEQ ID NO: 1, and the descending stalk strand comprises the sequence set forth in SEQ ID NO: 10.

In some of any embodiments, the ascending stalk strand comprises the sequence set forth in SEQ ID NO: 185, the cytokine sequence or biologically active portion thereof comprises the sequence of amino acids set forth in SEQ ID NO: 1, and the descending stalk strand comprises the sequence set forth in SEQ ID NO: 10.

In some of any embodiments, the modified ultralong CDR3 comprises the sequence set forth in any of SEQ ID NOs: 206-208. In some of any embodiments, the modified ultralong CDR3 comprises the sequence set forth in SEQ ID NO: 208.

In some of any embodiments, the modified VH region is a variant of the VH region of BLV1H12.

In some of any embodiments, the heavy chain comprises the formula V1-X-V2-C, wherein the V1 region of the heavy chain comprises the sequence set forth in SEQ ID NO: 182; the X region comprises the modified ultralong CDR3; the V2 region comprises the sequence set forth in SEQ ID NO: 11; and the C region comprises a human IgG heavy chain constant region. In some embodiments, the human IgG heavy chain constant region is any as described herein. In some of any embodiments, the human IgG heavy chain constant region is a modified human IgG heavy chain constant region with reduced effector activity compared to a wild-type human IgG heavy chain constant region. In some embodiments, the modified human IgG heavy chain constant region is any as described herein.

In some of any embodiments, the heavy chain comprises the formula V1-X-V2-C, wherein the V1 region of the heavy chain comprises the sequence set forth in SEQ ID NO: 182; the X region comprises the modified ultralong CDR3; the V2 region comprises the sequence set forth in SEQ ID NO: 11; and the C region comprises the modified human IgG heavy chain constant region.

In some of any embodiments, the modified VH region comprises the sequence set forth in any of SEQ ID NOs: 200-202. In some of any embodiments, the modified VH region comprises the sequence set forth in SEQ ID NO: 202.

In some of any embodiments, the heavy chain comprises the sequence set forth in any of SEQ ID NOs: 189-191. In some of any embodiments, the heavy chain comprises the sequence set forth in SEQ ID NO: 191.

In some of any embodiments, the modified VH region is a variant of a humanized sequence of the VH region of BLV1H12.

In some of any embodiments, the heavy chain comprises the formula V1-X-V2-C, wherein the V1 region of the heavy chain comprises the sequence set forth in SEQ ID NO: 197 or a sequence that exhibits at least 65% sequence identity to SEQ ID NO: 197; the X region comprises the modified ultralong CDR3; the V2 region comprises the sequence set forth in SEQ ID NO: 11; and the C region comprises a human IgG heavy chain constant region. In some embodiments, the human IgG heavy chain constant region is any as described herein. In some of any embodiments, the human IgG heavy chain constant region is a modified human IgG heavy chain constant region with reduced effector activity compared to a wild-type human IgG heavy chain constant region. In some embodiments, the modified human IgG heavy chain constant region is any as described herein. In some embodiments, V1 region has a sequence identity that is at least 70%, at least 75%, at least 80%, at least 85%, at least 90%, or at least 95% to the sequence set forth in SEQ ID NO: 197.

In some of any embodiments, the heavy chain comprises the formula V1-X-V2-C, wherein the V1 region of the heavy chain comprises the sequence set forth in SEQ ID NO: 197 or a sequence that exhibits at least 65% sequence identity to SEQ ID NO: 197; the X region comprises the modified ultralong CDR3; the V2 region comprises the sequence set forth in SEQ ID NO: 11; and the C region comprises the modified human IgG heavy chain constant region. In some embodiments, V1 region has a sequence identity that is at least 70%, at least 75%, at least 80%, at least 85%, at least 90%, or at least 95% to the sequence set forth in SEQ ID NO: 197.

In some of any embodiments, the heavy chain comprises the formula V1-X-V2-C, wherein the V1 region of the heavy chain comprises the sequence set forth in SEQ ID NO: 197; the X region comprises the modified ultralong CDR3; the V2 region comprises the sequence set forth in SEQ ID NO: 11; and the C region comprises a human IgG heavy chain constant region. In some embodiments, the human IgG heavy chain constant region is any as described herein. In some of any embodiments, the human IgG heavy chain constant region is a modified human IgG heavy chain constant region with reduced effector activity compared to a wild-type human IgG heavy chain constant region. In some embodiments, the modified human IgG heavy chain constant region is any as described herein.

In some of any embodiments, the heavy chain comprises the formula V1-X-V2-C, wherein the V1 region of the heavy chain comprises the sequence set forth in SEQ ID NO: 197; the X region comprises the modified ultralong CDR3; the V2 region comprises the sequence set forth in SEQ ID NO: 11; and the C region comprises the modified human IgG heavy chain constant region.

In some of any embodiments, the modified VH region comprises the sequence set forth in any of SEQ ID NOs: 203-205. In some of any embodiments, the modified VH region comprises the sequence set forth in SEQ ID NO: 205.

In some of any embodiments, the heavy chain comprises the sequence set forth in any of SEQ ID NOs: 192-194. In some of any embodiments, the heavy chain comprises the sequence set forth in SEQ ID NO: 205.

In some of any embodiments, the chimeric modified antibody further comprises a light chain. In some of any embodiments, the chimeric modified antibody comprises a humanized light chain. In some of any embodiments, the humanized light chain comprises the sequence set forth in SEQ ID NO: 181 or a sequence that exhibits at least 85% sequence identity to SEQ ID NO: 181. In some of any embodiments, the humanized light chain comprises the sequence set forth in SEQ ID NO: 181. In some embodiments, the humanized light chain has a sequence identity that is at least at least 90%, at least 95%, or at least 95% to the sequence set forth in SEQ ID NO: 181.

In some of any embodiments, the antibody is a full length or intact antibody.

In some of any of the embodiments herein, the antibody is an antigen-binding fragment. In a further embodiment, the antigen-binding fragment is a Fab, Fab′-SH, Fv, scFv, or (Fab′)2 fragment. In some embodiments, the antibody is a Fab.

In some of any embodiments, the chimeric modified antibody is complexed with an extracellular domain of the IL15Rα comprising the IL15Rα sushi domain. In some of any embodiments, the extracellular domain of the IL15Rα comprising the IL15Rα sushi domain is non-covalently associated with the IL-15 sequence. In some of any embodiments, the extracellular domain of the IL15Rα comprising the IL15Rα sushi domain is linked to the light chain of the chimeric modified antibody, optionally linked via a peptide linker. In some of any embodiments, the extracellular domain of the IL15Rα comprising the IL15Rα sushi domain is linked via a peptide linker to the light chain of the chimeric modified antibody. In some of any embodiments, the extracellular domain of the IL15Rα comprising the IL15Rα sushi domain comprises the sequence set forth in SEQ ID NO: 2.

Provided herein in some embodiments is a polynucleotide encoding the chimeric modified antibody of any embodiments.

Provided herein in some embodiments polynucleotide encoding a heavy chain or a variable region thereof of the chimeric modified antibody of any embodiments.

Provided herein in some embodiments polynucleotide encoding a light chain or a variable region thereof of the chimeric modified antibody of any embodiments.

Provided herein in some embodiments is an expression vector comprising the polynucleotide of any embodiments.

Provided herein in some embodiments is a host cell comprising the polynucleotide or the expression vector of any embodiments.

In some of any embodiments, the host cell further comprises a polynucleotide or vector encoding an extracellular domain of the IL15Rα comprising the IL15Rα sushi domain. In some of any embodiments, the extracellular domain of the IL15Rα comprising the IL15Rα sushi domain comprises the sequence set forth in SEQ ID NO: 2.

Provided herein in some embodiments is a method of producing a chimeric modified antibody, comprising culturing the host cell of any embodiments under conditions for expression of the chimeric modified antibody by the host cell, optionally further comprising recovering or purifying the chimeric modified antibody. In some embodiments, the conditions are for expression of the chimeric modified antibody, the heavy chain or variable region thereof of the chimeric modified antibody, or the light chain or variable region thereof of the chimeric modified antibody by the host cell.

In some of any embodiments, the method further comprises recovering or purifying the chimeric modified antibody, the heavy chain or variable region thereof, or the light chain or variable region thereof.

In some of any embodiments, the method further comprises recovering or purifying the chimeric modified antibody.

Provided herein in some embodiments is a chimeric modified antibody produced by the method of any embodiments.

Provided herein in some embodiments is a chimeric modified antibody comprising the heavy chain or variable region thereof or the light chain or variable region thereof produced by the method of any embodiments.

Provided herein in some embodiments is a pharmaceutical composition comprising the chimeric modified antibody of any embodiments.

Provided herein in some embodiments is a method of stimulating immune cells, comprising contacting a population of immune cells with the chimeric modified antibody of any embodiments, thereby stimulating cells of the population of immune cells.

Provided herein in some embodiments is a method of expanding immune cells, comprising contacting a population of immune cells with the chimeric modified antibody of any embodiments, thereby promoting proliferation of cells of the population of immune cells.

In some of any embodiments, the population of immune cells comprises cells expressing an IL2/15Rβ and/or an IL2/15Rβ γc receptor subunit. In some of any embodiments, the population of immune cells comprises cells expressing an IL2/15Rβ and an IL2/15Rβ γc receptor subunit.

In some of any embodiments, the population of immune cells comprises T cells or natural killer (NK) cells. In some of any embodiments, the population of immune cells comprises T cells. In some of any embodiments, the population of immune cells comprises NK cells.

In some of any embodiments, the method is performed ex vivo or in vitro. In some of any embodiments, the method is performed in vivo upon administration of the chimeric modified antibody to a subject.

Provided herein in some embodiments is a method of treating a cancer in a subject, comprising administering to a subject a therapeutically effective amount of the chimeric modified antibody of any embodiments.

Provided herein in some embodiments is a method of treating a cancer in a subject, comprising administering to a subject the pharmaceutical composition of any embodiments.

In some of any embodiments, the method further comprises administering to the subject an anti-tumor agent. In some of any embodiments, the anti-tumor agent comprises a monoclonal antibody. In some of any embodiments, the anti-tumor agent comprises a checkpoint inhibitor. In some of any embodiments, the anti-tumor agent comprises a cell therapy, optionally a T cell therapy or an NK cell therapy. In some of any embodiments, the cell therapy is a T cell therapy. In some of any embodiments, the cell therapy is an NK cell therapy. In some of any embodiments, the cell therapy comprises cells expressing a chimeric antigen receptor (CAR).

In some of any embodiments, the anti-tumor agent is an agent for treating the cancer. In some of any embodiments, the anti-tumor agent is directed against an antigen associated with the cancer.

In some of any embodiments, the cell therapy comprises cells expressing an IL2/15Rβ and an IL2/15Rβ γc receptor subunit.

Provided herein in some embodiments is use of any of the provided chimeric modified antibodies in the manufacture of a medicament for treating a cancer in a subject.

Provided herein in some embodiments is use of a pharmaceutical composition comprising any of the provided chimeric modified antibodies in a method of treating a cancer in a subject.

In some of any embodiments, an anti-tumor agent is administered in combination with the pharmaceutical composition to the subject.

Provided herein in some embodiments is use of an anti-tumor agent and a pharmaceutical composition comprising any of the provided chimeric modified antibodies in a method of treating a cancer in a subject.

Provided herein in some embodiments is use of an anti-tumor agent in a method of treating a cancer in a subject, wherein the anti-tumor agent is administered in combination with a pharmaceutical composition comprising any of the provided chimeric modified antibodies.

In some of any embodiments, the method comprises administering the pharmaceutical composition to the subject.

In some of any embodiments, the method comprises administering the anti-tumor agent to the subject.

In some of any embodiments, the pharmaceutical composition and the anti-tumor agent are separately administered to the subject.

In some of any embodiments, the method is any as described herein.

Provided herein in some embodiments is use of a combination of any of the provided chimeric modified antibodies and an anti-tumor agent in the manufacture of a medicament for treating a cancer in a subject.

Provided herein in some embodiments is use of an anti-tumor agent in the manufacture of a medicament for treating a cancer in a subject, wherein the anti-tumor agent is administered in combination with a pharmaceutical composition comprising any of the provided chimeric modified antibodies.

In some of any embodiments, the anti-tumor agent is an agent for treating the cancer. In some of any embodiments, the anti-tumor agent is directed against an antigen associated with the cancer.

In some of any embodiments, the anti-tumor agent comprises a monoclonal antibody. In some of any embodiments, the anti-tumor agent comprises a checkpoint inhibitor. In some of any embodiments, the anti-tumor agent comprises a cell therapy. In some of any embodiments, the cell therapy comprises cells expressing an IL2/15Rβ and an IL2/15Rβ γc receptor subunit. In some of any embodiments, the cell therapy is a T cell therapy. In some of any embodiments, the cell therapy is an NK cell therapy. In some of any embodiments, the cell therapy comprises cells expressing a chimeric antigen receptor (CAR).

Provided herein in some embodiments is a pharmaceutical composition comprising any of the provided chimeric modified antibodies for use in a method of treating a cancer in a subject.

In some of any embodiments, an anti-tumor agent is administered in combination with the pharmaceutical composition to the subject.

Provided herein in some embodiments is a combination therapy comprising a pharmaceutical composition comprising of any of the provided chimeric modified antibodies and an anti-tumor agent for use in a method of treating a cancer in a subject.

Provided herein in some embodiments is an anti-tumor agent for use in a method of treating a cancer in a subject, wherein the anti-tumor agent is administered in combination with any of the provided chimeric modified antibodies.

In some of any embodiments, the method comprises administering the pharmaceutical composition to the subject.

In some of any embodiments, the method comprises administering the anti-tumor agent to the subject.

In some of any embodiments, the pharmaceutical composition and the anti-tumor agent are separately administered to the subject.

In some of any embodiments, the method is any as described herein.

In some of any embodiments, the anti-tumor agent is an agent for treating the cancer. In some of any embodiments, the anti-tumor agent is directed against an antigen associated with the cancer.

In some of any embodiments, the anti-tumor agent comprises a monoclonal antibody. In some of any embodiments, the anti-tumor agent comprises a checkpoint inhibitor. In some of any embodiments, the anti-tumor agent comprises a cell therapy. In some of any embodiments, the cell therapy comprises cells expressing an IL2/15Rβ and an IL2/15Rβ γc receptor subunit. In some of any embodiments, the cell therapy is a T cell therapy. In some of any embodiments, the cell therapy is an NK cell therapy. In some of any embodiments, the cell therapy comprises cells expressing a chimeric antigen receptor (CAR).

### BRIEF DESCRIPTION OF THE DRAWINGS

FIG. 1A and FIG. 1B depict schematic representations of the generated fusion antibody constructs. FIG. 1A shows the crystal structure of BLV1H12, depicting how the two-β-stranded stalk protrudes from the bovine VH immunoglobulin domain and terminates in an unusual three disulfide-linked knob domain (left), and the crystal structure of the BLV1H12-IL-15 Rαsushi (B15_Rαsushi) variant, in which the knob region has been replaced with an IL-15 cytokine sequence and the construct further contains an IL15Rα sushi domain (right). FIG. 1B depicts the different fusion antibody constructs BLV1H12-IL-15 (B15), BLV1H12-IL-15 Rαsushi (B15_Rαsushi), and BLV1H12-IL-15 GS-Rαsushi (B15_GS_Rαsushi).

FIG. 2 shows the activation of the IL2/15Rβ and γc receptor subunits and STAT5 signaling by chimeric BLV1H12-IL-15 (B15) fusion antibodies, through induction and secretion of the STAT5 inducible alkaline phosphatase (SEAP) reporter gene in HEK-Blue IL2 reporter cells.

FIG. 3A-3C show results of a rodent study in which rats were administered chimeric B15 fusion antibodies both with and without the IL15Rα sushi domain. FIG. 3A-3C show body weight (FIG. 3A), natural killer (NK) cell percentages (FIG. 3B), and CD8 T cell percentages (FIG. 3C) of rats following administration of the B15 fusion antibodies.

FIG. 4A-4D show results of a non-human primate study in which monkeys were administered humanized chimeric B15 fusion antibodies both with and without the IL15Rα sushi domain. FIG. 4A-4D show body weight (FIG. 4A), mature and cytotoxic T cell counts (FIG. 4B), NK and helper T cell counts (FIG. 4C), and B cell and monocyte counts (FIG. 4D) of monkeys before and after administration of the humanized B15 fusion antibodies.

### DETAILED DESCRIPTION

Provided herein are chimeric fusion antibodies in which a heterologous sequence, e.g., a cytokine sequence like an IL-15 or IL-2 sequence, or a biologically active portion thereof, replaces a portion of an ultralong CDR3 region of a heavy chain of a bovine (cow) antibody or a humanized sequence thereof. In some embodiments, the ultralong CDR3 region contains an ascending stalk region, a knob region, and a descending stalk region, such as present in bovine antibodies, in which all or a portion of the knob region is replaced by the cytokine sequence. In some embodiments, the cytokine sequence is that of IL-2 or a biologically active portion thereof, for example with the sequence set forth in SEQ ID NO:165. In some embodiments, the cytokine sequence is that of IL-15 or a biologically active portion thereof, for example with the sequence set forth in SEQ ID NO:1. In some embodiments, a further portion of the ultralong CDR3 region, e.g., the ascending stalk strand, is modified relative to that of the bovine antibody or humanized sequence thereof. In some embodiments, the heavy chain constant region of the provided chimeric antibodies is modified, e.g., mutated, in order to reduce effector activity of the provided chimeric antibodies, for instance reduced as compared to a wild-type heavy chain constant region. Also provided herein are variant chimeric IL-15 modified antibodies that include such antibodies linked or complexed with an extracellular portion of IL15Rα, such as the IL15Rα sushi domain (e.g., set forth in SEQ ID NO:2).

IL-15 and IL-2 are pleiotropic cytokines that play important roles in both innate and adaptive immunity. IL-15 was originally described, like IL-2, as a T cell growth factor. For example, IL-15 is involved in the generation of multiple lymphocyte subsets, including natural killer (NK) cells, NK-T cells, and memory CD8 T cells. IL-15 is also a chemotactic for T-cells, acts on neutrophils to induce morphological cell shape changes, and stimulates IL-8 production. Both cytokines belong to the four α-helix bundle family, and their membrane receptors share two subunits (the IL-2R/IL-15R β and γ chains) responsible for signal transduction. IL-15 functions through the trimeric IL-15R complex, which is made up of a high affinity binding α-chain (IL-15Rα) and the common IL-2R β- and γ-chains. The IL-2Rβ/γ complex is an intermediate affinity receptor for both cytokines that is expressed by most NK cells and can be activated in vitro by nanomolar concentrations of IL-2 or IL-15 (Wei et al. J Immunol. 2001, 167(1)277-282; Mortier et al. J Biol Chem. 2006, 281 (3): 1612-1619).

The IL-15Rα and IL-2Rα subunits form a sub-family of cytokine receptors containing an extracellular portion at their N terminus that is a so-called “sushi” structural domain (one in IL-15Rα and two in IL-2Rα), which is also found in complement or adhesion molecules. The IL-15Rα Sushi domain is a common motif in protein-protein interaction. Sushi domains are also known as short consensus repeats or type 1 glycoprotein motifs. They have been identified on a number of protein-binding molecules, including complement components C1r, C1s, factor H, and C2m as well as the nonimmunologic molecules factor XIII and β₂-glycoprotein. A typical Sushi domain has approximately 60 amino acid residues and contains four cysteines. The first cysteine forms a disulfide bond with the third cysteine, and the second cysteine forms a disulfide bridge with the fourth cysteine. The two disulfide bonds are essential to maintain the tertiary structure of the protein (Kato et al. Biochemistry. 1991, 30:11687; Bottenus et al. Biochemistry 1990, 29:11195; Ranganathan et al. Pac. Symp. Biocomput. 2000, 00:155). The high affinity receptor α (IL15Rα) is involved in increasing IL-15 mediated trans signaling to the receptor β and γ subunits (IL2/15Rβ and γc).

IL-2 can stimulate the proliferation, activation, and, in some cases, cytotoxicity of cytotoxic T lymphocytes and natural killer (NK) cells. IL-15 can stimulate the proliferation, activation, and, in some cases, cytotoxicity of cytotoxic T lymphocytes and natural killer (NK) cells. Although these activities make IL-2 and IL-15 desirable for therapeutic uses, IL-2 and IL-15 are difficult to express as a stable soluble protein and have a short half-life in vitro and in vivo.

The provided embodiments address these problems. Among the provided embodiments are chimeric antibodies in which an IL-2 or IL-15 cytokine sequence or a biologically active portion thereof replaces all or a portion of the knob region of a bovine antibody or a humanized variant thereof. The provided antibodies containing an IL-15 cytokine sequence or biologically active portion thereof can further be linked or complexed with an extracellular portion of IL15Rα, such as the IL15Rα sushi domain, to further mediate IL15 activity. It is found herein that the provided chimeric antibodies, including chimeric IL-15 antibodies (e.g., B15) and variants thereof complexed or linked with an extracellular portion of IL15Rα, can be expressed and purified similarly to typical human antibodies, and exhibit efficient binding and activity to IL2/15Rβ and γc subunits. In particular, the provided antibodies function similarly to soluble IL-15 in in vitro signaling assays, but can be easily produced in mammalian cells and with increased stability. In addition, it is found herein that the provided antibodies exhibit biological activity in vivo, including to induce proliferation of immune cells such as NK cells and T cells.

In addition, in some embodiments the provided antibodies have reduced effector activity, for instance have been modified or mutated to have reduced effector activity. In some embodiments, the provided antibodies are modified to reduce FcR binding and/or to reduce mediation or promotion of antibody-dependent cell-mediated cytotoxicity (ADCC). In some aspects, methods of using or use of the provided antibodies afford certain advantages, for instance the ability to reduce or avoid ADCC directed against cells to which the provided antibodies bind. For example, in the case of a provided antibody that includes an IL-15 sequence or a biologically active portion thereof, reduced effector activity of the provided antibody can reduce or prevent ADCC directed against cells, e.g., immune cells, expressing IL2/15Rβ and/or IL2/15Rβ γc receptor subunits to which the provided antibody can bind.

Such antibodies may be useful for the treatment or prevention of a variety of diseases, disorders, or conditions, including inflammatory diseases, disorders, or conditions; autoimmune diseases, disorders, or conditions; metabolic diseases, disorders, or conditions; neoplastic diseases, disorders, or conditions, and cancers. Provided herein in some aspects are methods of using and uses of the provided antibodies for the treatment of a disease or condition, e.g., cancer. These methods can further include the administration of a combination agent, e.g., an anti-tumor agent, in combination with the provided antibody. The combination agent can be one that promotes or mediates ADCC against cells, e.g., tumor cells. In some aspects, the provided antibody has reduced effector function and does not interfere with the ADCC-related effects of the combination agent. Thus, in some aspects, the provided antibodies have an additional advantage in that they can be used in combination therapies without affecting the ability of the combination agent to induce or promote ADCC against tumor cells.

The present disclosure also provides methods and materials for the preparation of the provided chimeric antibodies, including chimeric IL-15 modified antibodies and chimeric IL-2 modified antibodies.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the articles “a” and “an” refer to one or to more than one (i.e. to at least one) of the grammatical object of the article. By way of example, “an element” means one element or more than one element.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

An “ultralong CDR3” or an “ultralong CDR3 sequence”, used interchangeably herein, comprises a CDR3 or CDR3 sequence that is not derived from a human antibody sequence. An ultralong CDR3 may be 35 amino acids in length or longer, for example, 40 amino acids in length or longer, 45 amino acids in length or longer, 50 amino acids in length or longer, 55 amino acids in length or longer, or 60 amino acids in length or longer. Typically, the ultralong CDR3 is a heavy chain CDR3 (CDR-H3 or CDRH3). An ultralong CDR3H3 exhibits features of a CDRH3 of a ruminant (e.g., bovine) sequence. The structure of an ultralong CDR3 includes a “stalk”, composed of ascending and descending strands (e.g. each about 12 amino acids in length), and a disulfide-rich “knob” that sits atop the stalk. The unique “stalk and knob” structure of the ultralong CDR3 results in the two antiparallel β-strands (an ascending and descending stalk strand) supporting a disulfide bonded knob protruding out of the antibody surface to form a mini antigen binding domain. In some embodiments, the ultralong CDR3 antibodies comprise, in order, an ascending stalk region, a knob region, and a descending stalk region. The length of the ultralong CDR3 may include a non-antibody sequence, such as a cytokine sequence, for example IL-15.

A modified ultralong CDR3 refers to an ultralong CDR3 in which at least portion includes a non-antibody sequence, such as a cytokine sequence, for example IL-15. In some cases, at least a portion of the knob of an ultralong CDR3 is replaced or includes the non-antibody sequence.

“Substantially similar,” or “substantially the same”, refers to a sufficiently high degree of similarity between two numeric values (generally one associated with an antibody disclosed herein and the other associated with a reference/comparator antibody) such that one of skill in the art would consider the difference between the two values to be of little or no biological and/or statistical significance within the context of the biological characteristic measured by said values (e.g., Kd values). The difference between said two values is preferably less than about 50%, preferably less than about 40%, preferably less than about 30%, preferably less than about 20%, preferably less than about 10% as a function of the value for the reference/comparator antibody.

“Binding affinity” generally refers to the strength of the sum total of noncovalent interactions between a single binding site of a molecule (e.g., an antibody) and its binding partner (e.g., an antigen). Unless indicated otherwise, “binding affinity” refers to intrinsic binding affinity which reflects a 1:1 interaction between members of a binding pair (e.g., antibody and antigen). The affinity of a molecule X for its partner Y can generally be represented by the dissociation constant. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure.

“Percent (%) amino acid sequence identity” with respect to a peptide or polypeptide sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MegAlign (DNASTAR) software. Those skilled in the art can determine appropriate parameters for measuring alignment, including any algorithms needed to achieve maximal alignment over the full length of the sequences being compared.

“Polypeptide,” “peptide,” “protein,” and “protein fragment” may be used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

“Amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

“Conservatively modified variants” applies to both amino acid and nucleic acid sequences. “Amino acid variants” refers to amino acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated (e.g., naturally contiguous) sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid which encodes a polypeptide is implicit in a described sequence with respect to the expression product, but not with respect to actual probe sequences. As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” including where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles disclosed herein. Typically conservative substitutions include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

“Humanized” or “Human engineered” forms of non-human (e.g., bovine) antibodies are chimeric antibodies that contain amino acids represented in human immunoglobulin sequences, including, for example, wherein minimal sequence is derived from non-human immunoglobulin. For example, humanized or human engineered antibodies may be non-human (e.g., bovine) antibodies in which some residues are substituted by residues from analogous sites in human antibodies (see, e.g., U.S. Pat. No. 5,766,886). A humanized antibody optionally may also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

A “variable domain” with reference to an antibody refers to a specific Ig domain of an antibody heavy or light chain that contains a sequence of amino acids that varies among different antibodies. Each light chain and each heavy chain has one variable region domain (VL, and, VH). The variable domains provide antigen specificity, and thus are responsible for antigen recognition. Each variable region contains CDRs that are part of the antigen binding site domain and framework regions (FRs).

A “constant region domain” refers to a domain in an antibody heavy or light chain that contains a sequence of amino acids that is comparatively more conserved among antibodies than the variable region domain. Each light chain has a single light chain constant region (CL) domain and each heavy chain contains one or more heavy chain constant region (CH) domains, which include, CH1, CH2, CH3 and, in some cases, CH4. Full-length IgA, IgD and IgG isotypes contain CH1, CH2 CH3 and a hinge region, while IgE and IgM contain CH1, CH2 CH3 and CH4. CH1 and CL domains extend the Fab arm of the antibody molecule, thus contributing to the interaction with antigen and rotation of the antibody arms. Antibody constant regions can serve effector functions, such as, but not limited to, clearance of antigens, pathogens and toxins to which the antibody specifically binds, e.g. through interactions with various cells, biomolecules and tissues.

An antibody containing an ultralong CDR3 is an antibody that contains a variable heavy (VH) chain with an ultralong CDR3. An antibody may further include pairing of the VH chain with a variable light (VL) chain. In some embodiments, the antibodies or antigen-binding fragments include a heavy chain variable region and a light chain variable region. Thus, the term antibody include full-length antibodies and portions thereof including antibody fragments, wherein such contain a heavy chain or portion thereof and/or a light chain or portion thereof. An antibody can contain two heavy chains (which can be denoted H and H′) and two light chains (which can be denoted L and L′), in which each L chain is linked to an H chain by a covalent disulfide bond and the the two H chains are linked to each other by disulfide bonds. The terms “full-length antibody,” or “intact antibody” are used interchangeably to refer to an antibody in its substantially intact form, as opposed to an antibody fragment. A full-length antibody is an antibody typically having two full-length heavy chains (e.g., VH-CH1-CH2-CH3 or VH-CH1-CH2-CH3-CH4) and two full-length light chains (VL-CL) and hinge regions.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (V_{H}) regions capable of specifically binding, and single chain variable fragments (scFv).

An “antibody fragment” comprises a portion of an intact antibody, the antigen binding and/or the variable region of the intact antibody. Antibody fragments, include, but are not limited to, Fab fragments, Fab′ fragments, F(ab′)₂ fragments, Fv fragments, disulfide-linked Fvs (dsFv), Fd fragments, Fd′ fragments; single-chain antibody molecules, including single-chain Fvs (scFv) or single-chain Fabs (scFab); antigen-binding fragments of any of the above and multispecific antibodies from from antibody fragments.

A “Fab fragment” is an antibody fragment that results from digestion of a full-length immunoglobulin with papain, or a fragment having the same structure that is produced synthetically, e.g., by recombinant methods. A Fab fragment contains a light chain (containing a V_{L} and C_{L}) and another chain containing a variable domain of a heavy chain (V_{H}) and one constant region domain of the heavy chain (C_{H}1).

An “scFv fragment” refers to an antibody fragment that contains a variable light chain (V_{L}) and variable heavy chain (V_{H}), covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

A chimeric antibody refers to an antibody containing a modified ultralong CDR3 in which at least a portion of a knob of the CDR3 of the heavy chain is replaced or includes a non-antibody sequence, such as a cytokine sequence, for example IL-15.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “effective amount” or “therapeutically effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, “disease or disorder” refers to a pathological condition in an organism resulting from cause or condition including, but not limited to, infections, acquired conditions, genetic conditions, and characterized by identifiable symptoms.

As used herein, the terms “treat,” “treating,” or “treatment” refer to ameliorating a disease or disorder, e.g., slowing or arresting or reducing the development of the disease or disorder, e.g., a root cause of the disorder or at least one of the clinical symptoms thereof.

As used herein, the term “subject” refers to an animal, including a mammal, such as a human being. The term subject and patient can be used interchangeably.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

### II. CHIMERIC ANTIBODIES

Provided herein are chimeric antibodies in which a heterologous sequence, such as a cytokine sequence, e.g., an IL-2 sequence or a biologically active portion thereof or an IL-15 sequence or a biologically active portion thereof, replaces a portion of an ultralong CDR3 region of a heavy chain of a bovine (cow) antibody or a humanized sequence thereof. In some embodiments, the IL-15 sequence may include a full-length IL-15 (e.g., human IL-15) sequence (e.g., the sequence set forth in SEQ ID NO: 1) or a biologically active portion of IL-15. IL-15 is a potent immune stimulatory cytokine and an essential survival factor for T cells and Natural Killer cells. Preclinical studies comparing IL2 and IL15 have shown than IL-15 is associated with less toxicity than IL-2. The IL-15 sequence may also be modified to increase its binding affinity for the IL-15 receptor. For example, the asparagine may be replaced by aspartic acid at position 72 of ILLS (SEQ. ID NO. 2 of US patent publication US20140134128A1; the contents of which are incorporated by reference in their entirety). Any portion of IL-15 that retains one or more functions of full length or mature IL-15 may be useful in the present invention. Such functions include the promotion of NK cell survival, regulation of NK cell and T ceil activation and proliferation as well as the support of NK cell development from hematopoietic stem cells.

In some embodiments, a further portion of the ultralong CDR3 region, e.g., the ascending stalk strand, is modified relative to that of the bovine antibody or humanized sequence thereof. In some embodiments, the heavy chain constant region of the provided chimeric antibodies is modified, e.g., mutated, in order to reduce effector activity of the provided chimeric antibodies, for instance reduced as compared to a wild-type heavy chain constant region. The provided chimeric antibodies also include such antibodies that are linked to or complexed to an extracellular portion of the IL15Rα, such as the IL15Rα sushi domain (e.g., set forth in SEQ ID NO:2). In some embodiments, the IL-15 cytokine is formatted with the alpha subunit of IL15 receptor (IL15Ra) or a portion thereof that binds to and activates membrane-bound IL15 beta/gamma receptor. A unique feature of IL-15 mediated activation is the mechanism of trans-presentation in which IL-15 is presented as a complex with the alpha subunit of IL15 receptor (IL15Ra) that binds to and activates membrane-bound IL15 beta/gamma receptor, either on the same cell or a different cell. The IL15/IL15Ra complex is more effective in activating IL-15 signaling, than IL-15 by itself. In some embodiments, full-length IL-15Ra or a portion of the IL15Ra may be complexed or fused (e.g., linked) to the IL-15 cytokine sequence or the biologically active portion thereof. Any portion of IL-15 and IL-15Ra that retains one or more functions of full length or mature IL15 or IL15Ra respectively may be useful in the provided embodiments. Such functions include the promotion of NK cell survival, regulation of NK cells, and T cell activation and proliferation as well as the support of NK cell development from hematopoietic stem cells. The IL15 receptor alpha comprises an extracellular domain called the sushi domain which contains most of the structural elements necessary for binding to IL15. Thus, in some embodiments, the portion of the IL15Ra is or includes IL15Ra sushi domain. A portion of IL15Ra useful in the provided embodiments may include 31-205 amino acids or 31-95 ammo acids of the human IL15Ra (Uniprot ID: Q1326.1).

The provided antibodies exhibit features of bovine or cow antibodies that have unique heavy chain variable region sequences containing an ultralong CDR3 sequence of up to 70 amino acids or more in length. CDR3 sequence identified in cattle include those designated as: BLV1H12 (see, SEQ ID NO: 25), BLV5B8 (see, SEQ ID NO: 30), BLV5D3 (see, SEQ ID NO: 31), and BLV8C1 1 (see, SEQ ID NO: 32) (see, e.g., Saini, et al. (1999) Eur. Immunol. 29:2420-2426; and Saini and Kaushik (2002) Scand. J. Immunol. 55:140-148); BF4E9 (see, SEQ ID NO: 33) and BF1 H1 (see, SEQ ID NO: 34) (see, e.g., Saini and Kaushik (2002) Scand. J. Immunol. 55:140-148); and F18 (see, SEQ ID NO: 35) (see, e.g., Berens, et al. (1997) Int. Immunol. 9:189-199). Exemplary antibody variable region sequences comprising an ultralong CDR3 sequence identified in cattle include BLV1H12. In some embodiments, the BLV1H12 ultralong CDR3 sequence is encoded by the SEQ ID NO: 25. An exemplary bovine antibody includes bovine antibody BLVH12 (e.g., heavy chain variable region set forth in SEQ ID NO: 26, and light chain variable region set forth in SEQ ID NO: 27); and bovine antibody BLV5B8 (e.g., heavy chain variable region set forth in SEQ ID NO: 28, and light chain variable region set forth in SEQ ID NO: 29).

In cow antibodies, the ultralong CDR3 sequences form a structure where a subdomain with an unusual architecture is formed from a “stalk”, composed of two 12-residue, anti-parallel β-strands (ascending and descending strands), and a 39-residue, disulfide-rich “knob” that sits atop the stalk, far from the canonical antibody paratope. The long anti-parallel β-ribbon serves as a bridge to link the knob domain with the main antibody scaffold. The unique “stalk and knob” structure of the ultralong CDR3 results in the two antiparallel β-strands (an ascending and descending stalk strand) supporting a disulfide bonded knob protruding out of the antibody surface to form a mini antigen binding domain. In some embodiments, the ultralong CDR3 antibodies comprise, in order, an ascending stalk region, a knob region, and a descending stalk region.

The unique “stalk” and knob structural features are conserved across the different bovine or cow ultralong CDR3 sequences. The ascending strand of the stalk comprises mainly hydrophobic side chains and a relatively conserved “T(T/S)VHQ” motif and variants thereof at the base, which initiates the ascending strand. This conserved T(T/S)VHQ motif and variants thereof is typically found following the first cysteine residue in variable region sequences of the various bovine or cow sequences. The conserved T(T/S)VHQ motif is connected by a variable number of residues to a motif (CPDG for BLV1H12) that forms a β-turn at the base of each knob. The stalk can be of variable length, and the descending strand of the stalk comprises alternating aromatics that form a ladder through stacking interactions, that may contribute to the stability of the long solvent-exposed, two stranded β-ribbon (Wang et al. Cell. 2013, 153 (6): 1379-1393).

In some embodiments, the chimeric antibodies provided herein are based on an antibody scaffold that may be derived from or based on a bovine antibody sequence, or a humanized sequence thereof, but include a heterologous sequence, such as a cytokine sequence, e.g., IL-2 sequence or biologically active portion thereof or IL-15 sequence or biologically active portion thereof, that is inserted into or replaces a portion of the knob domain of the ultralong CDR3 of the heavy chain of the bovine antibody sequence or the humanized sequence thereof. In some embodiments, the ultralong CDR3 sequences of the heavy chain of chimeric antibodies provided herein contains a stalk component that contains an ascending strand and descending strand, joined together by a region that contains a heterologous sequence. In some embodiments, the heterologous sequence replaces a portion of, e.g., replaces, the knob region of the bovine antibody or humanized sequence thereof.

In some embodiments, the heterologous sequence is a non-antibody sequence. In some embodiments, the heterologous sequence is a signaling molecule sequence. In some embodiments, the heterologous sequence is a hormone sequence. In some embodiments, the heterologous sequence is a neurotransmitter sequence. In some embodiments, the heterologous sequence is a growth factor. In some embodiments, the heterologous sequence is a cytokine sequence. In some embodiments, the heterologous sequence is a chemokine sequence. In some embodiments, the heterologous sequence is an interferon sequence. In some embodiments, the heterologous sequence is an interleukin sequence. In some embodiments, the heterologous sequence is a lymphokine sequence. In some embodiments, the heterologous sequence is a tumour necrosis factor sequence.

In some embodiments, the heterologous sequence is a cytokine sequence. Thus, the provided chimeric antibodies include chimeric cytokine modified antibodies in which a cytokine sequence replaces all or a portion of the knob region of the bovine antibody or humanized sequence thereof. In some embodiments, the cytokine sequence is an IL-2 sequence or a biologically active portion thereof. In some embodiments, the cytokine sequence is an IL-15 sequence or a biologically active portion thereof.

In some embodiments, the heterologous sequence is inserted into the knob region of the CDR3 sequence of the antibody, including optionally, removing a portion of CDR3 (e.g., one or more amino acids of the CDR3) or the entire CDR3 sequence (e.g., all or substantially all of the amino acids of the CDR3). In some embodiments, the heterologous sequence may be inserted into the knob domain of the ultralong CDR3. In some embodiments, the heterologous sequence is contained between the ascending and descending stalk strands.

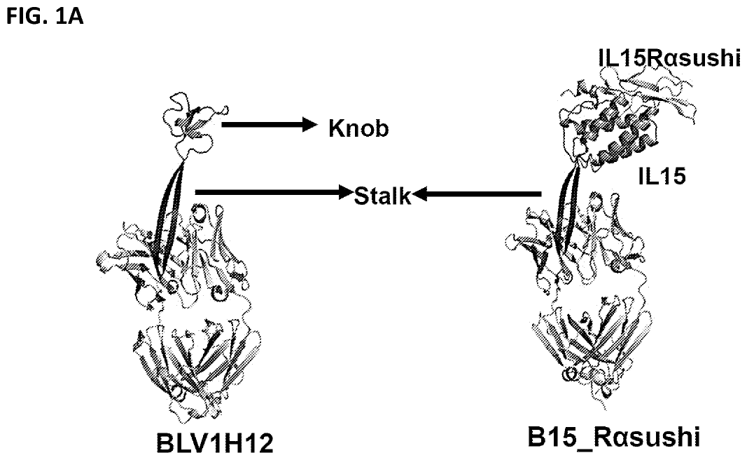

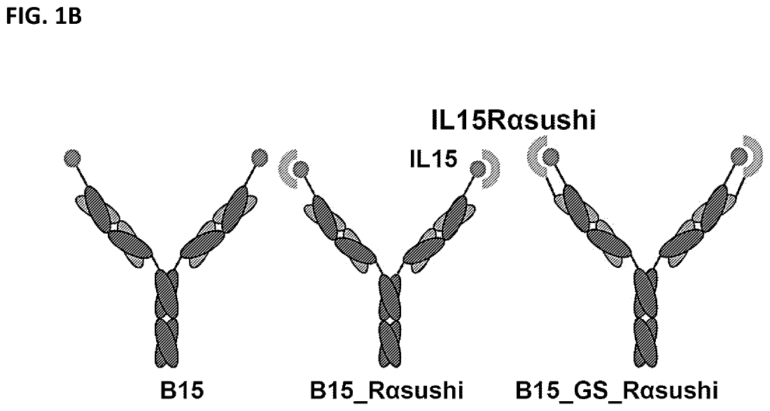

In some embodiments, the IL-15 sequence or a biologically active portion thereof is inserted into the knob region of the CDR3 sequence of the antibody, including optionally, removing a portion of CDR3 (e.g., one or more amino acids of the CDR3) or the entire CDR3 sequence (e.g., all or substantially all of the amino acids of the CDR3). In some embodiments, the IL-15 or biologically active portion thereof may be inserted into the knob domain of the ultralong CDR3 (FIG. 1A and FIG. 1B). In some embodiments, the IL-15 or biologically active portion thereof is contained between the ascending and descending stalk strands.

In some embodiments, the IL-2 sequence or a biologically active portion thereof is inserted into the knob region of the CDR3 sequence of the antibody, including optionally, removing a portion of CDR3 (e.g., one or more amino acids of the CDR3) or the entire CDR3 sequence (e.g., all or substantially all of the amino acids of the CDR3). In some embodiments, the IL-2 or biologically active portion thereof may be inserted into the knob domain of the ultralong CDR3. In some embodiments, the IL-2 or biologically active portion thereof is contained between the ascending and descending stalk strands.

In some embodiments, the ultralong CDR3 may be 35 amino acids in length or more (e.g., 40 or more, 45 or more, 50 or more, 55 or more, 60 or more).

Any of the embodiments provided herein can contain any of the features as described in PCT/US2013/020910, PCT/US2014/047315 or PCT/US2013/020903, all of which are incorporated by reference in their entirety.

Exemplare features of the antibody, including the heavy and light chain, are described in subsections below. In some of any of the embodiments herein, the antibody is a full length or intact antibody. In some of any of the embodiments herein, the antibody is an antigen-binding fragment thereof. In a further embodiment, the antigen-binding fragment thereof is a Fab, Fab′-SH, Fv, scFv, or (Fab′)2 fragment. In some embodiments, the antibody is a Fab.

### A. Heavy Chain Regions

In provided embodiments, the heavy chain of the provided chimeric antibodies is based on or derived from a framework sequence that has an ultralong CDR3, in which a heterologous sequence, such as a cytokine sequence, e.g., IL-2 or a biologically active portion thereof or IL-15 or biologically active portion thereof, is inserted into or replaces at least a portion of the ultralong CDR3 sequence. The antibody framework may be derived from a bovine sequence such as VH-VL, a human germline sequence, or a modified human germline sequence.

In some embodiments, the heavy chain of the provided chimeric antibodies is based on or derived from a bovine or cow framework sequence in which the heterologous sequence, such as the cytokine sequence, e.g. IL-2 sequence or a biologically active portion thereof or IL-15 sequence or biologically active portion thereof, can be inserted into or replace at least a portion of the ultralong CDR3 sequence of a bovine or cow sequence. The antibody may comprise at least a portion of a BLV1H12 antibody containing an ultralong CDR3 fusion containing the heterologous sequence, e.g., cytokine sequence. Alternatively, or additionally, the provided chimeric antibody includes at least a portion of a BLV5D3, BLV8C11, BF1H1, BLV5B8, and/or F18 antibody containing an ultralong CDR3 fusion containing the heterologous sequence, e.g., cytokine sequence. In some embodiments, the heterologous sequence, e.g., the cytokine sequence, such as IL-15 sequence or biologically active portion thereof, can be inserted into or replace at least a portion of the ultralong CDR3 of the sequence set forth in SEQ ID NO: 26 or SEQ ID NO:28.

In some embodiments, the heavy chain of the provided chimeric antibodies is based on or derived from a humanized heavy chain framework sequence that is humanized compared to a bovine or cow sequence. In some embodiments, the heavy chain of the provided chimeric antibodies is based on or derived from a human heavy chain framework sequence that exhibits sequence or structural similarities to a bovine or cow sequence. In some cases, humanization can include engineering an ultralong CDR3 sequence derived from a bovine ultralong CDR3, such as any described above, into a human framework. The human framework may be of germline origin, or may be derived from non-germline (e.g., mutated or affinity matured) sequences. Genetic engineering techniques well known to those in the art, including as disclosed herein, may be used to generate a hybrid DNA sequence containing a human framework and a non-human ultralong CDR3. Unlike human antibodies which may be encoded by V region genes derived from one of seven families, bovine antibodies which produce ultralong CDR3 sequences appear to utilize a single V region family which may be considered to be most homologous to the human VH4 family. In particular embodiments where ultralong CDR3 sequences derived from cattle are to be humanized to produce an antibody comprising an ultralong CDR3, human V region sequences derived from the VH4 family may be genetically fused to a bovine-derived ultralong CDR3 sequence. Exemplary VH4 germline gene sequences in the human antibody locus include VH4-39, VH4-59*03, VH4-34*02, and VH4-34*09 human heavy chain germline sequences. In some embodiments, the human heavy chain germline sequence is a sequence set forth in any one of SEQ ID NOs: 68-71. In some embodiments, the human heavy chain germline sequence is a sequence encoded by the sequence set forth in any one of SEQ ID NOs: 169-172.

In some embodiments, the heterologous sequence, such as the cytokine sequence, such as IL-2 sequence or a biologically active portion thereof or IL-15 sequence or biologically active portion thereof, can be inserted into or replace at least a portion of the ultralong CDR3 of a human germline sequence comprising the sequence set forth in SEQ ID NOs: 68-71.

In some embodiments, the provided chimeric antibodies include a fusion of a human VH4 framework sequence to a bovine-derived ultralong CDR3 into which at least a portion of the knob is replaced with the heterologous sequence, e.g., IL-15 or IL-2 sequence or a biologically active portion thereof. In some aspects, such fusions can be generated through the following steps. First, the second cysteine of a V region genetic sequence is identified along with the nucleotide sequence encoding the second cysteine. Generally, the second cysteine marks the boundary of the framework and CDR3 two residues upstream (N-terminal) of the CDR3. Second, the second cysteine in a bovine-derived V region sequence is identified which similarly marks 2 residues upstream (N-terminal) of the CDR3. Third, the genetic material encoding the human V region is combined with the genetic sequence encoding the ultralong CDR3. Thus, a genetic fusion may be made, wherein the ultralong CDR3 sequence is placed in frame of the human V region sequence. Preferably a humanized antibody comprising an ultralong CDR3 is as near to human in amino acid composition as possible. Optionally, a J region sequence may be mutated from a bovine-derived sequence to a human sequence. Also optionally, a humanized heavy chain may be paired with a human light chain.

In some embodiments, the modified VH region of the provided chimeric antibodies is a variant of the VH region of a bovine antibody, e.g., BLV1H12. In some embodiments, the modified VH region of the provided chimeric antibodies is a variant of a humanized sequence of the VH region of a bovine antibody, e.g., BLV1H12.

In some embodiments, the provided chimeric antibody or binding fragment thereof comprises a heavy chain variable region comprising a sequence of the formula V1-X-V2, wherein the V1 region of the heavy chain comprises a heavy chain sequence portion containing three framework regions (e.g., FR-1, FR-2, and FR-3) separating two CDR regions (CDR1 and CDR2); the X region comprises a modified ultralong CDR3 sequence, which can include the heterologous sequence, e.g., an IL-2 sequence or a biologically active portion thereof or an IL-15 sequence or a biologically active portion thereof; and the V2 region comprises a portion of the heavy chain including FR-4.

In some embodiments, the V1 region comprises the formula FR1-CDR1-FR2-CDR2-FR3. In some embodiments, the V1 region comprises an amino acid sequence selected from the group consisting of: (i) bovine heavy chain regions comprising amino acids of SEQ ID NO: 26 (encoded by the nucleotide of SEQ ID NO:5), or (i) a humanized heavy chain regions comprising human germline variable regions comprising SEQ ID NOS: 12-19. In some embodiments, the V1 region comprises the sequence set forth in SEQ ID NO: 182 or SEQ ID NO: 197.

In some embodiments, the modified VH region of the provided chimeric antibodies is a variant of the VH region of a bovine antibody, e.g., BLV1H12. In some embodiments, the V1 region comprises the sequence set forth in SEQ ID NO: 182.

In some embodiments, the modified VH region of the provided chimeric antibodies is a variant of a humanized sequence of the VH region of a bovine antibody, e.g., BLV1H12. In some embodiments, the V1 region comprises the sequence set forth in SEQ ID NO: 197, or a sequence that exhibits at least at or about 65%, at least at or about 70%, at least at or about 75%, at least at or about 80%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the amino acid sequence set forth in SEQ ID NO:197. In some embodiments, the V1 region comprises the sequence set forth in SEQ ID NO: 197.

In some embodiments, the X region comprises the modified ultralong CDR3 sequence, which can include a heterologous sequence, e.g., an IL-15 sequence or a biologically active portion thereof (e.g., a human IL-15 sequence or a biologically active portion thereof). In some embodiments, the IL-15 sequence comprises the amino acid sequence set forth in SEQ ID NO: 1 or a sequence of amino acids that exhibits at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the amino acid sequence set forth in SEQ ID NO:1. In some embodiments, the IL-15 sequence comprises the amino acid sequence found in SEQ ID NO: 1.

In some embodiments, the IL-15 sequence exhibits activity to stimulate the proliferation, activation, or cytotoxicity of cytotoxic T lymphocytes and natural killer (NK) cells, such as in an in vitro assay or in vivo. In some embodiments, the IL-15 sequence exhibits binding to IL2/15Rβ and/or γc subunits, such as in an in vitro binding assay. In some embodiments, the activity or binding is similar to or retained compared to a recombinant IL-15 monomer.

In some embodiments, the heterologous sequence, e.g., the IL-15 sequence or biologically active portion thereof, is inserted into or replaces a portion of the knob of the ultralong CDR3 between the ascending and descending stalk regions. The heterologous sequence, e.g., the IL-15 sequence, may be positioned between the stalk regions, in which the heterologous sequence, e.g., the IL-15 sequence, is linked directly or indirectly to each of the stalk regions. In some embodiments, the linkage to one or both of the stalk sequences is indirect via a linker. The linker can comprise an amino acid sequence of (GGGGS), wherein n=1 to 5. Alternatively, the linker comprises an amino acids sequence of (GSG)n, GGGSGGGGS or GGGGSGGGS. In some cases, the linker has the sequence GGS (SEQ ID NO: 151) or GSG (SEQ ID NO: 186).

In some embodiments, the X region comprises the modified ultralong CDR3 sequence, which can include a heterologous sequence, e.g., an IL-2 sequence or a biologically active portion thereof (e.g., a human IL-2 sequence or a biologically active portion thereof). In some embodiments, the IL-2 sequence comprises the amino acid sequence set forth in SEQ ID NO: 165 or a sequence of amino acids that exhibits at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the amino acid sequence set forth in SEQ ID NO:165. In some embodiments, the IL-2 sequence comprises the amino acid sequence found in SEQ ID NO: 165.

In some embodiments, the IL-2 sequence exhibits activity to stimulate the proliferation, activation, or cytotoxicity of cytotoxic T lymphocytes and natural killer (NK) cells, such as in an in vitro assay or in vivo. In some embodiments, the IL-2 sequence exhibits binding to IL2/15Rβ and/or γc subunits, such as in an in vitro binding assay. In some embodiments, the activity or binding is similar to or retained compared to a recombinant IL-2 monomer.

In some embodiments, the heterologous sequence, e.g., the IL-2 sequence or biologically active portion thereof, is inserted into or replaces a portion of the knob of the ultralong CDR3 between the ascending and descending stalk regions. The heterologous sequence, e.g., the IL-2 sequence, may be positioned between the stalk regions, in which the heterologous sequence, e.g., the IL-2 sequence, is linked directly or indirectly to each of the stalk regions. In some embodiments, the linkage to one or both of the stalk sequences is indirect via a linker. The linker can comprise an amino acid sequence of (GGGGS), wherein n=1 to 5. Alternatively, the linker comprises an amino acids sequence of (GSG)n, GGGSGGGGS or GGGGSGGGS. In some cases, the linker has the sequence GGS (SEQ ID NO: 151) or GSG (SEQ ID NO: 186).

The ultralong CDR3 may comprise at least a portion of a knob domain of a CDR3, at least a portion of a stalk domain of a CDR3, or a combination thereof. The portion of the knob domain of the CDR3 may comprise one or more conserved motifs derived from the knob domain of the ultralong CDR3. The stalk domain of the CDR3 may comprise one or more conserved motifs derived from the stalk domain of the ultralong CDR3.

In aspects of each or any of the above or below mentioned embodiments, the ultralong CDR3 is 35 amino acids in length or longer, 40 amino acids in length or longer, 45 amino acids in length or longer, 50 amino acids in length or longer, 55 amino acids in length or longer, or 60 amino acids in length or longer. In some embodiments of each or any of the above or below mentioned embodiments, the ultralong CDR3 is 35 amino acids in length or longer.

In some embodiments, the X region of the provided chimeric antibodies includes an ascending stalk strand and a descending stalk strand. In some embodiments, the heterologous sequence of the provided chimeric antibodies, such as the cytokine sequence, e.g., the IL-15 sequence, is between the ascending stalk strand and the descending stalk strand. In some embodiments, the provided chimeric antibodies include the ascending stalk strand and the descending stalk strand of the bovine antibody or humanized sequence thereof, e.g., that of BLV1H12 or a humanized sequence thereof. In some embodiments, one or both of the ascending and descending stalk strands is a variant of the ascending or descending stalk strand of the bovine antibody or humanized sequence thereof. In some embodiments, the ascending stalk strand of the provided chimeric antibodies is a variant of the ascending stalk strand of the bovine antibody or humanized sequence thereof.

In some embodiments, the X region of the provided chimeric antibodies includes the motif X¹X²X³X⁴X⁵-[heterologous sequence]-(X^{a}X^{b})z motif. In some embodiments, the ultralong CDR3 is 45 amino acids in length or longer. In some embodiments one or more additional amino acids may be present between the X¹X²X³X⁴X⁵ motif and the heterologous sequence and/or between the (X^{a}X^{b})z motif and the heterologous sequence.

In some embodiments, the X¹X²X³X⁴X⁵ motif is all or a portion of the ascending stalk strand. In some embodiments, the X¹X²X³X⁴X⁵ motif on the ascending stalk strand comprises a sequence selected from TTVHQ (SEQ ID NO: 36), TSVHQ (SEQ ID NO: 37) or any one of SEQ ID NOs: 38-67. In some embodiments, the ascending stalk strand comprises a sequence selected from SEQ ID NOs: 72-75 or SEQ ID NO:158. In some embodiments, the ultralong CDR3 comprises an ascending stalk region encoded by SEQ ID NO: 9, SEQ ID NO: 81-121 or SEQ ID NO:157. In some embodiments, the motif includes an N-terminal cysteine (Cys or C) residue, such as set forth a CX¹X²X³X⁴X⁵. For example, in some cases, an ascending stalk region encoded by any of SEQ ID NOs: 36-67, 72-75 or SEQ ID NO:158 may additionally contain an N-terminal Cys residue. Such an exemplary ascending stalk region is set forth in SEQ ID NO:159. In some embodiments, the ascending stalk region of the provided chimeric antibodies includes the sequence set forth in SEQ ID NO: 159. In some embodiments, the ascending stalk strand further comprises the sequence ETKKYQT. In some embodiments, the ascending stalk strand further comprises the sequence ETKKYQS.

In some embodiments, the ascending stalk strand comprises the sequence CX₂TVX₅QETKKYQT. In some embodiments, X₂ and X₅ are any amino acid. In some embodiments, X₂ is Ser, Thr, Gly, Asn, Ala, or Pro. In some embodiments, X₅ is His, Gln, Arg, Lys, Gly, Thr, Tyr, Phe, Trp, Met, Ile, Val, or Leu. In some embodiments, X₂ is Ser, Thr, Gly, Asn, Ala, or Pro, and X₅ is His, Gln, Arg, Lys, Gly, Thr, Tyr, Phe, Trp, Met, Ile, Val, or Leu. In some embodiments, X₂ is Ser, Ala, or Thr. In some embodiments, X₅ is His or Tyr. In some embodiments, X₂ is Ser, Ala, or Thr, and X₅ is His or Tyr. In some embodiments, X₂ is Ser, and X₅ is His. In some embodiments, X₂ is Ala, and X₅ is His. In some embodiments, X₂ is Thr, and X₅ is Tyr. In some embodiments, the ascending stalk region of the provided chimeric antibodies includes the sequence set forth in any of SEQ ID NOs: 183-185. In some embodiments, the ascending stalk region of the provided chimeric antibodies includes the sequence set forth in SEQ ID NO: 183. In some embodiments, the ascending stalk region of the provided chimeric antibodies includes the sequence set forth in SEQ ID NO: 184. In some embodiments, the ascending stalk region of the provided chimeric antibodies includes the sequence set forth in SEQ ID NO: 185.

In some embodiments, the (X^{a}X^{b})z motif is a portion of the descending stalk strand, wherein X^{a} is any amino acid residue, X^{b} is an aromatic amino acid selected from the group consisting of: tyrosine (Y), phenylalanine (F), tryptophan (W), and histidine (H), and wherein z is 1-4. In some embodiments, the descending stalk strand comprises alternating aromatics with the formula YXYXYX where is X is any amino acid. In some embodiments, the descending stalk strand comprises a sequence contained in SEQ ID NO: 76-80 or SEQ ID NO:161. In some embodiments, the ultralong CDR3 comprises a descending stalk region encoded by SEQ ID NO: 122-149 or SEQ ID NO:160. In some embodiments, the descending stalk region of the provided chimeric antibodies includes the sequence set forth in SEQ ID NO: 10.

In some embodiments, the provided chimeric antibodies include a modified ultralong CDR3.

In some embodiments, the modified ultralong CDR3 comprises, in order an ascending stalk region having an amino acid sequence encoded by SEQ ID NO:9, an IL15 cytokine sequence set forth by SEQ ID NO:1, and a descending stalk region having an amino acid sequence encoded by SEQ ID NO: 10. In some embodiments, the ultralong CDR3 comprises, in order an ascending stalk region having an amino acid sequence encoded by SEQ ID NO: 157, an IL15 cytokine sequence set forth by SEQ ID NO:1, and a descending stalk region having an amino acid sequence encoded by SEQ ID NO: 160.

In some embodiments, the modified ultralong CDR3 comprises, in order an ascending stalk region having an amino acid sequence encoded by SEQ ID NO:9, an IL2 cytokine sequence set forth by SEQ ID NO: 165, and a descending stalk region having an amino acid sequence encoded by SEQ ID NO: 10. In some embodiments, the ultralong CDR3 comprises, in order an ascending stalk region having an amino acid sequence encoded by SEQ ID NO: 157, an IL2 cytokine sequence set forth by SEQ ID NO:165, and a descending stalk region having an amino acid sequence encoded by SEQ ID NO: 160.

In some embodiments, the modified ultralong CDR3 comprises, in order, an ascending stalk strand having an amino acid sequence set forth by SEQ ID NO: 183, an IL-15 cytokine sequence set forth by SEQ ID NO: 1, and a descending stalk strand having an amino acid sequence set forth by SEQ ID NO: 10. In some embodiments, the modified ultralong CDR3 comprises the sequence set forth in SEQ ID NO: 206.

In some embodiments, the modified ultralong CDR3 comprises, in order, an ascending stalk strand having an amino acid sequence set forth by SEQ ID NO: 184, an IL-15 cytokine sequence set forth by SEQ ID NO: 1, and a descending stalk strand having an amino acid sequence set forth by SEQ ID NO: 10. In some embodiments, the modified ultralong CDR3 comprises the sequence set forth in SEQ ID NO: 207.

In some embodiments, the modified ultralong CDR3 comprises, in order, an ascending stalk strand having an amino acid sequence set forth by SEQ ID NO: 185, an IL-15 cytokine sequence set forth by SEQ ID NO: 1, and a descending stalk strand having an amino acid sequence set forth by SEQ ID NO: 10. In some embodiments, the modified ultralong CDR3 comprises the sequence set forth in SEQ ID NO: 208.

In some embodiments, the modified ultralong CDR3 comprises, in order, an ascending stalk strand having an amino acid sequence set forth by SEQ ID NO: 159, an IL-15 cytokine sequence set forth by SEQ ID NO: 1, and a descending stalk strand having an amino acid sequence set forth by SEQ ID NO: 10. In some embodiments, the modified ultralong CDR3 comprises the sequence set forth in SEQ ID NO: 209.

In some embodiments, the V2 region of the heavy chain comprises an amino acid sequence selected from the group consisting of (i) WGHGTAVTVSS (SEQ ID NO: 20), (ii) WGKGTTVTVSS (SEQ ID NO: 21), (iii) WGKGTTVTVSS (SEQ ID NO: 22), (iv) WGRGTLVTVSS (SEQ ID NO: 23), (v) WGKGTTVTVSS (SEQ ID NO: 24), and (vi) WGQGLLVTVSS (SEQ ID NO: 11). In some embodiments, the V2 region of the heavy chain comprises the sequence set forth in SEQ ID NO: 11.

In some embodiments, the modified VH region of the provided chimeric antibodies is a variant of the VH region of a bovine antibody, e.g., BLV1H12.

In some embodiments, the heavy chain comprises the formula V1-X-V2-C, wherein the V1 region of the heavy chain comprises the sequence set forth in SEQ ID NO:182; the X region comprises the modified ultralong CDR3 sequence; the V2 region comprises the sequence set forth in SEQ ID NO: 11; and the C region comprises an immunoglobulin constant region, such as a modified IgG (e.g., IgG1) constant region as described. In some embodiments, the X comprises the sequence set forth in any of SEQ ID NOs: 206-208. In some embodiments, the modified VH region comprises the sequence set forth in SEQ ID NO: 200, or a sequence that exhibits at least at or about 85%, a at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the sequence set forth in SEQ ID NO: 200. In some embodiments, the modified VH region comprises the sequence set forth in SEQ ID NO: 200. In some embodiments, the modified VH region comprises the sequence set forth in SEQ ID NO: 201, or a sequence that exhibits at least at or about 85%, a at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the sequence set forth in SEQ ID NO: 201. In some embodiments, the modified VH region comprises the sequence set forth in SEQ ID NO: 201. In some embodiments, the modified VH region comprises the sequence set forth in SEQ ID NO: 202, or a sequence that exhibits at least at or about 85%, a at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the sequence set forth in SEQ ID NO: 202. In some embodiments, the modified VH region comprises the sequence set forth in SEQ ID NO: 202.

In some embodiments, the modified VH region of the provided chimeric antibodies is a variant of a humanized sequence of the VH region of a bovine antibody, e.g., BLV1H12. In some embodiments, the heavy chain comprises the formula V1-X-V2-C, wherein the X region comprises the modified ultralong CDR3 sequence; the V2 region comprises the sequence set forth in SEQ ID NO:11; and the C region comprises an immunoglobulin constant region, such as a modified IgG (e.g., IgG1) constant region as described. In some embodiments, the V1 region of the heavy chain comprises the sequence set forth in SEQ ID NO: 197, or a sequence that exhibits at least at or about 65%, at least at or about 70%, at least at or about 75%, at least at or about 80%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the amino acid sequence set forth in SEQ ID NO: 197. In some embodiments, the V1 region comprises the sequence set forth in SEQ ID NO: 197. In some embodiments, the X comprises the sequence set forth in any of SEQ ID NOs: 206-208. In some embodiments, the modified VH region comprises the sequence set forth in SEQ ID NO: 203, or a sequence that exhibits at least at or about 85%, a at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the sequence set forth in SEQ ID NO: 203. In some embodiments, the modified VH region comprises the sequence set forth in SEQ ID NO: 203. In some embodiments, the modified VH region comprises the sequence set forth in SEQ ID NO: 204, or a sequence that exhibits at least at or about 85%, a at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the sequence set forth in SEQ ID NO: 204. In some embodiments, the modified VH region comprises the sequence set forth in SEQ ID NO: 204. In some embodiments, the modified VH region comprises the sequence set forth in SEQ ID NO: 205, or a sequence that exhibits at least at or about 85%, a at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the sequence set forth in SEQ ID NO: 205. In some embodiments, the modified VH region comprises the sequence set forth in SEQ ID NO: 205.

In particular embodiments, a chimeric IL-15 modified antibody or antigen-binding fragment provided herein contains a variable heavy chain sequence encoded by the sequence of nucleotides set forth in SEQ ID NO:7 or a sequence of nucleotides that exhibits at least at or about 85%, a at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the nucleotide sequence set forth in SEQ ID NO:7, in which is contained a modified ultralong CDR3 containing an IL-15 sequence. In some embodiments, the chimeric IL-15 modified antibody or antigen-binding fragment provided herein comprises a variable heavy chain sequence encoded by the sequence of nucleotides set forth in SEQ ID NO:7. In some embodiments, the chimeric IL-15 modified antibody or antigen-binding fragment provided herein consists of or consists essentially of a variable heavy chain sequence encoded by the sequence of nucleotides set forth in SEQ ID NO:7.

In some embodiments, the heavy chain includes a variable heavy chain as described that is joined to a human constant region. In some embodiments, the human constant region includes the CH1-CH2-CH3 constant domains. In some embodiments, the human constant region is of human IgG1 (e.g., with the sequence set forth in SEQ ID NO: 196, or a naturally occurring variant thereof, for instance with the K97R, D239E, or L241M mutation).

In some embodiments, the human IgG is human IgG1 (e.g., with the sequence set forth in SEQ ID NO: 196, or a naturally occurring variant thereof, for instance with the K97R, D239E, or L241M mutation).

In some embodiments, the heavy chain constant region is mutated or modified, i.e., is a modified constant region. In some embodiments, the heavy chain constant region is a modified human IgG heavy chain constant region. In some cases, the mutations include one or more amino acid substitutions to reduce effector activity of the heavy chain constant region. In some embodiments, the heavy chain constant region is modified to reduce effector activity of the antibody. In some embodiments, the modified human IgG heavy chain constant region has reduced effector activity. In some embodiments, effector activity is reduced compared to a wild-type human IgG heavy chain constant region. In some of any of the described embodiments, the modified human IgG heavy chain constant region is modified compared to the constant region of wildtype human IgG1. In some embodiments, the modified human IgG heavy chain constant region has a sequence that is modified by one or more amino acid substitutions compared to SEQ ID NO: 196 (or a naturally occurring variant thereof, e.g. with K97R, D239E or L241M mutations) and that exhibits at least 85%, at least 90%, at least 95% or at least 98% sequence identity to SEQ ID NO: 196 or the natural variant thereof and contains the one or more amino acid substitutions, for example to reduce effector activity of the heavy chain constant region.

Various examples of mutations to heavy chain constant regions to alter, such as reduce, effector function are known, including any as described below. In some embodiments, reference to amino acid substitutions in a heavy chain constant region is by EU numbering by Kabat (also called Kabat numbering) unless described with reference to a specific SEQ ID NO. EU numbering is known and is according to the most recently updated IMGT Scientific Chart (IMGT®, the international ImMunoGeneTics information System®, http://www.imgt.org/IMGTScientificChart/Numbering/Hu_IGHGnber.html (created: 17 May 2001, last updated: 10 Jan. 2013) and the EU index as reported in Kabat, E. A. et al. Sequences of Proteins of Immunological interest. 5th ed. US Department of Health and Human Services, NIH publication No. 91-3242 (1991).

In some embodiments, a modified heavy chain constant region that exhibits reduced effector functions may be a desirable candidate for applications in which binding of the chimeric antibody to a cell surface target, e.g., the binding of an IL-15 sequence to IL-15 receptor subunits, is desired yet certain effector functions, such as complement-dependent cytotoxicity (CDC) and antibody-dependent cell cytotoxicity (ADCC), are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the provided chimeric antibodies lack FcγR binding (hence likely lacking ADCC activity). In some embodiments, the provided chimeric antibodies lack FcγR binding and retain FcRn binding ability. The primary cells for mediating ADCC, NK cells, express FcγRIII only, whereas monocytes express FcγRI, FcγRII and FcγRIII. Non-limiting examples of in vitro assays to assess ADCC activity of a molecule of interest are described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the multispecific polypeptide construct or cleaved components thereof is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

In some embodiments, the heavy chain constant region is modified to alter antibody-dependent cellular cytotoxicity (ADCC) and/or complement-dependent cytotoxicity (CDC), e.g., the amino acid modifications described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48:152-164; Alegre et al, 1992 J Immunol, 148:3461-3468; Reviewed in Kaneko and Niwa, 2011 Biodrugs, 25(1):1-11.

In some embodiments, the heavy chain constant region is altered at one or more of the following positions to reduce Fc receptor binding: Leu 234 (L234), Leu235 (L235), Asp265 (D265), Asp270 (D270), Ser298 (S298), Asn297 (N297), Asn325 (N325), Ala327 (A327) or Pro329 (P329). For example, Leu 234Ala (L234A), Leu235Ala (L235A), Leu235Glu (L235E), Asp265Asn (D265N), Asp265Ala (D265A), Asp270Asn (D270N), Ser298Asn (S298N), Asn297Ala (N297A), Pro329Ala (P329A) or Pro239Gly (P329G), Asn325Glu (N325E) orAla327Ser (A327S). In some embodiments, modifications within the heavy chain constant region reduce binding to Fc-receptor-gamma receptors while have minimal impact on binding to the neonatal Fc receptor (FcRn).

In some embodiments, the heavy chain constant region is modified at amino acid Asn297 (Kabat Numbering) to prevent glycosylation of the chimeric antibody, e.g., Asn297Ala (N297A) or Asn297Asp (N297D). In some embodiments, the heavy chain constant region is modified at amino acid Leu235 (Kabat Numbering) to alter Fc receptor interactions, e.g., Leu235Glu (L235E) or Leu235Ala (L235A). In some embodiments, the heavy chain constant region of the chimeric antibody is modified at amino acid Leu234 (Kabat Numbering) to alter Fc receptor interactions, e.g., Leu234Ala (L234A). In some embodiments, the heavy chain constant region of the chimeric antibody is modified at amino acid Leu234 (Kabat Numbering) to alter Fc receptor interactions, e.g., Leu235Glu (L235E). In some embodiments, the heavy chain constant region of the chimeric antibody is altered at both amino acids 234 and 235, e.g., Leu234Ala and Leu235Ala (L234A/L235A) or Leu234Val and Leu235Ala (L234V/L235A). In some embodiments, the modified heavy chain constant region comprises Leu234Ala and Leu235Ala (L234A/L235A) mutations. In some embodiments, the heavy chain constant region of the chimeric antibody is altered at amino acids 234, 235, and 297, e.g., Leu234Ala, Leu235Ala, Asn297Ala (L234A/L235A/N297A). In some embodiments, the heavy chain constant region of the chimeric antibody is altered at amino acids at 234, 235, and 329, e.g., Leu234Ala, Leu235Ala, Pro239Ala (L234A/L235A/P329A). In some embodiments, the heavy chain constant region of the chimeric antibody is modified at amino acid Asp265 (Kabat Numbering) to alter Fc receptor interactions, e.g. Asp265Ala (D265A). In some embodiments, the heavy chain constant region of the chimeric antibody is modified at amino acid Pro329 (Kabat Numbering) to alter Fc receptor interactions, e.g. Pro329Ala (P329A) or Pro329Gly (P329G). In some embodiments, the heavy chain constant region of the chimeric antibody is altered at both amino acids 265 and 329, e.g., Asp265Ala and Pro329Ala (D265A/P329A) or Asp265Ala and Pro329Gly (D265A/P329G). In some embodiments, the heavy chain constant region of the chimeric antibody is altered at amino acids at 234, 235, and 265, e.g., Leu234Ala, Leu235Ala, Asp265Ala (L234A/L235A/D265A). In some embodiments, the heavy chain constant region of the chimeric antibody is altered at amino acids at 234, 235, and 329, e.g., Leu234Ala, Leu235Ala, Pro329Gly (L234A/L235A/P329G). In some embodiments, the heavy chain constant region of the chimeric antibody is altered at amino acids at 234, 235, 265 and 329, e.g., Leu234Ala, Leu235Ala, Asp265Ala, Pro329Gly (L234A/L235A/D265A/P329G). In some embodiments, the heavy chain constant region of the chimeric antibody is altered at Gly235 to reduce Fc receptor binding. For example, wherein Gly235 is deleted from the heavy chain constant region of the chimeric antibody. In some embodiments, the heavy chain constant region of the chimeric antibody is modified at amino acid Gly236 to enhance the interaction with CD32A, e.g., Gly236Ala (G236A). In some embodiments, the heavy chain constant region of the chimeric antibody lacks Lys447 (EU index of Kabat et al 1991 Sequences of Proteins of Immunological Interest).

In some embodiments, the heavy chain constant region of the chimeric antibody is lacking an amino acid at one or more of the following positions to reduce Fc receptor binding: Glu233 (E233), Leu234 (L234), or Leu235 (L235). In some embodiments, the heavy chain constant region of the chimeric antibody is lacking an amino acid at one or more of the following positions Glu233 (E233), Leu234 (L234), or Leu235 (L235), and is modified at one or more of Asp265 (D265), Asn297 (N297), or Pro329 (P329), to reduce Fc receptor binding. In some embodiments, the heavy chain constant region of the chimeric antibody comprises a three amino acid deletion in the lower hinge corresponding to IgG1 E233, L234, and L235. In some aspects, such heavy chain constant regions s do not engage FcγRs and thus are referred to as “effector silent” or “effector null.”

In some embodiments, the modified heavy chain constant region includes Leu234Ala and Leu235Ala (L234A/L235A) mutations. In some embodiments, the modified heavy chain constant region includes the sequence set forth in SEQ ID NO: 187, or includes a sequence with reduced effector activity that exhibits at least at or about 85%, a at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the sequence set forth in SEQ ID NO: 187. In some embodiments, the modified VH region comprises the sequence set forth in SEQ ID NO: 187. In some embodiments, the modified heavy chain constant region includes the sequence set forth in SEQ ID NO: 188, or includes a sequence with reduced effector activity that exhibits at least at or about 85%, a at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the sequence set forth in SEQ ID NO: 188. In some embodiments, the modified VH region comprises the sequence set forth in SEQ ID NO: 188.

### B. Light Chain Regions

In some embodiments, the provided chimeric antibody or antigen binding fragment further comprises a light chain variable region. In some embodiments, the chimeric antibody variable heavy region or heavy chain is based on a bovine sequence and is paired with a variable light region or light chain of a bovine antibody. In some embodiments, the chimeric antibody variable heavy region or heavy chain is based on a humanized sequence and is paired with a variable light region or light chain of a bovine antibody. In some embodiments, the chimeric antibody variable heavy region or heavy chain is based on a humanized sequence and is paired with a humanized variable light region or light chain of a bovine antibody. In some embodiments, the light chain is a lambda light chain.

In some embodiments, the variable light region is a variable light region of a bovine antibody, such as a variable light region of BLVH12, BLV5D3, BLV8C11, BF1H1, BLV5B8 and/or F18. In some embodiments, the light chain variable region may comprise a sequence based or derived from the polypeptide sequence of SEQ ID NO: 27 or 29. In some embodiments, the light chain polypeptide sequence is encoded by a DNA sequence based on or derived from the DNA sequence of SEQ ID NO:8. In some embodiments, the light chain polypeptide sequence is encoded by a DNA sequence based on or derived from the DNA sequence of SEQ ID NO: 168.

In some embodiments, the light chain includes a variable light region of a bovine antibody that is joined to a human lambda light chain constant region (e.g., set forth in SEQ ID NO: 155). In some embodiments, a portion of the BLV1H12 light chain variable region (e.g., set forth in SEQ ID NO:8 or SEQ ID NO:168) is joined with the human lambda light chain constant region.

In some embodiments, the light chain is a humanized light chain or is a human light chain. In embodiments, the present disclosure provides pairing of a humanized heavy chain comprising an ultralong CDR3 with a human light chain. In some embodiments, the light chain is homologous to a bovine light chain known to pair with a bovine ultralong CDR3 heavy chain. Several human VL sequences can be used to paired with the sequences above, including VL1-47, VL1-40, VL1-51, and VL2-18, which are homologous to the lambda region derived from Bos taurus. In some embodiments, the light chain variable region is a sequence set forth in any one of SEQ ID NOS: 156 or 173-176. In some embodiments, the light chain variable sequence is a sequence encoded by the sequence set forth in any one of SEQ ID Nos: 177-180. In some embodiments, the light chain variable region comprises a variable region of the VL1-51 germline sequence set forth in SEQ ID NO: 156.

In some embodiments, the light chain variable region is a human germline light chain sequence, such as any described above, that contains one or more amino acid modifications. Such modifications may include the substitution of certain amino acid residues in the human light chain to those residues at corresponding positions in a bovine light chain sequence. The modified light chains may improve the yield of the antibody comprising the ultralong CDR3 and/or increase its binding specificity. In some embodiments, the modifications include one or more of amino acid replacements S2A, T5N, P8S, A12G, A13S, and P14L based on Kabat numbering. In some embodiments, the modifications include amino acid replacements S2A, T5N, P8S, A12G, A13S, and P14L based on Kabat numbering. In some embodiments, the modifications are in the CDR1 and include amino acid replacements 129V and N32G. In some embodiments, the modifications are in the CDR2 and include substitution of DNN to GDT. In some embodiments, the modifications are inn CDR2 and include a substitution DNNKRP to GDTSRA. In some embodiments, the modifications include a combination of any of the foregoing. For example, provided modifications of a human germline light chain sequence include amino acid replacements S2A, T5N, P8S, A12G, A13S, and P14L based on Kabat numbering and substitution of DNN to GDT in CDR2.

In some embodiments, the light chain includes a humanized variable light chain as described that is joined to a human lambda light chain constant region (e.g., set forth in SEQ ID NO: 155). In some embodiments, a portion of the light chain variable region, such as a modified human germline light chain, is joined with the human lambda light chain constant region.

In some embodiments, the light chain of the provided chimeric antibodies is a humanized light chain. In some embodiments, the light chain comprises the amino acid sequence set forth in SEQ ID NO:181 or a sequence of amino acids that exhibits at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99% sequence identity to the amino acid sequence set forth in SEQ ID NO:181. In some embodiments, the light chain comprises the sequence set forth in SEQ ID NO: 181. In some embodiments, the sequence of the light chain is set forth in SEQ ID NO: 181.

### C. IL-15RA Sushi Domain

In some embodiments, the chimeric cytokine antibodies containing an IL-15 sequence or biologically active portion thereof as provided herein can further be linked or complexed with all or a portion of the IL-15 high affinity receptor α (IL15Rα) receptor subunit, such as a portion containing an extracellular domain of IL15Rα. In some embodiments, the all or portion of IL15Rα is linked or complexed to the provided chimeric antibodies in order to increase trans signaling to the receptor β and γ subunits (IL2/15Rβ and γc) receptor subunits.

In some embodiments, the provided chimeric antibodies are linked or complexed with a portion of the extracellular domain of IL15Rα. In some embodiments, the provided chimeric antibodies are linked or complexed with the IL15Rα sushi domain. In some embodiments, the IL15Rα sushi domain comprises the sequence set forth in SEQ ID NO: 2.

In some embodiments, provided herein is a chimeric IL-15 modified antibody or antigen-binding fragment in which the heavy chain or variable sequence thereof includes an IL-15 sequence or biologically active portion thereof that replaces all or a portion of the knob region of an ultralong CDR3 of a bovine antibody or humanized sequence thereof (e.g., the IL-15 sequence is placed between the ascending and descending stalk of the ultralong CDR3) that is linked or complexed with an extracellular domain of IL15Rα, such as the IL15Rα sushi domain. In some embodiments, the chimeric IL-15 modified antibody or antigen-binding fragment is complexed with the IL15Rα sushi domain set forth in SEQ ID NO: 2.

In some embodiments, the chimeric antibody can be generated by co-expressing all or a portion of the IL15Rα extracellular domain, e.g., the IL15Rα sushi domain, such as set forth in SEQ ID NO:2, with the heavy chain region and the light chain region of the chimeric antibody in a host cell. In some embodiments, the IL15Rα sushi domain, such as set forth in SEQ ID NO:2, is co-expressed with the heavy chain region and the light chain region of the chimeric antibody in a host cell.

In some embodiments, the IL-15 cytokine sequence is linked to all or a portion of the IL15Rα extracellular domain, e.g., the IL15Rα sushi domain, such as set forth in SEQ ID NO:2. In some embodiments, the IL-15 sequence and the IL15Rα sushi domain sequence are placed between the ascending and descending stalk of the ultralong CDR3.

In some embodiments, the heavy chain or variable sequence thereof of the chimeric antibody is linked to the extracellular domain of the IL15Rα, such as the IL15Rα sushi domain. In some embodiments, the light chain or variable sequence thereof of the chimeric antibody is linked to the extracellular domain of the IL15Rα, such as the IL15Rα sushi domain.

In some embodiments, provided herein is a chimeric IL-15 modified antibody or antigen-binding fragment containing a heavy chain or variable sequence thereof in which an IL-15 sequence replaces all or a portion of the knob of an ultralong CDR3 (e.g., is placed between the ascending and descending stalk of the ultralong CDR3), and a light chain or variable sequence thereof that is linked to an extracellular domain of the IL15Rα, such as the IL15Rα sushi domain. In some embodiments, the chimeric IL-15 modified antibody or antigen-binding fragment is linked to an IL15Rα sushi domain set forth in SEQ ID NO:2. In some embodiments, the light chain comprises the sequence encoded by SEQ ID NO: 168 or is a variable sequence thereof. In some embodiments, the light chain comprises the sequence set forth in SEQ ID NO: 181 or is a variable sequence thereof. The linkage between the extracellular domain of the IL15Rα (e.g., the IL15Rα sushi domain, such as set forth in SEQ ID NO:2) and the light chain or variable sequence thereof is via a peptide linker. In some embodiments, the linker is a flexible linker, such as a glycine linker or a glycine-serine (GS) linker. In some embodiments, the peptide linker is a GS linker. Exemplary GS linkers include, but are not limited to, any of the sequences set forth in SEQ ID NOs: 150-154 or encoded by the nucleotide sequences set forth in SEQ ID NO:163 or SEQ ID NO:164. In some embodiments, the linker is GS.

In some embodiments, a chimeric IL-15 modified antibody or antigen-binding fragment provided herein contains a heavy chain or variable sequence thereof in which an IL-15 sequence replaces all or a portion of the knob of an ultralong CDR3 (e.g., is placed between the ascending and descending stalk of the ultralong CDR3), and a light chain or variable sequence thereof comprising the sequence of amino acids encoded by SEQ ID NO:3.

### D. Vectors, Host Cells and Recombinant Methods

The provided chimeric antibodies or antigen-binding fragments can be produced according to any suitable method, for instance those involving use of a polynucleotide encoding the antibody or fragment thereof, or the heavy chain or light chain thereof. As an example, the polynucleotide can be inserted into a replicable vector used for eventual expression of the provided chimeric antibodies or antigen-binding fragments, for instance expression by a host cell in which the vector is introduced. Such polynucleotides, vectors, e.g., expression vectors, and host cells are also provided herein and include any as described herein.

For recombinant production of an antibody or fragment thereof as disclosed herein, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). In an exemplary embodiment, a nucleic acid encoding an antibody comprising an ultralong CDR3, a variable region comprising an ultralong CDR3, or an ultralong CDR3, is isolated and inserted into a replicable vector for further cloning (amplification of the DNA) or for expression. Many vectors are available. The choice of vector depends in part on the host cell to be used. Generally, preferred host cells are of either prokaryotic or eukaryotic (generally mammalian) origin. It will be appreciated that constant regions of any isotype can be used for this purpose, including IgG, IgM, IgA, IgD, and IgE constant regions, and that such constant regions can be obtained from any human or animal species.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a partially human ultralong CDR3 antibody chain under the direction of the polyhedrin promoter or other strong baculovirus promoters.

Polynucleotide sequences encoding polypeptide components of the antibodies disclosed herein can be obtained using standard recombinant techniques. In some embodiments, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present disclosure. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to: an origin of replication, a selection marker gene, a promoter, a ribosome binding site (RBS), a signal sequence, the heterologous nucleic acid insert and a transcription termination sequence. Additionally, V regions comprising an ultralong CDR3 may optionally be fused to a C-region to produce an antibody comprising constant regions.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. pBR322 contains genes encoding ampicillin (Amp) and tetracycline (Tet) resistance and thus provides easy means for identifying transformed cells. pBR322, its derivatives, or other microbial plasmids or bacteriophage may also contain, or be modified to contain, promoters which can be used by the microbial organism for expression of endogenous proteins. Examples of pBR322 derivatives used for expression of particular antibodies have been described (see, e.g., U.S. Pat. No. 5,648,237).

In addition, phage vectors containing replicon and control sequences that are compatible with the host microorganism can be used as transforming vectors in connection with these hosts. For example, bacteriophage such as λGEM™-11 may be utilized in making a recombinant vector which can be used to transform susceptible host cells such as E. coli LE392.

The expression vectors disclosed herein may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g., the presence or absence of a nutrient or a change in temperature.

A large number of promoters recognized by a variety of potential host cells are well known. The selected promoter can be operably linked to cistron DNA encoding the light or heavy chain by removing the promoter from the source DNA via restriction enzyme digestion and inserting the isolated promoter sequence into the vector disclosed herein. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include: an ara B promoter, a PhoA promoter, β-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleotide sequences have been published, thereby enabling a skilled worker operably to ligate them to cistrons encoding the target light and heavy chains (e.g., Siebenlist et al. (1980) Cell 20:269) using linkers or adaptors to supply any required restriction sites.

Suitable bacterial promoters are well known in the art and fully described in scientific literature such as Sambrook and Russell, supra, and Ausubel et al, supra. Bacterial expression systems for expressing antibody chains of the recombinant catalytic polypeptide are available in, e.g., E. coli, Bacillus sp., and Salmonella (Palva et al., Gene, 22:229-235 (1983); Mosbach et al., Nature, 302:543-545 (1983)).

In one aspect disclosed herein, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence should be one that is recognized and processed (e.g., cleaved by a signal peptidase) by the host cell. For prokaryotic host cells that do not recognize and process the signal sequences native to the heterologous polypeptides, the signal sequence is substituted by a prokaryotic signal sequence selected, for example PelB, OmpA, alkaline phosphatase, penicillinase, Ipp, or heat-stable enterotoxin II (STII) leaders, LamB, PhoE, and MBP. In one embodiment disclosed herein, the signal sequences used in both cistrons of the expression system are STII signal sequences or variants thereof.

In another aspect, the production of the immunoglobulins according to the disclosure can occur in the cytoplasm of the host cell, and therefore does not require the presence of secretion signal sequences within each cistron. In that regard, immunoglobulin light and heavy chains are expressed, folded and assembled to form functional immunoglobulins within the cytoplasm. Certain host strains (e.g., the E. coli trxB-strains) provide cytoplasm conditions that are favorable for disulfide bond formation, thereby permitting proper folding and assembly of expressed protein subunits (see e.g., Proba and Pluckthun Gene, 159:203 (1995)).

Suitable host cells for cloning or expression of antibody-encoding vectors include prokaryotic or eukaryotic cells described herein. In one embodiment, the host cell is eukaryotic, e.g. a Chinese Hamster Ovary (CHO) cell, Human Embryonic Kidney (HEK) cell or lymphoid cell (e.g., YO, NSO, Sp20 cell). For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified. In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gemgross, Nat. Biotech. 22:1409-1414 (2004), and Li et al., Nat. Biotech. 24:210-215 (2006). Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well-known plasmids such as pBR322, pBR325, pACYC177, or pKN410 are used to supply the replicon. Typically the host cell should secrete minimal amounts of proteolytic enzymes, and additional protease inhibitors may desirably be incorporated in the cell culture.

Plant cell cultures can also be utilized as hosts. See, e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., Gen VII′01. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (V ERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells, as described, e.g., in Mather et al., Annals NI′. Acad. Sci. 383:44-68 (1982); MRC 5 cells; and FS4 cells. Other useful mammalian host cell lines include Chinese hamster ovary (CHO) cells, including DHFR′ CHO cells (Urlaub et al., Proc. Natl. Acad. Sci. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.].), pp. 255-268 (2003).

In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody.

Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

The expressed polypeptides of the present disclosure are secreted into and recovered from the periplasm of the host cells or transported into the culture media. Protein recovery from the periplasm typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins that are transported into the culture media may be isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Antibody production may be conducted in large quantity by a fermentation process. Various large-scale fed-batch fermentation procedures are available for production of recombinant proteins. Large-scale fermentations have at least 1000 liters of capacity, preferably about 1,000 to 100,000 liters of capacity. These fermentors use agitator impellers to distribute oxygen and nutrients, especially glucose (a preferred carbon/energy source). Small scale fermentation refers generally to fermentation in a fermentor that is no more than approximately 100 liters in volumetric capacity, and can range from about 1 liter to about 100 liters.

In a fermentation process, induction of protein expression is typically initiated after the cells have been grown under suitable conditions to a desired density, e.g., an OD550 of about 180-220, at which stage the cells are in the early stationary phase. A variety of inducers may be used, according to the vector construct employed, as is known in the art and described above. Cells may be grown for shorter periods prior to induction. Cells are usually induced for about 12-50 hours, although longer or shorter induction time may be used.

To improve the production yield and quality of the polypeptides disclosed herein, various fermentation conditions can be modified. For example, to improve the proper assembly and folding of the secreted antibody polypeptides, additional vectors overexpressing chaperone proteins, such as Dsb proteins (DsbA, DsbB, DsbC, DsbD and or DsbG) or FkpA (a peptidylprolyl cis, trans-isomerase with chaperone activity) may be used to co-transform the host prokaryotic cells. The chaperone proteins have been demonstrated to facilitate the proper folding and solubility of heterologous proteins produced in bacterial host cells. (see e.g., Chen et al. (1999) J Bio Chem 274:19601-19605; U.S. Pat. Nos. 6,083,715; 6,027,888; Bothmann and Pluckthun (2000) J. Biol. Chem. 275:17100-17105; Ramm and Pluckthun (2000) J. Biol. Chem. 275:17106-17113; Arie et al. (2001) Mol. Microbiol. 39:199-210).

To minimize proteolysis of expressed heterologous proteins (especially those that are proteolytically sensitive), certain host strains deficient for proteolytic enzymes can be used for the present disclosure. For example, host cell strains may be modified to effect genetic mutation(s) in the genes encoding known bacterial proteases such as Protease III, OmpT, DegP, Tsp, Protease I, Protease Mi, Protease V, Protease V1 and combinations thereof. Some E. coli protease-deficient strains are available (see, e.g., Joly et al. (1998), supra; U.S. Pat. Nos. 5,264,365; 5,508,192; Hara et al., Microbial Drug Resistance, 2:63-72 (1996)).

E. coli strains deficient for proteolytic enzymes and transformed with plasmids overexpressing one or more chaperone proteins may be used as host cells in the expression systems disclosed herein.

Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75.

In one aspect, Protein A immobilized on a solid phase is used for immunoaffinity purification of the full length antibody products disclosed herein. Protein A is a 41 kD cell wall protein from Staphylococcus aureas which binds with a high affinity to the Fc region of antibodies (see, e.g., Lindmark et al (1983) J. Immunol. Meth. 62:1-13). The solid phase to which Protein A is immobilized is preferably a column comprising a glass or silica surface, more preferably a controlled pore glass column or a silicic acid column. In some applications, the column has been coated with a reagent, such as glycerol, in an attempt to prevent nonspecific adherence of contaminants.

As the first step of purification, the preparation derived from the cell culture as described above is applied onto the Protein A immobilized solid phase to allow specific binding of the antibody of interest to Protein A. The solid phase is then washed to remove contaminants non-specifically bound to the solid phase. Finally the antibody of interest is recovered from the solid phase by elution.

### III. PHARMACEUTICAL COMPOSITIONS

Antibodies or antigen binding fragments comprising an ultralong CDR3, nucleic acids, or vectors disclosed herein can be formulated in compositions, especially pharmaceutical compositions. Such compositions with antibodies comprising an ultralong CDR3 comprise a therapeutically or prophylactically effective amount of antibodies comprising an ultralong CDR3, antibody fragment, nucleic acid, or vector disclosed herein in admixture with a suitable carrier, e.g., a pharmaceutically acceptable agent. Typically, antibodies comprising an ultralong CDR3, antibody fragments, nucleic acids, or vectors disclosed herein are sufficiently purified for administration before formulation in a pharmaceutical composition. Such pharmaceutical compositions are provided herein and include any as described herein.

Pharmaceutically acceptable agents for use in the present pharmaceutical compositions include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

Neutral buffered saline or saline mixed with serum albumin are exemplary appropriate carriers. The pharmaceutical compositions may include antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics, or polyethylene glycol (PEG). Also by way of example, suitable tonicity enhancing agents include alkali metal halides (preferably sodium or potassium chloride), mannitol, sorbitol, and the like. Suitable preservatives include benzalkonium chloride, thimerosal, phenethyl alcohol, methylparaben, propylparaben, chlorhexidine, sorbic acid and the like. Hydrogen peroxide also may be used as preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal, and the like. The buffers may be conventional buffers such as acetate, borate, citrate, phosphate, bicarbonate, or Tris-HCl. Acetate buffer may be about pH 4-5.5, and Tris buffer can be about pH 7-8.5. Additional pharmaceutical agents are set forth in Remington's Pharmaceutical Sciences, 18th Edition, A. R. Gennaro, ed., Mack Publishing Company, 1990.

The composition may be in liquid form or in a lyophilized or freeze-dried form and may include one or more lyoprotectants, excipients, surfactants, high molecular weight structural additives and/or bulking agents (see, for example, U.S. Pat. Nos. 6,685,940, 6,566,329, and 6,372,716). In one embodiment, a lyoprotectant is included, which is a non-reducing sugar such as sucrose, lactose or trehalose. The amount of lyoprotectant generally included is such that, upon reconstitution, the resulting formulation will be isotonic, although hypertonic or slightly hypotonic formulations also may be suitable. In addition, the amount of lyoprotectant should be sufficient to prevent an unacceptable amount of degradation and/or aggregation of the protein upon lyophilization. Exemplary lyoprotectant concentrations for sugars (e.g., sucrose, lactose, trehalose) in the pre-lyophilized formulation are from about 10 mM to about 400 mM. In another embodiment, a surfactant is included, such as for example, nonionic surfactants and ionic surfactants such as polysorbates (e.g., polysorbate 20, polysorbate 80); poloxamers (e.g., poloxamer 188); poly(ethylene glycol) phenyl ethers (e.g., Triton); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl ofeyl-taurate; and the MONAQUAT™. series (Mona Industries, Inc., Paterson, N.J.), polyethyl glycol, polypropyl glycol, and copolymers of ethylene and propylene glycol (e.g., Pluronics, PF68 etc). Exemplary amounts of surfactant that may be present in the pre-lyophilized formulation are from about 0.001-0.5%. High molecular weight structural additives (e.g., fillers, binders) may include for example, acacia, albumin, alginic acid, calcium phosphate (dibasic), cellulose, carboxymethylcellulose, carboxymethylcellulose sodium, hydroxyethylcellulose, hydroxypropylcellulose, hydroxypropylmethylcellulose, microcrystalline cellulose, dextran, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth microcrystalline cellulose, starch, and zein. Exemplary concentrations of high molecular weight structural additives are from 0.1% to 10% by weight. In other embodiments, a bulking agent (e.g., mannitol, glycine) may be included.

Compositions may be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically will be a sterile, pyrogen-free, isotonic aqueous solution, optionally containing pharmaceutically acceptable preservatives.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, anti-oxidants, chelating agents, inert gases and the like. See generally, Remington's Pharmaceutical Science, 16th Ed., Mack Eds., 1980.

Pharmaceutical compositions described herein may be formulated for controlled or sustained delivery in a manner that provides local concentration of the product (e.g., bolus, depot effect) and/or increased stability or half-life in a particular local environment. The compositions can include the formulation of antibodies comprising an ultralong CDR3, antibody fragments, nucleic acids, or vectors disclosed herein with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then can be delivered as a depot injection. Techniques for formulating such sustained- or controlled-delivery means are known and a variety of polymers have been developed and used for the controlled release and delivery of drugs. Such polymers are typically biodegradable and biocompatible. Polymer hydrogels, including those formed by complexation of enantiomeric polymer or polypeptide segments, and hydrogels with temperature or pH sensitive properties, may be desirable for providing drug depot effect because of the mild and aqueous conditions involved in trapping bioactive protein agents (e.g., antibodies comprising an ultralong CDR3). See, for example, the description of controlled release porous polymeric microparticles for the delivery of pharmaceutical compositions in WO 93/15722.

Suitable materials for this purpose include polylactides (see, e.g., U.S. Pat. No. 3,773,919), polymers of poly-(a-hydroxycarboxylic acids), such as poly-D-(−)-3-hydroxybutyric acid (EP 133,988A), copolymers of L-glutamic acid and gamma ethyl-L-glutamate (Sidman et al., Biopolymers, 22:547-556 (1983)), poly(2-hydroxyethyl-methacrylate) (Langer et al., J. Biomed. Mater. Res., 15:167-277 (1981), and Langer, Chem. Tech., 12:98-105 (1982)), ethylene vinyl acetate, or poly-D(−)-3-hydroxybutyric acid. Other biodegradable polymers include poly(lactones), poly(acetals), poly(orthoesters), and poly(orthocarbonates). Sustained-release compositions also may include liposomes, which can be prepared by any of several methods known in the art (see, e.g., Eppstein et al., Proc. Natl. Acad. Sci. USA, 82:3688-92 (1985)). The carrier itself, or its degradation products, should be nontoxic in the target tissue and should not further aggravate the condition. This can be determined by routine screening in animal models of the target disorder or, if such models are unavailable, in normal animals.

Microencapsulation of recombinant proteins for sustained release has been performed successfully with human growth hormone (rhGH), interferon-(rhIFN-), interleukin-2, and MN rgp120. Johnson et al., Nat. Med., 2:795-799 (1996); Yasuda, Biomed. Ther., 27:1221-1223 (1993); Hora et al., Bio/Technology. 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010. The sustained-release formulations of these proteins were developed using poly-lactic-coglycolic acid (PLGA) polymer due to its biocompatibility and wide range of biodegradable properties. The degradation products of PLGA, lactic and glycolic acids can be cleared quickly within the human body. Moreover, the degradability of this polymer can be depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41. Additional examples of sustained release compositions include, for example, EP 58,481A, U.S. Pat. No. 3,887,699, EP 158,277A, Canadian Patent No. 1176565, U. Sidman et al., Biopolymers 22, 547 [1983], R. Langer et al., Chem. Tech. 12, 98 [1982], Sinha et al., J. Control. Release 90, 261 [2003], Zhu et al., Nat. Biotechnol. 18, 24 [2000], and Dai et al., Colloids Surf B Biointerfaces 41, 117 [2005].

Bioadhesive polymers are also contemplated for use in or with compositions of the present disclosure. Bioadhesives are synthetic and naturally occurring materials able to adhere to biological substrates for extended time periods. For example, Carbopol and polycarbophil are both synthetic cross-linked derivatives of poly(acrylic acid). Bioadhesive delivery systems based on naturally occurring substances include for example hyaluronic acid, also known as hyaluronan. Hyaluronic acid is a naturally occurring mucopolysaccharide consisting of residues of D-glucuronic and N-acetyl-D-glucosamine. Hyaluronic acid is found in the extracellular tissue matrix of vertebrates, including in connective tissues, as well as in synovial fluid and in the vitreous and aqueous humor of the eye. Esterified derivatives of hyaluronic acid have been used to produce microspheres for use in delivery that are biocompatible and biodegradable (see, for example, Cortivo et al., Biomaterials (1991) 12:727-730; EP 517,565; WO 96/29998; Illum et al., J. Controlled Rel. (1994) 29:133-141). Exemplary hyaluronic acid containing compositions of the present disclosure comprise a hyaluronic acid ester polymer in an amount of approximately 0.1% to about 40% (w/w) of an antibody comprising an ultralong CDR3 to hyaluronic acid polymer.

Both biodegradable and non-biodegradable polymeric matrices may be used to deliver compositions of the present disclosure, and such polymeric matrices may comprise natural or synthetic polymers. Biodegradable matrices are preferred. The period of time over which release occurs is based on selection of the polymer. Typically, release over a period ranging from between a few hours and three to twelve months is most desirable. Exemplary synthetic polymers which may be used to form the biodegradable delivery system include: polymers of lactic acid and glycolic acid, polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyanhydrides, polyurethanes and co-polymers thereof, poly(butic acid), poly(valeric acid), alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone. Exemplary natural polymers include alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The polymer optionally is in the form of a hydrogel (see, for example, WO 04/009664, WO 05/087201, Sawhney, et al., Macromolecules, 1993, 26, 581-587) that can absorb up to about 90% of its weight in water and further, optionally is cross-linked with multi-valent ions or other polymers.

Delivery systems also include non-polymer systems that are lipids including sterols such as cholesterol, cholesterol esters and fatty acids or neutral fats such as mono-di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the product is contained in a form within a matrix such as those described in U.S. Pat. Nos. 4,452,775, 4,675,189 and 5,736,152 and (b) diffusional systems in which a product permeates at a controlled rate from a polymer such as described in U.S. Pat. Nos. 3,854,480, 5,133,974 and 5,407,686. Liposomes containing the product may be prepared by methods known methods, such as for example (DE 3,218,121; Epstein et al., Proc. Natl. Acad. Sci. USA, 82:3688-3692 (1985); Hwang et al., Proc. Natl. Acad. Sci. USA, 77:4030-4034 (1980); EP 52,322; EP 36,676; EP 88,046; EP 143,949; EP 142,641; JP 83-118008; U.S. Pat. Nos. 4,485,045 and 4,544,545; and EP 102,324).

Alternatively or additionally, the compositions may be administered locally via implantation into the affected area of a membrane, sponge, or other appropriate material on to which an antibody comprising an ultralong CDR3, antibody fragment, nucleic acid, or vector disclosed herein has been absorbed or encapsulated. Where an implantation device is used, the device may be implanted into any suitable tissue or organ, and delivery of an antibody comprising an ultralong CDR3 antibody fragment, nucleic acid, or vector disclosed herein can be directly through the device via bolus, or via continuous administration, or via catheter using continuous infusion.

A pharmaceutical composition comprising an antibody comprising an ultralong CDR3, antibody fragment, nucleic acid, or vector disclosed herein may be formulated for inhalation, such as for example, as a dry powder. Inhalation solutions also may be formulated in a liquefied propellant for aerosol delivery. In yet another formulation, solutions may be nebulized. Additional pharmaceutical composition for pulmonary administration include, those described, for example, in WO 94/20069, which discloses pulmonary delivery of chemically modified proteins. For pulmonary delivery, the particle size should be suitable for delivery to the distal lung. For example, the particle size may be from 1 μm to 5 μm; however, larger particles may be used, for example, if each particle is fairly porous.

Certain formulations containing antibodies comprising an ultralong CDR3, antibody fragments, nucleic acids, or vectors disclosed herein may be administered orally. Formulations administered in this fashion may be formulated with or without those carriers customarily used in the compounding of solid dosage forms such as tablets and capsules. For example, a capsule can be designed to release the active portion of the formulation at the point in the gastrointestinal tract when bioavailability is maximized and pre-systemic degradation is minimized. Additional agents may be included to facilitate absorption of a selective binding agent. Diluents, flavorings, low melting point waxes, vegetable oils, lubricants, suspending agents, tablet disintegrating agents, and binders also can be employed.

Another preparation may involve an effective quantity of an antibody comprising an ultralong CDR3, antibody fragment, nucleic acid, or vector disclosed herein in a mixture with non-toxic excipients which are suitable for the manufacture of tablets. By dissolving the tablets in sterile water, or another appropriate vehicle, solutions may be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Suitable and/or preferred pharmaceutical formulations may be determined in view of the present disclosure and general knowledge of formulation technology, depending upon the intended route of administration, delivery format, and desired dosage. Regardless of the manner of administration, an effective dose may be calculated according to patient body weight, body surface area, or organ size. Further refinement of the calculations for determining the appropriate dosage for treatment involving each of the formulations described herein are routinely made in the art and is within the ambit of tasks routinely performed in the art. Appropriate dosages may be ascertained through use of appropriate dose-response data.

In some embodiments, antibodies comprising an ultralong CDR3 or fragments thereof are provided with a modified Fc region where a naturally-occurring Fc region is modified to increase the half-life of the antibody or fragment in a biological environment, for example, the serum half-life or a half-life measured by an in vitro assay. Methods for altering the original form of a Fc region of an IgG also are described in U.S. Pat. No. 6,998,253.

In certain embodiments, it may be desirable to modify the antibody or fragment in order to increase its serum half-life, for example, adding molecules such as PEG or other water soluble polymers, including polysaccharide polymers, to antibody fragments to increase the half-life. This may also be achieved, for example, by incorporation of a salvage receptor binding epitope into the antibody fragment (e.g., by mutation of the appropriate region in the antibody fragment or by incorporating the epitope into a peptide tag that is then fused to the antibody fragment at either end or in the middle, e.g., by DNA or peptide synthesis) (see, International Publication No. WO96/32478). Salvage receptor binding epitope refers to an epitope of the Fc region of an IgG molecule (e.g., IgG1, IgG2, IgG3, or IgG4) that is responsible for increasing the in vivo serum half-life of the IgG molecule.

A salvage receptor binding epitope may include a region wherein any one or more amino acid residues from one or two loops of an Fc domain are transferred to an analogous position of the antibody fragment. Even more preferably, three or more residues from one or two loops of the Fc domain are transferred. Still more preferred, the epitope is taken from the CH2 domain of the z×s region (e.g., of an IgG) and transferred to the CH1, CH3, or VH region, or more than one such region, of the antibody. Alternatively, the epitope is taken from the CH2 domain of the Fc region and transferred to the CL region or VL region, or both, of the antibody fragment. See also WO 97/34631 and WO 96/32478 which describe Fc variants and their interaction with the salvage receptor.

### IV. METHODS OF USE

Provided herein are methods for using and uses of the compositions containing a chimeric cytokine (e.g., IL-15) modified antibody or antigen binding fragment, including in connection with modulation of immune cells such as T cells and NK cells and in methods for treating a disease or condition.

In some embodiments, provided herein are methods of stimulating cells, e.g., immune cells, using the chimeric antibody. In some embodiments, provided herein are methods of expanding cells, e.g., immune cells, using the chimeric antibody.

In some embodiments, a population of cells, e.g., immune cells, is contacted with the chimeric antibody, thereby stimulating cells of the population of cells, e.g., immune cells. In some embodiments, a population of cells, e.g., immune cells, is contacted with the chimeric antibody, thereby promoting proliferation of cells of the population of cells, e.g., immune cells.

In some embodiments, the population of cells, e.g., immune cells, includes cells expressing an IL-15 receptor subunit, such as an IL2/15Rβ and/or an IL2/15Rβ γc receptor subunit. In some embodiments, the population of cells, e.g., immune cells, includes T cells. In some embodiments, the population of cells, e.g., immune cells, includes natural killer (NK) cells.

In some embodiments, the provided methods are performed ex vivo or in vitro. In some embodiments, the provided methods are performed in vivo. In some embodiments, the provided methods are performed upon administration of the chimeric antibody to a subject, for instance a subject having a disease or condition.

Also provided herein are methods and uses of the chimeric antibody or compositions containing a cytokine (e.g., IL-15) modified antibody or antigen binding fragment for treating a disease or condition. In particular embodiments, the disease or condition is one that is treatable with a cytokine, and the chimeric antibody includes a cytokine sequence. In some embodiments, the disease or condition is treatable with IL-2 or IL-15 alone or in combination with another agent. In some embodiments, the chimeric antibody includes an IL-2 or an IL-25 sequence or a biologically active portion thereof. In some embodiments, the provided chimeric antibodies or antigen binding fragments are particularly suitable for use as an immunotherapy. In particular aspects, the provided chimeric antibodies or antigen-binding fragments, or compositions thereof, have use in a number of oncology applications, such as cancer, by promoting T cell activation and/or proliferation or NK cell expansion. In particular aspects, the provided chimeric antibodies or antigen-binding fragments, or compositions thereof, are modified to have reduced effector activity and avoid inducing certain effects, such as ADCC, e.g., ADCC directed against cells targeted for stimulation with the chimeric antibody, while still promoting T cell activation and/or proliferation. In some embodiments, the chimeric antibody does not induce ADCC against cells to which the chimeric antibody binds, e.g., an immune cell, for instance one expressing IL2/15Rβ and/or an IL2/15Rβ γc receptor subunits when the chimeric antibody includes an IL-15 sequence or a biologically active portion thereof. In some embodiments, the provided chimeric antibody or antigen binding fragment are used for treating cancer in a subject in need thereof.

Such methods and uses include therapeutic methods and uses, for example, involving administration of the molecules to a subject having a disease, condition or disorder, such as a cancer, to effect treatment of the disease or disorder. Uses include uses of the compositions in such methods and treatments, and uses of such compositions in the preparation of a medicament in order to carry out such therapeutic methods. In some embodiments, the methods and uses thereby treat the disease or condition or disorder, such as a tumor or cancer, in the subject.

In some embodiments, the cancer is a blood cancer, such as a lymphoma, leukemia or myeloma. In some embodiments, the cancer is a solid tumor cancer. In some embodiments, the cancer is a cancer of the head and neck, breast, liver, colon, ovary, prostate, pancreas, brain, cervix, bone, skin, lung, or blood. In some embodiments, cancer may include a malignant tumor characterized by abnormal or uncontrolled cell growth. Other features that may be associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. Metastatic disease may refer to cancer cells that have left the original tumor site and migrated to other parts of the body, for example via the bloodstream or lymph system.

In some embodiments, the provided methods result in an amelioration of and or treat the disease or condition, such as cancer. In some aspects, the provided methods result in one or more improvements in the disease, such as a reduction in the number of neoplastic cells, an increase in neoplastic cell death, inhibition of neoplastic cell survival, inhibition (i.e. slowing to some extent or halting) of tumor growth, an increase in patient survival rate, and/or some relief from one or more symptoms associated with the disease or condition.

In aspects of the provided methods, response can be assessed or determined using criteria specific to the disease or condition. In some embodiments, tumor response can be assessed for changes in tumor morphology (i.e. overall tumor burden, tumor size) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

The provided methods involve administering a therapeutically effective amount of the compositions provided herein to a subject in need thereof, such as a cancer subject. A therapeutically effective amount may vary according to factors such as the disease state age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects. In some cases, a therapeutically effective amount for tumor or cancer therapy may also be measured by its ability to stabilize the progression of disease. The ability of the provided antibody or antigen binding fragments to inhibit cancer may be evaluated in an animal model system predictive of efficacy in human tumors.

Alternatively, this property of a composition may be evaluated by examining the ability of the antibody or antigen binding fragment to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

In some embodiments, the provided antibodies or antigen binding fragments can be administered in a single dose, or in several doses, as needed to obtain the desired response. In some embodiments, the effective amount is dependent on the source applied, the subject being treated, the severity and type of the condition being treated, and the manner of administration.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound cal-culated to produce the desired therapeutic effect in associa-tion with the required pharmaceutical carrier.

In some embodiments, the therapeutically effective amount is between at or about 0.1 to 100 mg/kg, or any value between any of the foregoing.

In some embodiments, the provided methods and uses can be carried out in combination with another therapy, such as another therapy for treating the disease or condition. In some embodiments, the disease or condition is a tumor or cancer and the other therapy is an anti-tumor agent or therapy (also referred to herein as anti-cancer agent or therapy). In particular, the provided methods can be used in connection with cancer immunotherapy. Cancer immunotherapy aims to eradicate cancer cells by rejuvenating the tumoricidal functions of tumor-reactive immune cells, such as T cells or NK cells. Strategies of cancer immunotherapy including checkpoint blockade, adoptive cell transfer (ACT) and cancer vaccines which can increase the anti-tumor immune effector cells have produced remarkable results in several tumors. In some embodiments, the anti-tumor or anti-cancer therapy is an antibody therapeutic, such as a monoclonal antibody.

In some aspects, anti-tumor agents suitable for use in the provided methods and uses include those that promote ADCC against tumor cells. For instance, in some aspects, the chimeric antibody is modified to reduce effector function and does not interfere or compete with the ability of the anti-tumor agent to promote ADCC against tumor cells, for instance via the anti-tumor agent's engagement with immune cells, e.g., via FcR binding. In some embodiments, for instance when the anti-tumor agent includes a cell therapy, the chimeric antibody does not bind to or has reduced binding to FcRs expressed by the cell therapy, and the chimeric antibody does not induce ADCC of the anti-tumor agent against cells to which the chimeric antibody binds. Anti-tumor agents that do not promote ADCC are also suitable for use in the methods and uses provided herein.

A problem with certain cancer immunotherapy approaches is that host anti-tumor immunity can impede the efficacy of cancer immunotherapy. For instance, in some cases, formation of an immunosuppressive tumor microenvironment may impact the ability of natural tumor-reactive immune cells or adoptively transferred immune cells from successfully eradicating cancer cells. Particularly, in solid tumors the therapeutic efficacy of immunotherapeutic regimens remains unsatisfactory due to lack of an effective an anti-tumor response in the immunosuppressive tumor microenvironment. Tumor cells often induce immune tolerance or suppression and such tolerance is acquired because even truly foreign tumor antigens will become tolerated. Such tolerance is also active and dominant because cancer vaccines and adoptive transfer of pre-activated immune effector cells (e.g., T cells), are subject to suppression by inhibitory factors in the tumor microenvironment (TME).

In some embodiments, the chimeric cytokine (e.g. IL-15) modified antibody or antigen-binding fragment is administered in combination with checkpoint blockade agents (also called an immune checkpoint inhibitor). An immune checkpoint inhibitor is a molecule that totally or partially reduces, inhibits, interferes with or modulates one or more checkpoint proteins. Checkpoint proteins regulate T-cell activation or function. These proteins are responsible for co-stimulatory or inhibitory interactions of T-cell responses. Immune checkpoint proteins regulate and maintain self-tolerance and the duration and amplitude of physiological immune responses.

Immune checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptor ligands. Illustrative immune checkpoint molecules that may be targeted for blocking or inhibition include, but are not limited to, PD1 (CD279), PDL1 (CD274, B7-H1), PDL2 (CD273, B7-DC), CTLA-4, LAG3 (CD223), TIM3, 4-1BB (CD137), 4-1BBL (CD137L), GITR (TNFRSF18, AITR), CD40, Ox40 (CD134, TNFRSF4), CXCR2, tumor associated antigens (TAA), B7-H3, B7-H4, BTLA, HVEM, GAL9, B7H3, B7H4, VISTA, KIR, 2B4 (belongs to the CD2 family of molecules and is expressed on all NK, γδ, and memory CD8+ (αβ) T cells), CD160 (also referred to as BY55) and CGEN-15049. Immune checkpoint inhibitors include antibodies, or antigen binding fragments thereof, or other binding proteins, that bind to and block or inhibit the activity of one or more of PD1, PDL1, PDL2, CTLA-4, LAG3, TIM3, 4-1BB, 4-1BBL, GITR, CD40, Ox40, CXCR2, TAA, B7-H3, B7-H4, BTLA, HVEM, GAL9, B7H3, B7H4, VISTA, KIR, 2B4, CD160, and CGEN-15049. Illustrative immune checkpoint inhibitors include Tremelimumab (CTLA-4 blocking antibody), anti-OX40, PD-L1 monoclonal antibody (Anti-B7-H1; MEDI4736), MK-3475 (PD-1 blocker), nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor).

In some embodiments, the immune checkpoint inhibitor specifically binds a molecule selected from among CD25, PD-1, PD-L1, PD-L2, CTLA-4, LAG-3, TIM-3, 4-1BB, GITR, CD40, CD40L, OX40, OX40L, CXCR2, B7-H3, B7-H4, BTLA, HVEM, CD28 and VISTA. In some embodiments, the immune checkpoint inhibitor is and antibody or antigen-binding fragment, a small molecule or a polypeptide. In some embodiments, the immune checkpoint inhibitor is selected from among nivolumab, pembrolizumab, pidilizumab, MK-3475, BMS-936559, MPDL3280A, ipilimumab, tremelimumab, IMP31, BMS-986016, urelumab, TRX518, dacetuzumab, lucatumumab, SEQ-CD40, CP-870, CP-893, MED16469, MEDI4736, MOXR0916, AMP-224, and MSB001078C, or is an antigen-binding fragment thereof. In some embodiments, the immune checkpoint inhibitor can be an anti-PD-1 or anti-PD-L1 antibody. Antibodies targeting PD-1 or PD-L1 include, but are not limited to, Nivolumab, Pembrolizumab or Atezolizumab.

In some embodiments, the anti-tumor agent includes a monoclonal antibody. In some embodiments, the monoclonal antibody is any as described in Zahavi et al. (2020), Antibodies (Basel) 9(3): 34. In some embodiments, the monoclonal antibody is Atezolizumab, Avelumab, Bevacizumab, Cemiplimab, Cetuximab, Daratumumab, Dinutuximab, Durvalumab, Elotuzumab, Ipilimumab, Isatuximab, Mogamulizumab, Necitumumab, Nivolumab, Obinutuzumab, Ofatumumab, Olaratumab, Panitumumab, Pembrolizumab, Pertuzumab, Ramucirumab, Rituximab, or Trastuzumab.

In some embodiments, the chimeric cytokine (e.g., IL-15) modified antibody or antigen-binding fragment is administered in combination with cell therapies such as adoptive cell therapy. In some embodiments, administration of a provided chimeric cytokine (e.g., IL-15) modified antibody or antigen-binding fragment combination with such an adoptive cell therapy may be used for stimulating T cells, such as in TCR/CAR combinations, in the manipulation or regulation of TILs, for increasing expansion of NK cells, including engineered NK cells (e.g. CAR-engineered NK cells). In some embodiments, the adoptive cell therapy may be an autologous cell therapy or may be an allogeneic cell therapy. In some embodiments, immune cells for adoptive cell therapy in provided combinations may be dendritic cells, T cells such as CD8+ T cells and CD4+ T cells, natural killer (NK) cells, NK T cells, Cytotoxic T lymphocytes (CTLs), tumor infiltrating lymphocytes (TILs), lymphokine activated killer (LAK) cells, memory T cells, regulatory T cells (Tregs), helper T cells, cytokine-induced killer (CIK) cells, and any combination thereof. In other embodiments, immune stimulatory cells for adoptive cell therapy may be generated from embryonic stem cell (ESC) and induced pluripotent stem cell (iPSC). In some embodiments, autologous or allogeneic immune cells are used for adoptive cell therapy.

In some embodiments, administration of the chimeric cytokine (e.g., IL-15) modified antibody or antigen-binding fragment, whether as a single agent or as a combination agent, results in the proliferation of immune cells. In some embodiments, administration of the chimeric cytokine modified antibody or antigen-binding fragment results in a 1.1, 1.2, 1.3, 1.4, 1.5, 1.6, 1.7, 1.8, 1.9, 2, 2.2, 2.4, 2.6, 2.8, 3, 4, or 5 fold increase in the number of immune cells. In some embodiments, the immune cells that proliferate are T cells. In some embodiments, the immune cells that proliferate are NK cells. In some embodiments, the immune cells that proliferate are T cells and NK cells.

In some embodiments, the immune cells that proliferate are immune cells that have been administered as part of a cell therapy, including any of the cell therapies described herein, and for instance a cell therapy administered in combination with the chimeric cytokine modified antibody or antigen-binding fragment. In some embodiments, the cell therapy is a T cell therapy. In some embodiments, the cell therapy is an NK cell therapy.

In some embodiments, the chimeric cytokine (e.g., IL-15) modified antibody or antigen-binding fragment is administered in combination with other agents effective in the treatment of cancers, infection diseases and other immunodeficient disorders, such as anti-cancer agents. The anti-cancer agent may be any agent which is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

In some embodiments, anti-cancer agent or therapy may be a chemotherapeutic agent, or radiotherapy, immunotherapeutic agent, surgery, or any other therapeutic agent which, in combination with the chimeric cytokine (e.g., IL-15) modified antibody or antigen-binding fragment improves the therapeutic efficacy of treatment.

In one embodiment, the chimeric cytokine (e.g., IL-15) modified antibody or antigen-binding fragment may be used in combination with amino pyrimidine derivatives such as the Burkitt's tyrosine receptor kinase (BTK) inhibitor, such as using methods taught in International Patent Application NO. WO2016164580, the contents of which are incorporated herein by reference in their entirety.

In some embodiments, the chimeric cytokine (e.g., IL-15) modified antibody or antigen-binding fragment may be used in combination with antibodies specific to some target molecules on the surface of a tumor cell.

Exemplary anti-cancer agents include, without limitation, Acivicin; Aclarubicin; Acodazole hydrochloride; Acronine; Adozelesin; Aldesleukin; Altretamine; Ambomycin; Ametantrone acetate; Amsacrine; Anastrozole; Anthramycin; Asparaginase; Asperrin, Sulindac, Curcumin, alkylating agents including: Nitrogen mustards such as mechlor-ethamine, cyclophosphamide, ifosfamide, melphalan and chlorambucil; nitrosoureas such as carmustine (BC U), lomustine (CCNU), and semustine (methyl-CC U); thylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrrolidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6˜thioguanine, azathioprine, 2,′-deoxycoformycin (pentostatin), erythrohyckoxynonyladenine (EHNA), ffudarabine phosphate, and 2-chlorodeoxyadenosine (cladribine, 2-CdA); natural products including antimitotic drugs such as paclitaxei, vinca alkaloids including vinblastine (VLB), vincristine, and vinorelbine, taxotere, estramustine, and estramustine phosphate; epipodophylotoxins such as etoposide and teniposide; antibiotics, such as actimomycin D, daunomycin (ruhidomycin), doxorubicin, mitoxantrone, idarubicin, bleomycins, plicamycm (mithramycin), mitomycinC, and actinomycin; enzymes such as -[.-asparaginase, cytokines such as interferon (IFN)-gamma, tumor necrosis factor (TNF)-alpha, TNF-beta and GM-CSF, anti-angiogenic factors, such as angiostatin and endostatin, inhibitors of FGF or VEGF such as soluble forms of receptors for angiogenic factors, including soluble VGF/VEGF receptors, platinum coordination complexes such as cisplatin and carboplatin, anthracenediones such as mitoxantrone, substituted urea such as hydroxyurea, methylhydrazme derivatives including N-methylhydrazme (MIFf) and procarbazine, adrenocortical suppressants such as mitotane (o.ρ′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fiuoxymesterone/equivalents; antiandrogens such as flutamide, gonadotropin-releasing hormone analogs and leuprolide; non-steroidal antiandrogens such as flutamide; kinase inhibitors, histone deacetylase inhibitors, methylation inhibitors, proteasome inhibitors, monoclonal antibodies, oxidants, anti-oxidants, telomerase inhibitors, BH3 mimetics, ubiquitin ligase inhibitors, stat inhibitors and receptor tyrosin kinase inhibitors such as imatinib mesylate (marketed as Gleevac or Giivac) and erlotinib (an EGF receptor inhibitor) now marketed as Tarveca; anti-virals such as oseltamivir phosphate. Amphotericin B, and palivizumab; Sdi 1 mimetics; Semusiine; Senescence derived inhibitor 1; Sparfosic acid: Spicamycin D; Spiromustine; Spienopentin: Spongistatin 1: Squaiamine: Stipiamide; Stromelysin inhibitors; Sulfinosine; Superactive vasoactive intestinal peptide antagonist; Velaresol; Veramine; Verdins; Verteporfin; Vinorelbine; Vmxaltme; Vitaxin; Vorozole; Zanoterone; Zeniplatin; Zilascorb; and Zinostatin stimalamer; PO β small-molecule inhibitor, GSK2636771; pan-PI3 inhibitor (BKM120); BRAF inhibitors. Veniurafenib (Zeiboraf) and dabrafenib (Tafmiar); or any analog or derivative and variant of the foregoing.

In some embodiments, the anti-cancer agent is an antibody or antigen-binding antibody fragment thereof that includes, but is not limited to, Daclizumab (Zenapax), Bevacizumab (Avastin®), Basiliximab, Ipilimumab, Nivolumab, pembrolizumab, MPDL3280A, Pidilizumab (CT-011), MK-3475, BMS-936559, MPDL3280A (Atezolizumab), tremelimumab, IMP321, BMS-986016, LAG525, urelumab, PF-05082566, TRX518, MK-4166, dacetuzumab (SGN-40), lucatumumab (HCD122), SEA-CD40, CP-870, CP-893, MEDI6469, MEDI6383, MOXR0916, AMP-224, MSB0010718C (Avelumab), MEDI4736, PDR001, rHIgM12B7, Ulocuplumab, BKT140, Varlilumab (CDX-1127), ARGX-110, MGA271, lirilumab (BMS-986015, IPH2101), IPH2201, ARGX-115, Emactuzumab, CC-90002 and MNRP1685A or an antibody-binding fragment thereof.

Other agents may be used in combination with the chimeric cytokine (e.g. IL-15) modified antibody or antigen-binding fragment may also include, but not limited to, agents that affect the upregulation of cell surface receptors and their ligands such as Fas/Fas ligand, DR4 or DR5/TRAIL and GAP junctions, cytostatic and differentiation agents, inhibitors of cell adhesion such as focal adhesion kinase (FAKs) inhibitors and Lovastatin, or agents that increase the sensitivity of the hyper proliferative cells to apoptotic inducers such as the antibody C225.

In some embodiments, the chimeric cytokine (e.g., IL-15) modified antibody or antigen-binding fragment is administered in combination with a cancer vaccine. In some embodiments, cancer vaccine may comprise peptides and/or proteins derived from tumor associated antigen (TAA). Such strategies may be utilized to evoke an immune response in a subject, which in some instances may be a cytotoxic T lymphocyte (CTL) response. Peptides used for cancer vaccines may also be modified to match the mutation profile of a subject. For example, EGFR derived peptides with mutations matched to the mutations found in the subject in need of therapy have been successfully used in patients with lung cancer (Li F et al. (2016) Oncoimmunology. October 7; 5(12): el 238539; the contents of which are incorporated herein by reference in their entirety). In one embodiment, cancer vaccines include a superagonist altered peptide ligands (APL) derived from TAAs. These are mutant peptide ligands deviate from the native peptide sequence by one or more amino acids, which activate specific CTL clones more effectively than native epitopes. These alterations may allow the peptide to bind better to the restricting Class I MHC molecule or interact more favorably with the TCR of a given tumor-specific CTL subset. APLs may be selected using methods taught in US Patent Publication NO. US20160317633 A 1, the contents of which are incorporated herein by reference in their entirety.

In some embodiments, the combinations may include administering the chimeric cytokine (e.g., IL-15) modified antibody or antigen-binding fragment and other agents at the same time or separately. Alternatively, the chimeric cytokine (e.g., IL-15) modified antibody or antigen-binding fragment may precede or follow the other agent/therapy by intervals ranging from minutes, days, weeks to months.

### V. EXEMPLARY EMBODIMENTS

Among the provided embodiments are:
1. A chimeric modified antibody, comprising a heavy chain comprising:
(a) a modified variable heavy (VH) region of a bovine antibody or antigen-binding fragment or a humanized sequence thereof, wherein the modified VH region comprises a modified ultralong CDR3 wherein at least a portion of an ultralong CDR3 of the bovine antibody or antigen-binding fragment or a humanized sequence thereof is replaced by a cytokine sequence or a biologically active portion thereof; and
(b) a modified human IgG heavy chain constant region with reduced effector activity compared to a wild-type human IgG heavy chain constant region.
2. The chimeric modified antibody of embodiment 1, wherein the cytokine sequence or biologically active portion thereof replaces a knob region of the ultralong CDR3 region of the bovine antibody or antigen-binding fragment or the humanized sequence thereof.
3. The chimeric modified antibody of embodiment 1 or embodiment 2, wherein the cytokine sequence or biologically active portion thereof is between an ascending stalk strand and a descending stalk strand of the modified ultralong CDR3, wherein the ascending stalk strand of the modified ultralong CDR3 is a variant compared to an ascending stalk strand of the ultralong CDR3 of the bovine antibody or antigen-binding fragment or a humanized sequence thereof.
4. The chimeric modified antibody of embodiment 3, wherein the cytokine sequence or biologically active portion thereof is linked to the ascending stalk strand and/or to the descending stalk strand of the modified ultralong CDR3 via a flexible linker, optionally a GGS or GSG linker.
5. The chimeric modified antibody of embodiment 3 or embodiment 4, wherein the ascending stalk strand comprises the sequence CX₂TVX₅QETKKYQT, wherein X₂ and X₅ are any amino acid.
6. A chimeric modified antibody, comprising a heavy chain comprising a modified variable heavy (VH) region of a bovine antibody or antigen-binding fragment or a humanized sequence thereof, wherein the modified VH region comprises a modified ultralong CDR3 in which at least a portion of an ultralong CDR3 region of the bovine antibody or antigen-binding fragment or a humanized sequence thereof is replaced by a heterologous sequence, wherein the heterologous sequence is between an ascending stalk strand and a descending stalk strand of the modified ultralong CDR3, wherein the ascending stalk strand of the modified ultralong CDR3 comprises the sequence CX₂TVX₅QETKKYQT, wherein X₂ and X₅ are any amino acid.
7. The chimeric modified antibody of embodiment 5 or embodiment 6, wherein X₂ is Ser, Thr, Gly, Asn, Ala, or Pro, and X₅ is His, Gln, Arg, Lys, Gly, Thr, Tyr, Phe, Trp, Met, Ile, Val, or Leu.
8. The chimeric modified antibody of any of embodiments 5-7, wherein X₂ is Ser, Ala, or Thr, and X₅ is His or Tyr.
9. The chimeric modified antibody of any of embodiments 3-8, wherein the ascending stalk strand of the modified ultralong CDR3 comprises the sequence set forth in any of SEQ ID NOs: 183-185.
10. The chimeric modified antibody of any of embodiments 3-8, wherein the sequence of the ascending stalk strand of the modified ultralong CDR3 is set forth in any of SEQ ID NOs: 183-185.
11. The chimeric modified antibody of any of embodiments 6-10, wherein the heterologous sequence replaces a knob region of the ultralong CDR3 region of the bovine antibody or antigen-binding fragment or the humanized sequence thereof.
12. The chimeric modified antibody of any of embodiments 6-11, wherein the heterologous sequence is linked to the ascending stalk strand and/or to the descending stalk strand of the modified ultralong CDR3 via a flexible linker, optionally a GGS or GSG linker.
13. The chimeric modified antibody of any of embodiments 6-12, wherein the heterologous sequence comprises a cytokine sequence or a biologically active portion thereof.
14. The chimeric modified antibody of any of embodiments 6-13, wherein the heavy chain further comprises a human IgG heavy chain constant region.
15. The chimeric modified antibody of embodiment 14, wherein the human IgG heavy chain constant region is a modified human IgG heavy chain constant region with reduced effector activity compared to a wild-type human IgG heavy chain constant region.
16. The chimeric modified antibody of any of embodiments 1-5 and 7-15, wherein the human IgG is human IgG1.
17. The chimeric modified antibody of any of embodiments 1-5 and 7-16, wherein the modified human IgG heavy chain constant region is modified to reduce FcR binding.
18. The chimeric modified antibody of any of embodiments 1-5 and 7-17, wherein the reduced effector activity comprises reduced antibody-dependent cell-mediated cytotoxicity (ADCC).
19. The chimeric modified antibody of any of embodiments 1-5 and 7-18, wherein the modified human IgG heavy chain constant region is altered at one or more of positions Glu233 (E233), Leu 234 (L234), Leu235 (L235), Asp265 (D265), Asp270 (D270), Asn297 (N297), Ser298 (S298), Asn325 (N325), Ala327 (A327), and Pro329 (P329).
20. The chimeric modified antibody of any of embodiments 1-5 and 7-19, wherein the modified human IgG heavy chain constant region comprises one or more mutations selected from Leu234Ala (L234A), Leu235Ala (L235A), Leu235Glu (L235E), Asp265Asn (D265N), Asp265Ala (D265A), Asp270Asn (D270N), Ser298Asn (S298N), Asn325Glu (N325E), Ala327Ser (A327S), Pro329Ala (P329A), and Pro239Gly (P329G).
21. The chimeric modified antibody of any of embodiments 1-5 and 7-20, wherein the modified human IgG heavy chain constant region is altered at two or more of positions Glu233 (E233), Leu 234 (L234), Leu235 (L235), Asp265 (D265), Asp270 (D270), Asn297 (N297), Ser298 (S298), Asn325 (N325), Ala327 (A327), and Pro329 (P329).
22. The chimeric modified antibody of any of embodiments 1-5 and 7-21, wherein the modified human IgG heavy chain constant region comprises Leu234Ala and Leu235Ala (L234A/L235A) mutations; Leu234Val and Leu235Ala (L234V/L235A) mutations; Leu234Ala, Leu235Ala, and Asn297Ala (L234A/L235A/N297A) mutations; Leu234Ala, Leu235Ala, and Pro239Ala (L234A/L235A/P329A) mutations; Asp265Ala and Pro329Ala (D265A/P329A) mutations; Asp265Ala and Pro329Gly (D265A/P329G) mutations; Leu234Ala, Leu235Ala, and Asp265Ala (L234A/L235A/D265A) mutations; Leu234Ala, Leu235Ala, and Pro329Gly (L234A/L235A/P329G) mutations; or Leu234Ala, Leu235Ala, Asp265Ala, and Pro329Gly (L234A/L235A/D265A/P329G) mutations.
23. The chimeric modified antibody of any of embodiments 1-5 and 7-22, wherein the modified human IgG heavy chain constant region comprises Leu234Ala and Leu235Ala (L234A/L235A) mutations.
24. The chimeric modified antibody of 1-5 and 7-23, wherein the modified human IgG heavy chain constant region comprises the sequence set forth in SEQ ID NO: 187 or SEQ ID NO: 188.
25. The chimeric modified antibody of any of embodiments 1-5 and 7-24, wherein the cytokine sequence or biologically active portion thereof comprises an interleukin-15 (IL-15) cytokine sequence or a biologically active portion thereof.
26. The chimeric modified antibody of any of embodiments 1-5 and 7-25, wherein the cytokine sequence or biologically active portion thereof comprises a sequence of amino acids that exhibits at least at or about 85%, at least at or about 90%, at least at or about 92%, at least at or about 95%, at least at or about 96%, at least at or about 97%, at least at or about 98%, or at least at or about 99% sequence identity to SEQ ID NO: 1.
27. The chimeric modified antibody of any of embodiments 1-5 and 7-26, wherein the cytokine sequence or biologically active portion thereof comprises the sequence set forth in SEQ ID NO: 1.
28. The chimeric modified antibody of any of embodiments 1-5 and 7-24, wherein the cytokine sequence or biologically active portion thereof comprises an interleukin-12 (IL-2) cytokine sequence or a biologically active portion thereof.
29. The chimeric modified antibody of any of embodiments 1-5, 7-24, and 28, wherein the cytokine sequence or biologically active portion thereof comprises a sequence of amino acids that exhibits at least at or about 85%, at least at or about 90%, at least at or about 92%, at least at or about 95%, at least at or about 96%, at least at or about 97%, at least at or about 98%, or at least at or about 99% sequence identity to SEQ ID NO: 165.
30. The chimeric modified antibody of any of embodiments 1-5, 7-24, 28, and 29, wherein the cytokine sequence or biologically active portion thereof comprises the sequence of amino acids set forth in SEQ ID NO: 165.
31. The chimeric modified antibody of any of embodiments 1-30, wherein the bovine antibody or antigen-binding fragment is the bovine antibody BLV1H12 or an antigen-binding fragment thereof.
32. The chimeric modified antibody of any of embodiments 3-31, wherein the descending stalk strand comprises the sequence set forth in SEQ ID NO: 10.
33. The chimeric modified antibody of any of embodiments 3-5, 7-27, 31, and 32, wherein:
the ascending stalk strand comprises the sequence set forth in SEQ ID NO: 183, the cytokine sequence or biologically active portion thereof comprises the sequence of amino acids set forth in SEQ ID NO: 1, and the descending stalk strand comprises the sequence set forth in SEQ ID NO: 10;
the ascending stalk strand comprises the sequence set forth in SEQ ID NO: 184, the cytokine sequence or biologically active portion thereof comprises the sequence of amino acids set forth in SEQ ID NO: 1, and the descending stalk strand comprises the sequence set forth in SEQ ID NO: 10; or
the ascending stalk strand comprises the sequence set forth in SEQ ID NO: 185, the cytokine sequence or biologically active portion thereof comprises the sequence of amino acids set forth in SEQ ID NO: 1, and the descending stalk strand comprises the sequence set forth in SEQ ID NO: 10.
34. The chimeric modified antibody of any of embodiments 1-27 and 31-33, wherein the modified ultralong CDR3 comprises the sequence set forth in any of SEQ ID NOs: 206-208.
35. The chimeric modified antibody of any of embodiments 1-34, wherein the modified VH region is a variant of the VH region of BLV1H12.
36. The chimeric modified antibody of any of embodiments 1-35, wherein the heavy chain comprises the formula V1-X-V2-C, wherein the V1 region of the heavy chain comprises the sequence set forth in SEQ ID NO: 182; the X region comprises the modified ultralong CDR3; the V2 region comprises the sequence set forth in SEQ ID NO: 11; and the C region comprises the modified human IgG heavy chain constant region.
37. The chimeric modified antibody of any of embodiments 1-27 and 31-36, wherein the modified VH region comprises the sequence set forth in any of SEQ ID NOs: 200-202.
38. The chimeric modified antibody of any of embodiments 1-27 and 31-37, wherein the heavy chain comprises the sequence set forth in any of SEQ ID NOs: 189-191.
39. The chimeric modified antibody of any of embodiments 1-34, wherein the modified VH region is a variant of a humanized sequence of the VH region of BLV1H12.
40. The chimeric modified antibody of any of embodiments 1-34 and 39, wherein the heavy chain comprises the formula V1-X-V2-C, wherein the V1 region of the heavy chain comprises the sequence set forth in SEQ ID NO: 197 or a sequence that exhibits at least 65% sequence identity to SEQ ID NO: 197; the X region comprises the modified ultralong CDR3; the V2 region comprises the sequence set forth in SEQ ID NO: 11; and the C region comprises the modified human IgG heavy chain constant region.
41. The chimeric modified antibody of any of embodiments 1-34, 39, and 40, wherein the heavy chain comprises the formula V1-X-V2-C, wherein the V1 region of the heavy chain comprises the sequence set forth in SEQ ID NO: 197; the X region comprises the modified ultralong CDR3; the V2 region comprises the sequence set forth in SEQ ID NO: 11; and the C region comprises the modified human IgG heavy chain constant region.
42. The chimeric modified antibody of any of embodiments 1-27, 31-34, and 39-41, wherein the modified VH region comprises the sequence set forth in any of SEQ ID NOs: 203-205.
43. The chimeric modified antibody of any of embodiments 1-27, 31-34, and 39-42, wherein the heavy chain comprises the sequence set forth in any of SEQ ID NOs: 192-194.
44. The chimeric modified antibody of any of embodiments 1-43, further comprising a light chain.
45. The chimeric modified antibody of any of embodiments 1-44, wherein the chimeric modified antibody comprises a humanized light chain.
46. The chimeric modified antibody of embodiment 45, wherein the humanized light chain comprises the sequence set forth in SEQ ID NO: 181 or a sequence that exhibits at least 85% sequence identity to SEQ ID NO:181.
47. The chimeric modified antibody of embodiment 45, wherein the humanized light chain comprises the sequence set forth in SEQ ID NO: 181.
48. The chimeric modified antibody of any of embodiments 1-27 and 31-47, wherein the chimeric modified antibody is complexed with an extracellular domain of the IL15Rα comprising the IL15Rα sushi domain.
49. The chimeric modified antibody of embodiment 48, wherein the extracellular domain of the IL15Rα comprising the IL15Rα sushi domain is non-covalently associated with the IL-15 sequence.
50. The chimeric modified antibody of embodiment 48, wherein the extracellular domain of the IL15Rα comprising the IL15Rα sushi domain is linked to the light chain of the chimeric modified antibody, optionally linked via a peptide linker.
51. The chimeric modified antibody of any of embodiments 48-50, wherein the extracellular domain of the IL15Rα comprising the IL15Rα sushi domain comprises the sequence set forth in SEQ ID NO:2.
52. A polynucleotide encoding the chimeric modified antibody of any of embodiments 1-51.
53. A polynucleotide encoding a heavy chain or a variable region thereof of the chimeric modified antibody of any of embodiments 1-51.
54. A polynucleotide encoding a light chain or a variable region thereof of the chimeric modified antibody of any of embodiments 1-51.
55. An expression vector comprising the polynucleotide of any of embodiments 52-54.
56. A host cell comprising the polynucleotide of any of embodiments 52-54 or the expression vector of embodiment 55.
57. The host cell of embodiment 56, further comprising a polynucleotide or vector encoding an extracellular domain of the IL15Rα comprising the IL15Rα sushi domain.
58. The host cell of embodiment 57, wherein the extracellular domain of the IL15Rα comprising the IL15Rα sushi domain comprises the sequence set forth in SEQ ID NO:2.
59. A method of producing a chimeric modified antibody, comprising culturing the host cell of any of embodiments 56-58 under conditions for expression of the chimeric modified antibody by the host cell, optionally further comprising recovering or purifying the chimeric modified antibody.
60. A chimeric modified antibody produced by the method of embodiment 59.
61. A pharmaceutical composition comprising the chimeric modified antibody of any of embodiments 1-51 and 60.
62. A method of stimulating immune cells, comprising contacting a population of immune cells with the chimeric modified antibody of any of embodiments 1-51 and 60, thereby stimulating cells of the population of immune cells.
63. A method of expanding immune cells, comprising contacting a population of immune cells with the chimeric modified antibody of any of embodiments 1-51 and 60, thereby promoting proliferation of cells of the population of immune cells.
64. The method of embodiment 62 or embodiment 63, wherein the population of immune cells comprises cells expressing an IL2/15Rβ and/or an IL2/15Rβ γc receptor subunit.
65. The method of any of embodiments 62-64, wherein the population of immune cells comprises T cells or natural killer (NK) cells.
66. The method of any of embodiments 62-65, wherein the method is performed ex vivo or in vitro.
67. The method of any of embodiments 62-65, wherein the method is performed in vivo upon administration of the chimeric modified antibody to a subject.
68. A method of treating a cancer in a subject, comprising administering to a subject a therapeutically effective amount of the chimeric modified antibody of any of embodiments 1-51 and 60.
69. A method of treating a cancer in a subject, comprising administering to a subject the pharmaceutical composition of embodiment 61.
70. The method of any of embodiments 67-69, further comprising administering to the subject an anti-tumor agent.
71. The method of embodiment 70, wherein the anti-tumor agent comprises a monoclonal antibody.
72. The method of embodiment 70 or embodiment 71, wherein the anti-tumor agent comprises a checkpoint inhibitor.
73. The method of any of embodiments 70-72, wherein the anti-tumor agent comprises a cell therapy, optionally a T cell therapy or an NK cell therapy.
74. The method of embodiment 73, wherein the cell therapy comprises cells expressing a chimeric antigen receptor (CAR).

### VI. EXAMPLES

The following examples are included for illustrative purposes only and are not intended to limit the scope of the invention.

### Example 1 Generation of Chimeric Interleukin (IL)-15 Fusion Antibodies

Exemplary chimeric BLV1H12-IL-15 (B15) fusion antibodies comprising an IL-15 sequence were generated by modifying the ultralong CDR3 of the bovine antibody BLV1H12 or a humanized variant thereof.

The heavy chain of BLV1H12 includes a sequence with formula V1-X-V2-C, wherein the V1 region of the heavy chain comprises a heavy chain sequence portion containing three framework regions (e.g., FR-1, FR-2, and FR-3) separating two CDR regions (CDR1 and CDR2); the X region comprises the ultralong CDR3 sequence, which includes a knob region between an ascending stalk strand and a descending stalk strand; the V2 region comprises a portion of the heavy chain including FR-4; and the C region is the constant heavy chain region. The VH regions of the B15 antibodies were engineered by replacing the knob region (SEQ ID NO: 195) of the ultralong CDR3 with an N-terminal GGS linker (SEQ ID NO: 151), an IL-15 sequence (SEQ ID NO: 1), and a C-terminal GSG linker (SEQ ID NO: 186). The VH regions were further engineered by modifying the ascending stalk strand of the ultralong CDR3 (unmodified sequence set forth in SEQ ID NO: 9). Besides the engineered VH regions, the heavy chains of the B15 antibodies also included an unmodified or a modified heavy chain constant region of human IgG1 (unmodified sequence set forth in SEQ ID NO: 196), wherein the modified constant region included Leu234Ala and Leu235Ala mutations (the LALA double mutation).

Table E1 includes sequence identifiers (SEQ ID NOs) for the amino acid sequences of the B15 heavy chains. Heavy chains for B15 Variants 1-3 and 7 were generated based on the BLV1H12 VH region, while heavy chains for B15 Variants 4-6 and 8 were generated based on humanized variants of the BLV1H12 VH region. The light chain of B15 Variants 1-6 and 8 included the humanized light chain sequence set forth in SEQ ID NO: 181, whereas the light chain of B15 Variant 7 included the bovine light chain sequence encoded by SEQ ID NO: 168.

TABLE E1
Sequence Identifiers (SEQ ID NOs) for Exemplary B15 Heavy Chains

| | Variable Region | | | | | | | |
|---|---|---|---|---|---|---|---|---|
| | | X | | | | | | |
| | | | Cytokine | | | | | |
| B15 Variant No. | | Ascending | Sequence | | | Descending | | Constant |
| (SEQ ID NO) | V1 | Stalk Strand | Region | | | Stalk Strand | V2 | Region |
| B15 Variant 1 | 182 | 183 | 151 | 1 | 186 | 10 | 11 | 187 |
| (189) | | | | | | | | |
| B15 Variant 2 | 182 | 184 | 151 | 1 | 186 | 10 | 11 | 187 |
| (190) | | | | | | | | |
| B15 Variant 3 | 182 | 185 | 151 | 1 | 186 | 10 | 11 | 187 |
| (191) | | | | | | | | |
| B15 Variant 4 | 197 | 183 | 151 | 1 | 186 | 10 | 11 | 188 |
| (192) | | | | | | | | |
| B15 Variant 5 | 197 | 184 | 151 | 1 | 186 | 10 | 11 | 188 |
| (193) | | | | | | | | |
| B15 Variant 6 | 197 | 185 | 151 | 1 | 186 | 10 | 11 | 188 |
| (194) | | | | | | | | |
| B15 Variant 7 | 182 | 159 | 151 | 1 | 186 | 10 | 11 | 196 |
| (198) | | | | | | | | |
| B15 Variant 8 | 197 | 159 | 151 | 1 | 186 | 10 | 11 | 196 |
| (199) | | | | | | | | |

To produce the B15 antibodies, sequences encoding a signal sequence and the B15 heavy chains were chemically synthesized with a 5′ EcoRI site and cloned into pUC57 vectors by GenScript, Inc., using EcoRI and NheI restriction enzymes. A 3′ end NheI site already existed in the synthesized sequence. The expression vectors encoding the heavy chains were then co-transfected into freestyle HEK 293 cells (ThermoScientific) in parallel with a pFUSE expression vector encoding the humanized or bovine light chain. The cells were allowed to grow at 37° C., 8% CO₂, and secreted B15 antibodies were harvested 96 hours after transfection. B15 antibodies were purified by CaptureSelect CH1-XL affinity matrix (ThermoScientific), then concentrated and buffer exchanged into phosphate buffered saline (PBS) using Amicon Ultra-4 centrifugal filters (MW cutoff=10,000 kDa, Millipore Sigma). Harvested B15 antibodies were quantified using Nanodrop based on the molecular weight and extinction coefficient.

The left panel of FIG. 1A shows the crystal structure of BLV1H12. The left panel of FIG. 1B sets forth a schematic depiction of the generated B15 antibodies.

### Example 2 Chimeric IL-15 Fusion Antibody-Induced Receptor Activation and Signaling

Activation of the IL2/15Rβ and γc receptor subunits and STAT5 signaling by chimeric B15 fusion antibodies, generated as described in Example 1, were tested using HEK-Blue IL2 reporter cells (InvivoGen) and analyzed through induction and secretion of the STAT5 inducible alkaline phosphatase (SEAP) reporter gene.

HEK-Blue IL2 reporter cells were placed in suspension by gently rinsing cells twice with pre-warmed phosphate buffered saline (PBS), detaching the cells in the presence of PBS by using a cell scraper, and resuspending the cells in fresh, pre-warmed test medium (DMEM with high glucose and 10% heat-inactivated FBS) to ˜280,000 cells per mL. B15 Variants 1-3, 7, and 8 were 4-fold serially diluted in PBS from 64 nM to 0.25 nM (based on molar concentration of the Fab fragment), and 20 μL of each cytokine dilution was added per well to a 96-well tissue culture treated plate with three replicates per dilution. 50,000 cells were then added to each well and cultured at 37° C., 5% CO₂, for 20 hours. 20 μL of cell culture supernatants from each well containing secreted SEAP were mixed with 180 μL of Quanti-Blue substrate solution at 37° C. for 30 minutes, and the color changes (corresponding to the amount of SEAP secreted) were measured using a Molecular Devices plate reader at 590 nm.

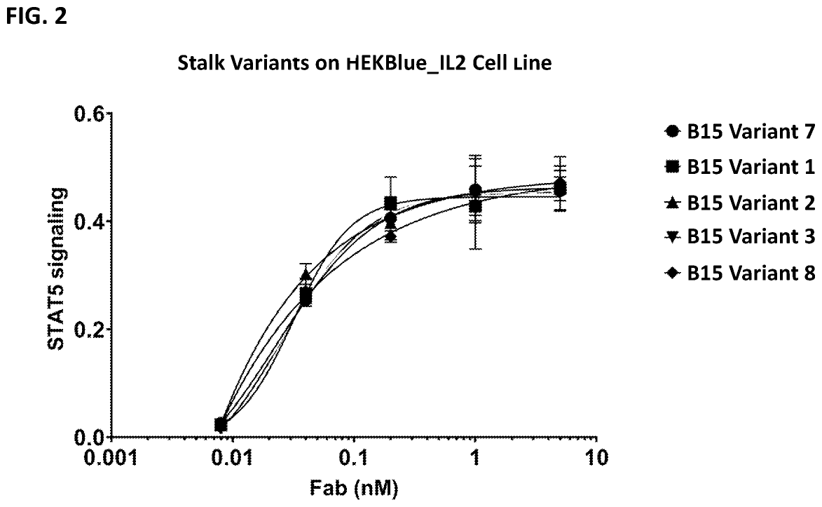

As shown in FIG. 2, B15 Variants 1-3, 7, and 8 induced STAT5 signaling in a dose-dependent manner. Table E2 shows calculated EC50 values for the tested B15 antibodies. These results demonstrate that the generated constructs are able to induce IL-15-mediated signaling activity.

TABLE E2
EC50 Values for B15 Antibody Binding to
IL2/15Rβ and γc Subunits

| | B15 | B15 | B15 | B15 | B15 |
|---|---|---|---|---|---|
| | Based on | Based on | Based on | Based on | Based on |
| | Variant 7 | Variant 1 | Variant 2 | Variant 3 | Variant 8 |
| EC50 | 0.023 | 0.032 | N/A | 0.026 | N/A |
| (nM) | | | | | |

N/A: EC50 could not be determined from binding curve

### Example 3 Generation of Chimeric IL-15 Fusion Antibodies with IL15Rα Sushi Domain

Exemplary chimeric B15 fusion antibodies comprising the extracellular sushi domain of IL15Rα (SEQ ID NO: 2) were generated. B15 Rαsushi antibodies were generated by co-expressing the IL15Rα sushi domain with exemplary B15 antibodies (B15 Variants 3 and 6, as described in Example 1) to produce B15Rαsushi antibodies. Expression vectors encoding these sequences as well as a signal sequence were co-transfected into freestyle HEK 293 cells that were then allowed to grow at 37° C., 8% CO₂. Expressed B15 Rαsushi antibodies were secreted and harvested 96 hours after transfection. B15 Rαsushi antibodies were purified by CaptureSelect CH1-XL affinity matrix (ThermoScientific), then concentrated and buffer exchanged into phosphate buffered saline (PBS) using Amicon Ultra-4 centrifugal filters (MW cutoff=10,000 kDa, Millipore Sigma). Harvested antibodies were quantified using Nanodrop based on the molecular weight and extinction coefficient.

The right panel of FIG. 1A and the middle and right panels of FIG. 1B set forth schematic depictions of the generated B15 Rαsushi antibodies.

### Example 4 Receptor Activation and Signaling by Chimeric IL-15 Rαsushi Fusion Antibodies

Activation of the IL2/15Rβ and γc receptor subunits and STAT5 signaling by chimeric B15 Rαsushi fusion antibodies, generated as described in Example 3, are tested using HEK-Blue IL2 reporter cells (InvivoGen) and analyzed through induction and secretion of the STAT5 inducible alkaline phosphatase (SEAP) reporter gene.

HEK-Blue IL2 reporter cells are prepared as described in Example 2 and are co-cultured with 4-fold serially diluted (from 64 nM to 0.25 nM based on Fab concentration) B15 Rαsushi antibodies at 37° C., 5% CO₂, for 20 hours. 20 μL of cell culture supernatants from each well containing secreted SEAP are mixed with 180 μL of Quanti-Blue substrate solution at 37° C. for 30 minutes, and the color changes (corresponding to the amount of SEAP secreted) are measured using a Tecan plate reader at 590 nm.

### Example 5 Expansion of NK-92 Cells Induced by Chimeric IL-15 Fusion Antibodies

The activity of chimeric B15 fusion antibodies, generated as described in Example 1, and of chimeric B15 Rαsushi fusion antibodies, generated as described in Example 3, are assessed for their ability to expand NK-92 natural killer (NK) cells. NK-92 cells express IL2Ra. IL15Rα, and IL2/15Rβ and γc subunits, and their growth and proliferation are dependent on the exogenous addition of IL2 or IL15 to bind and activate these receptors.

NK-92 cells are maintained in growth medium supplied with 200 U/mL of IL-2. Prior to the expansion assays, NK-92 cells are washed twice with the growth medium without IL-2 to remove residual cell-bound IL-2, and 10,000 cells are seeded per well in a tissue culture treated 96-well plate. These cells are incubated with 2-fold serially diluted (from 1.33 nM to 0.005 nM) IL-2 monomers, IL-15 monomers, B15 antibodies, or B15 Rαsushi antibodies at 37° C., 5% CO₂, for 48 hours. Incubation with half-molar concentrations of the B15 or B15 Rαsushi antibodies are compared to IL-2 and IL-15 monomers. Final NK92 cell number per well are assessed by the reduction of the tetrazolium dye MTT to its insoluble formazan by the presence of metabolically active oxidoreductase enzymes (MTT assay kit, Promega).

### Example 6 Assessment of In Vitro Activity of Chimeric IL-15 Fusion Antibodies in Human PBMCs

The activity of chimeric B15 fusion antibodies, generated as described in Example 1, and of chimeric B15 Rαsushi fusion antibodies, generated as described in Example 3, are assessed for their ability to stimulate NK cells and T cells in human peripheral blood mononuclear cells (PBMCs) in vitro. Both NK cells and T cells express IL15Rα and IL2/15Rβ and γc subunits, and their growth and proliferation are dependent on endogenous or exogenous IL15 to bind and activate the receptors.

Human PBMCs are washed in PBS twice, counted using a hemocytometer, and resuspended in RPMI1640 medium with 10% FBS. 100,000 cells in 100 μL are seeded per well in a tissue culture treated 96-well flat-bottom or a U-bottom (facilitating cell contacts) plate. B15 antibodies and B15 Rαsushi antibodies are 5-fold serially diluted from 500 nM to 0.032 nM in the same medium, and 100 μL of each dilution is added to the corresponding cells to achieve a final concentration from 250 nM to 0.016 nM. Controls are also set up without fusion antibodies added. These cells are incubated at 37° C., 5% CO₂, for 96 hours. After treatment, PBMCs are stained with anti-CD3-FITC (SK7), anti-CD4-PE (OKT4), anti-CD8a-eFluor 450 (SK1), and anti-CD56-APC (AF12-7H3) to gate for the following cell types: CD3+CD4+ T cells, CD3+CD8+ T cells, and NK cells (CD3-CD56+). Intracellular Ki67 as a cell proliferation marker is stained using anti-Ki67-PE-Cy7 (20Raj1) and Foxp3/Transcription Factor Staining Buffer Set (Thermo Fisher Scientific) following the manufacturer's protocol. Stained samples are subsequently analyzed using Novocyte Advanteon Flow Cytometer (Agilent, Santa Clara, CA).

### Example 7 In Vivo Activity of Chimeric IL-15 Fusion Antibodies in Rodents

To examine the in vivo effect of chimeric B15 fusion antibodies on NK cells and T cells in rodents, 18 female 7-9-week-old Fischer344 rats were randomized to six groups of three rats each based on weight on day 0. On day 1 and day 4, rats received either saline vehicle (vehicle, Group 1), control antibody with “no knob” (NK-CTRL, heavy chain set forth in SEQ ID NO: 210, Group 2), engineered IL-15 within the CDR H3 of the bovine VH scaffold (heavy chain of B15 Variant 3, set forth in SEQ ID NO: 191, Group 3), engineered IL-15 within the CDR H3 of the bovine scaffold complexed with the IL-15Rα sushi domain (B15 Variant 3 with Rα, Group 4), engineered IL-15 within the CDR H3 of the humanized scaffold (heavy chain of B15 Variant 6, set forth in SEQ ID NO: 194, Group 5), or engineered IL-15 within the CDR H3 of the humanized scaffold complexed with the IL-15Rα sushi domain (B15 Variant 6 with Ra, Group 6). The sequence of the IL-15Rα sushi domain is set forth in SEQ ID NO: 2. Fusion antibodies of Groups 3-4 and 5-6 contained the light chain derived from bovine V-lambda (encoded by sequence set forth in SEQ ID NO: 168) or humanized V-lambda (SEQ ID NO: 181), respectively. Group 2, the “no knob” negative control without engineered IL-15, contained the bovine VH and VL regions. The constant regions of each antibody were derived from human IgG1 with LALA mutations (SEQ ID NO: 188). Each dose was 0.1 mg/kg intraperitoneally on days 1 and 4, in a volume of approximately 3 mL. Vehicle was dosed in a volume of 3 mL. The groups of rats for this study is shown in Table E3.

TABLE E3
Rodent Study of Chimeric B15 Fusion Antibodies

| | | Treatment Regimen | | | |
|---|---|---|---|---|---|
| Group | n | Agent | mg/kg | Route | Schedule |
| 1 | 3 | Vehicle | — | ip | Days 1, 4 |
| 2 | 3 | NK − Ctrl | 0.1 | ip | Days 1, 4 |
| 3 | 3 | Heavy Chain of B15 | 0.1 | ip | Days 1, 4 |
| | | Variant 3 | | | |
| 4 | 3 | Heavy Chain of B15 | 0.1 | ip | Days 1, 4 |
| | | Variant 3 with Rα | | | |
| 5 | 3 | Heavy Chain of B15 | 0.1 | ip | Days 1, 4 |
| | | Variant 6 | | | |
| 6 | 3 | Heavy Chain of B15 | 0.1 | ip | Days 1, 4 |
| | | Variant 6 with Rα | | | |

Vehicle = saline,
NK − Ctrl = no knob negative control,
ip = intraperitoneal

Weight was monitored daily, and blood was collected sublingually at different time points following the second dose of the fusion antibodies, and a final blood collection by cardiac puncture was performed on day 5. Blood (up to 250 μL) was processed by adding 10× volume of room temperature Ammonium-Chloride-Potassium (ACK) buffer to lyse red blood cells for 5 minutes, followed by a 10× volume of cold PBS to stop the lysis reaction. Samples were centrifuged at 400×g for 5 minutes and washed with PBS. T cells and NK cells were stained by fluorescent antibodies targeting rat CD4 (FITC labeled, clone W3/25, BioLegend) and CD8 (PE labeled, clone OX-8, BioLegend) for T cells and CD161 for NK cells (APC labeled, clone 3.3.3, BioLegend), and live cells were identified by staining with Live/Dead Aqua (Life Technologies).

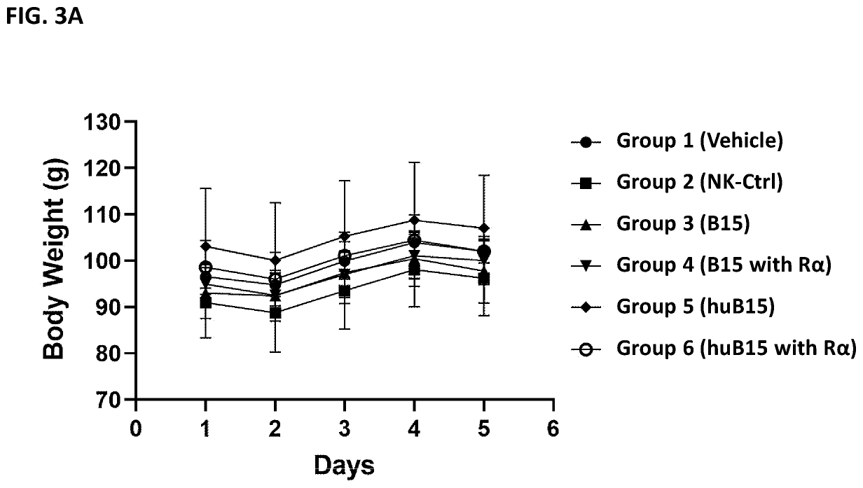

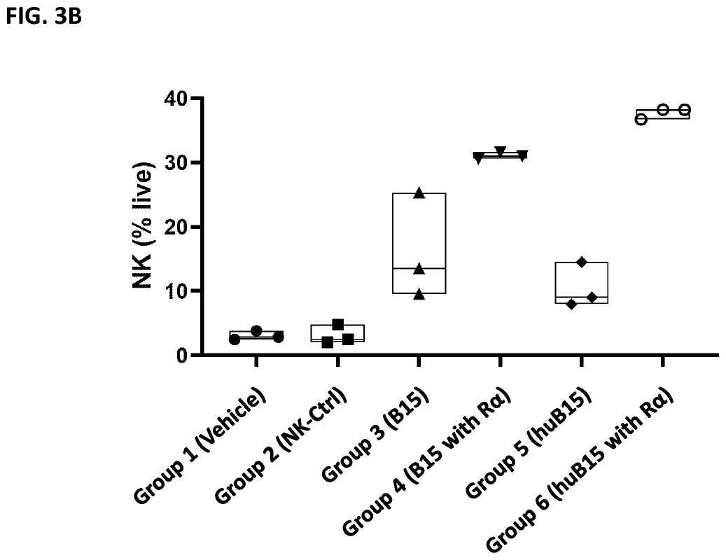

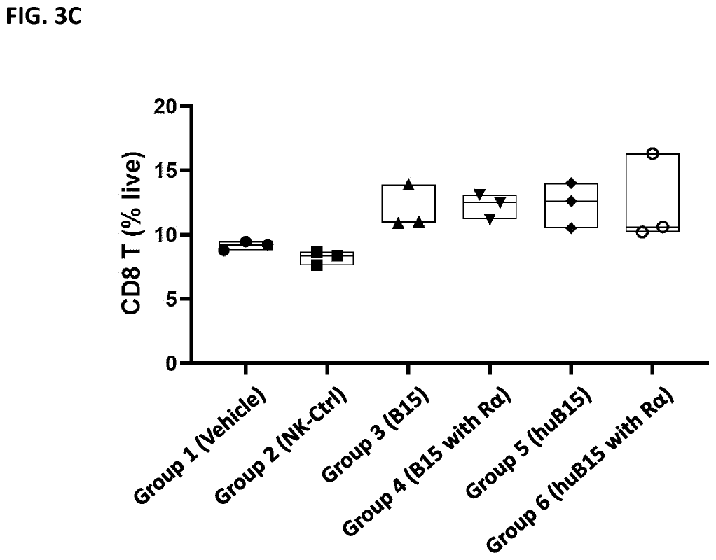

The results showed that none of the engineered fusion antibodies had significant impact on weight of the rats (Table E4 and FIG. 3A). At 15 minutes post last dose, a significant increase in NK cells was observed for Groups 3 to 6, with the groups receiving fusion antibodies with the IL-15Rα chain (Groups 4 and 6) having significantly higher numbers of NK cells (FIG. 3B). At 24 hours post last dose, an increase in CD8 T cells was observed for Groups 3 to 6, with increases comparable between groups receiving or not receiving the IL-15Rα chain (FIG. 3C). Results were comparable for bovine and humanized fusion antibodies.

TABLE E4
Body Weight Changes and Death Events after Chimeric
B15 Fusion Antibodies Administration

| Group | n | Treatment Regimen | Mean BW Nadir | Death |
|---|---|---|---|---|
| 1 | 3 | Vehicle | −1.9% Day 2 | 0 |
| 2 | 3 | NK − CTRL | −2.3% Day 2 | 0 |
| 3 | 3 | Heavy Chain of B15 | −0.7% Day 2 | 0 |
| | | Variant 3 | | |
| 4 | 3 | Heavy Chain of B15 | −2.5% Day 2 | 0 |
| | | Variant 3 with Rα | | |
| 5 | 3 | Heavy Chain of B15 | −2.9% Day 2 | 0 |
| | | Variant 6 | | |
| 6 | 3 | Heavy Chain of B15 | −2.6% Day 2 | 0 |
| | | Variant 6 with Rα | | |

Vehicle = saline,
study duration = 5 days,
Mean BW Nadir = lowest group mean body weight as % change from Day 1

Thus, the engineered chimeric B15 fusion antibodies had biological activity in vivo in rodents, with B15 fusion antibodies containing the IL-15Rα sushi domain having enhanced activity in NK cells.

### Example 8 In Vivo Activity of Chimeric B15 Fusion Antibodies in Non-Human Primates

To examine the in vivo effect of B15 fusion antibodies on NK cells and T cells in non-human primates, 9 male naïve cynomolgus monkeys were randomized to three groups of three monkeys each based on weight (from 2.7 to 4.7 kg) on day 0. On day 1, monkeys received a single dose of either engineered IL-15 within the CDR H3 of the humanized scaffold (heavy chain of B15 Variant 6, SEQ ID NO: 194, Group 1), engineered IL-15 within the CDR H3 of the humanized scaffold complexed with the IL-15Rα sushi domain (Group 2), or humanized control antibody with “no knob” (SEQ ID NO: 211, Group 3). The sequence of the IL-15Rα sushi domain is set forth in SEQ ID NO: 2. Fusion antibodies for all groups contained the light chain derived from humanized V-lambda (SEQ ID NO: 181). The constant regions of each antibody were derived from human IgG1 with “LALA mutations” (SEQ ID NO: 188). The dose was 0.1 mg/kg intravenously administrated on day 1, in a volume that ranged between 1.4 to 2.4 mL depending on body weight. The groups of monkeys for this study are shown in Table E5.

TABLE E5
Non-Human Primate Study of Chimeric B15 Fusion Antibodies

| | | Dose Level | | No. of Male | |
|---|---|---|---|---|---|
| Group | Test Article | (mg/kg) | Route | Animals | Schedule |
| 1 | Heavy Chain | 0.1 | iv | 3 | Day 1 |
| | of B15 | | | | |
| | Variant 6 | | | | |
| 2 | Heavy Chain | 0.1 | iv | 3 | Day 1 |
| | of B15 | | | | |
| | Variant 6 | | | | |
| | with Rα | | | | |
| 3 | NK − Ctrl | 0.1 | iv | 3 | Day 1 |

NK − Ctrl = no knob negative control,
iv = intravenous

Weight was measured the day before administration and on the last day of the experiment. Blood samples (0.5 mL) were collected from the femoral vein at different time points before and following the dose of the fusion antibodies (Days −3, 1, 2, 3, 4, 5, 6, 7, 10, 15, and 22). The samples were collected for evaluation of leukocyte phenotypes by flow cytometry. The samples were accessioned and processed on the day of collection. For each sample, absolute cell count and cell percentage values were calculated per phenotype. A dual platform method was used to determine absolute counts. In this dual approach, the cell percentage values obtained via flow cytometry were each used in conjunction with the absolute leukocyte differential cell counts (i.e., lymphocyte or monocytes) determined by the hematology analyzer to obtain the absolute numbers of each cell type per μL of whole blood for each individual sample. In addition to determining the absolute leukocyte count, the panel of tests contained monoclonal antibodies identifying the cell types in Table E6. Aliquots of the whole blood specimens were stained with predetermined volumes of previously tested and titered monoclonal antibodies specific for each phenotype marker. After staining, the red blood cells in each tube were lysed. The prepared samples were analyzed on BD FACSDiva v8.0.2. For pharmacodynamic analyses, treated monkeys' values were compared to pretreatment values. Fold change (x) in peripheral blood leukocyte counts was determined by comparing the treatment group mean or individual value to the respective pretreatment (Day −3) group mean or individual value.

TABLE E6
Panel of Leukocyte Phenotypes

| Cell Type | Antigen Marker |
|---|---|
| Mature T Cells | CD45⁺CD3⁺ |
| Helper T Cells | CD45⁺CD3⁺CD4⁺ |
| Cytotoxic T Cells | CD45⁺CD3⁺CD8⁺ |
| Natural Killer Cells | CD45⁺CD159a⁺ |
| B Cells | CD45⁺CD20⁺ |
| Monocytes | CD45⁺CD14⁺ |

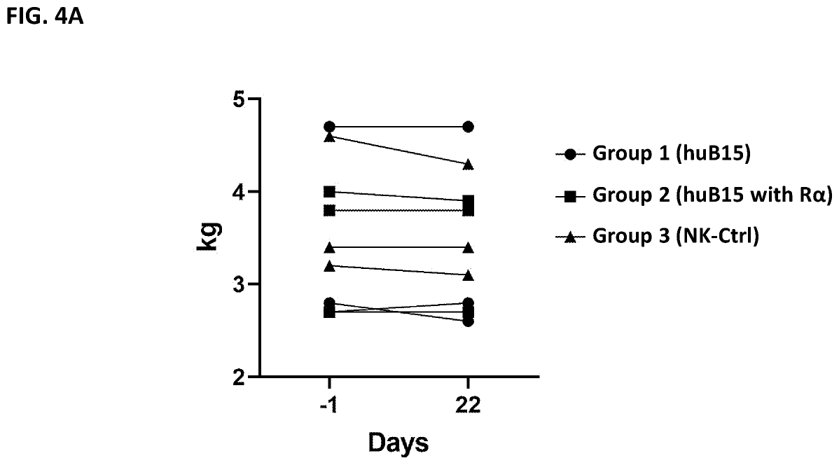

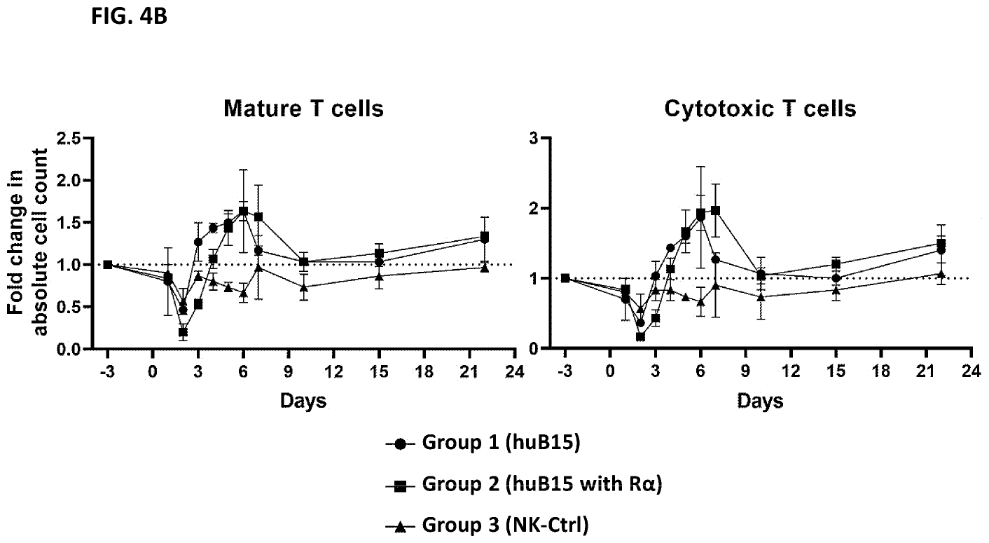

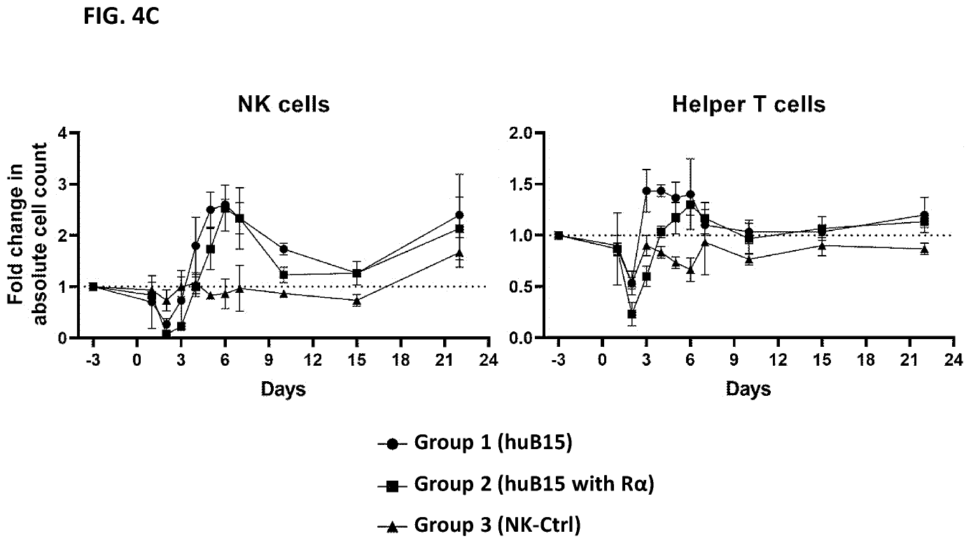

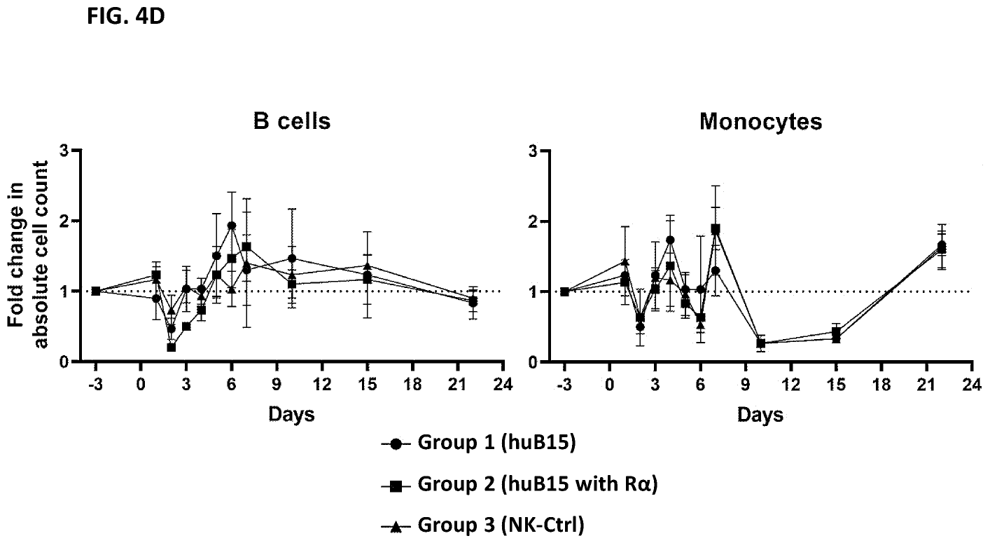

The results showed that none of the engineered antibodies had significant impact on weight of the monkeys (FIG. 4A). On Day 2, there were minimal to moderate decreases in absolute counts of all lymphocyte subtypes evaluated by immunophenotyping (i.e., mature T cells [as low as 0.21×], helper T cells [as low as 0.24×], cytotoxic T cells [as low as 0.17×], NK cells [as low as 0.09×], and B cells [as low as 0.23×]), which resolved by Day 3 (FIG. 4B-4D). On Days 4 or 5 through Day 6 or 7 in animals that received the fusion antibodies (Groups 1 and 2), there were increases in absolute counts of all lymphocyte subtypes evaluated (i.e., mature T cells [up to 1.67×], helper T cells [up to 1.48×], cytotoxic T cells [up to 2.01×], NK cells [up to 2.58×], and B cells [up to 1.83×]), which resolved by Day 7 or 10, while monkeys of Group 3 (control) did not have increased lymphocyte counts (FIG. 4B-4D). The increase in lymphocyte number in monkeys of Group 2 (humanized B15 with IL-15Rα sushi domain) occurred one day later than in monkeys of Group 1 (humanized B15 without IL-15Rα sushi domain) and also resolved one day later (FIG. 4B-4D). The fusion antibodies had no noticeable effect on monocytes in treated monkeys compared to control (FIG. 4D).

Thus, both B15 fusion antibodies, with and without the IL-15Rα sushi domain, enhanced NK and T cell numbers in non-human primates.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| SEQ | | |
|---|---|---|
| ID | | |
| NO | SEQUENCE | ANNOTATION |
| 1 | NWVNVISDLKKIEDLIQSMHIDATLYTESDVHPSC | IL-15 |
| | KVTAMKCFLLELQVISLESGDASIHDTVENLIILA | |
| | NNSLSSNGNVTESGCKECEELEEKNIKEFLQSFVH | |
| | IVQMFINTS | |
| 2 | ITCPPPMSVEHADIWVKSYSLYSRERYICNSGFKR | IL-15-Rαsushi |
| | KAGTSSLTECVLNKATNVAHWTTPSLKCIRDPALV | |
| | HQRPAPP | |
| 3 | atcacctgcccacctccaatgagcgtggagcacgc | IL-15- |
| | agacatctgggtgaagtcttacagcctgtattccc | Rαsushi_GS |
| | gggagagatacatctgcaactctggcttcaagcgg | linker_BLV1H1 |
| | aaggccggcaccagctccctgacagagtgcgtgct | 2 light chain |
| | gaacaaggccaccaatgtggcccactggacaactc | |
| | cttccctgaaatgtattagagaccccgccctggtg | |
| | catcagagacctgccccccctggtggaggcggttc | |
| | aggcggaggtggatcccaggccgtcctgaaccagc | |
| | caagcagcgtctccgggtctctggggcagcgggtc | |
| | tcaatcacctgtagcgggtcttcctccaatgtcgg | |
| | caacggctacgtgtcttggtatcagctgatccctg | |
| | gcagtgccccacgaaccctgatctacggcgacaca | |
| | tccagagcttctggggtccccgatcggttctcagg | |
| | gagcagatccggaaacacagctactctgaccatca | |
| | gctccctgcaggctgaggacgaagcagattatttc | |
| | tgcgcatctgccgaggactctagttcaaatgccgt | |
| | gtttggaagcggcaccacactgacagtcctaggtc | |
| | agcccaaggctgccccctcggtcactctgttcccg | |
| | ccctcctctgaggagcttcaagccaacaaggccac | |
| | actggtgtgtctcataagtgacttctacccgggag | |
| | ccgtgacagtggcctggaaggcagatagcagcccc | |
| | gtcaaggcgggagtggagaccaccacaccctccaa | |
| | acaaagcaacaacaagtacgcggccagcagctatc | |
| | tgagcctgacgcctgagcagtggaagtcccacaga | |
| | agctacagctgccaggtcacgcatgaagggagcac | |
| | cgtggagaagacagtggcccctacagaatgttcat | |
| | aa | |
| 4 | VNGTSQFTCFYNSRANISCVWSQDGALQDTSCQVH | IL2 receptor |
| | AWPDRRRWNQTCELLPVSQASWACNLILGAPDSQK | subunit beta |
| | LTTVDIVTLRVLCREGVRWRVMAIQDFKPFENLRL | |
| | MAPISLQVVHVETHRCNISWEISQASHYFERHLEF | |
| | EARTLSPGHTWEEAPLLTLKQKQEWICLETLTPDT | |
| | QYEFQVRVKPLQGEFTTWSPWSQPLAFRTKPAALG | |
| | KDTIPWLGHLLVGLSGAFGFIILVYLLINCRNTGP | |
| | WLKKVLKCNTPDPSKFFSQLSSEHGGDVQKWLSSP | |
| | FPSSSFSPGGLAPEISPLEVLERDKVTQLLLQQDK | |
| | VPEPASLSSNHSLTSCFTNQGYFFFHLPDALEIEA | |
| | CQVYFTYDPYSEEDPDEGVAGAPTGSSPQPLQPLS | |
| | GEDDAYCTFPSRDDLLLFSPSLLGGPSPPSTAPGG | |
| | SGAGEERMPPSLQERVPRDWDPQPLGPPTPGVPDL | |
| | VDFQPPPELVLREAGEEVPDAGPREGVSFPWSRPP | |
| | GQGEFRALNARLPLNTDAYLSLQELQGQDPTHLV | |
| 5 | caggtccagctgagagagagcggcccttcactggt | BLV1H 12 |
| | caagccatcccagacactgagcctgacatgcacag | heavy chain |
| | caagcgggttttcactgagcgacaaggcagtggga | |
| | tgggtccgacaggcaccaggaaaagccctggaatg | |
| | gctgggcagcatcgataccggcgggaacacagggt | |
| | acaatcccggactgaagagcagactgtccattacc | |
| | aaggacaactctaaaagtcaggtgtcactgagcgt | |
| | gagctccgtcaccacagaggatagtgcaacttact | |
| | attgcacctctgtgcaccaggaaactaagaaatac | |
| | cagagctgtcctgacggctatcgggagagatctga | |
| | ttgcagtaataggccagcttgtggcacatccgact | |
| | gctgtcgcgtgtctgtcttcgggaactgcctgact | |
| | accctgcctgtgtcctactcttatacctacaatta | |
| | tgaatggcatgtggatgtctggggacagggcctgc | |
| | tggtgacagtctctagtgctagc | |
| 6 | atgggatggtcatgtatcatcctttttctagtagc | (Sig Seq- |
| | aactgcaaccggtgtacattcccaggtgcagctgc | VRegion- |
| | gggagtcgggccccagcctgatgaagccgtcacag | CH1CH2CH3) |
| | accctctccctcacctgcacggtctctggatcttc | BLV1 |
| | attgaacgacaagtctgtaggctgggtccgccagg | H12 V in |
| | ctccagggaaggcgctgcagtggctcggtagtgtg | human IgG |
| | gacactagtggaaacacagactataacccaggcct | |
| | gaaatcccggctcagcatcaccaaggacaactcca | |
| | agagccgaatctctcttacagtgactggcatgaca | |
| | actgaagactcggccacatactactgtacttctgt | |
| | gcaccaggaaacaaaaaaataccaaagttgtccgg | |
| | aggattatacttataatccacgttgccctcagcag | |
| | tatggttggagtgactgtgattgtatgggcgatag | |
| | gtttgggggttactgtcgacaggatggttgtagta | |
| | attatagttatacttacaattacgaatggcacgtc | |
| | gatgtctggggccaaggactcctggtcaccgtctc | |
| | ctcagctagcaccaagggcccatcggtcttccccc | |
| | tggcaccctcctccaagagcacctctgggggcaca | |
| | gcggccctgggctgcctggtcaaggactacttccc | |
| | cgaacctgtgacggtctcgtggaactcaggcgccc | |
| | tgaccagcggcgtgcacaccttcccggctgtccta | |
| | cagtcctcaggactctactccctcagcagcgtggt | |
| | gaccgtgccctccagcagcttgggcacccagacct | |
| | acatctgcaacgtgaatcacaagcccagcaacacc | |
| | aaggtggacaagagagttgagcccaaatcttgtga | |
| | caaaactcacacatgcccaccgtgcccagcacctg | |
| | aactcctggggggaccgtcagtcttcctcttcccc | |
| | ccaaaacccaaggacaccctcatgatctcccggac | |
| | ccctgaggtcacatgcgtggtggtggacgtgagcc | |
| | acgaagaccctgaggtcaagttcaactggtacgtg | |
| | gacggcgtggaggtgcataatgccaagacaaagcc | |
| | gcgggaggagcagtacaacagcacgtaccgtgtgg | |
| | tcagcgtcctcaccgtcctgcaccaggactggctg | |
| | aatggcaaggagtacaagtgcaaggtctccaacaa | |
| | agccctcccagcccccategagaaaaccatctcca | |
| | aagccaaagggcagccccgagaaccacaggtgtac | |
| | accctgcccccatcccgggaggagatgaccaagaa | |
| | ccaggtcagcctgacctgcctggtcaaaggcttct | |
| | atcccagcgacatcgccgtggagtgggagagcaat | |
| | gggcagccggagaacaactacaagaccacgcctcc | |
| | cgtgctggactccgacggctccttcttcctctata | |
| | gcaagctcaccgtggacaagagcaggtggcagcag | |
| | gggaacgtcttctcatgctccgtgatgcatgaggc | |
| | tctgcacaaccactacacgcagaagagcctctccc | |
| | tgtccccgggtaaatga | |
| 7 | gaattccaccatgggatggtcatgtatcatccttt | B15 variable |
| | ttctagtagcaactgcaaccggagtacattcccag | region plus |
| | gtgcagctgcgcgagtcgggccccagcctggtgaa | signal peptide |
| | gccgtcacagaccctctcgctcacctgcacggcct | |
| | ctggattctcattgagcgacaaggctgtaggctgg | |
| | gtccgccaggctccagggaaggcgctggagtggct | |
| | cggtagtatagacactggtggaaacacaggctata | |
| | acccaggcctgaaatcccggctcagcatcaccaag | |
| | gacaactccaagagtcaagtctctctgtcagtgag | |
| | cagcgtgacaactgaggactcggccacatactact | |
| | gtacttctgtgcaccaggaaacaaaaaaataccaa | |
| | accggtggatcaaactgggtgaatgtaataagtga | |
| | tttgaaaaaaattgaagatcttattcaatctatgc | |
| | atattgatgctactttatatacggaaagtgatgtt | |
| | caccccagttgcaaagtaacagcaatgaagtgctt | |
| | tctcttggagttacaagttatttcacttgagtccg | |
| | gagatgcaagtattcatgatacagtagaaaatctg | |
| | atcatcctagcaaacaacagtttgtcttctaatgg | |
| | gaatgtaacagaatctggatgcaaagaatgtgagg | |
| | aactggaggaaaaaaatattaaagaatttttgcag | |
| | agttttgtacatattgtccaaatgttcatcaacac | |
| | ttctggttcaggatcctatacttacaattacgaat | |
| | ggcacgtcgatgtctggggccaaggactcctggtc | |
| | accgtctcctcagctagc | |
| 8 | tcacgaattcgcaggccgtcctgaaccagccaagc | BLV1H12 Light |
| | agcgtctccgggtctctggggcagcgggtctcaat | Chain |
| | cacctgtagcgggtcttcctccaatgtcggcaacg | |
| | gctacgtgtcttggtatcagctgatccctggcagt | |
| | gccccacgaaccctgatctacggcgacacatccag | |
| | agcttctggggtccccgatcggttctcagggagca | |
| | gatccggaaacacagctactctgaccatcagctcc | |
| | ctgcaggctgaggacgaagcagattatttctgcgc | |
| | atctgccgaggactctagttcaaatgccgtgtttg | |
| | gaagcggcaccacactgacagtcctggggcagccc | |
| | aagagtcccccttcagtgactctgttcccaccctc | |
| | taccgaggaactgaacggaaacaaggccacactgg | |
| | tgtgtctgatcagcgacttttaccctggatccgtc | |
| | actgtggtctggaaggcagatggcagcacaattac | |
| | taggaacgtggaaactacccgcgcctccaagcagt | |
| | ctaatagtaaatacgccgccagctcctatctgagc | |
| | ctgacctctagtgattggaagtccaaagggtcata | |
| | tagctgcgaagtgacccatgaaggctcaaccgtga | |
| | ctaagactgtgaaaccatccgagtgctcctaggct | |
| | agctggc | |
| 9 | TSVHQETKKYQS | BLV1H12 |
| | | ascending stalk |
| | | region |
| 10 | SYTYNYEWHVDV | BLV1H12 |
| | | decending stalk |
| | | region |
| 11 | WGQGLLVTVSS | V2 alternative |
| | | sequence |
| 12 | QVQLREWGAGLLKPSETLSLTCAVYGGSFSGYYWS | V1 Alternative B |
| | WIRQPPGKGLEWIGEINHSGSTNYNPSLKSRVTIS | sequence of |
| | VDTSKNQFSLKLSSVTAADTAVYYC | VH4-34_Q5RQ6E |
| 13 | QVQLREWGAGLLKPSETLSLTCAVYGGSFSDKYWS | V1 Alternative B |
| | WIRQPPGKGLEWIGEINHSGSTNYNPSLKSRVTIS | sequence of |
| | VDTSKNQFSLKLSSVTAADTAVYYC | VH4-34_CDR1- |
| | | G31DY32K_Q5 |
| | | RQ6E |
| 14 | QVQLREWGAGLLKPSETLSLTCAVYGGSFSGYYWS | V1 Alternative B |
| | WIRQPPGKGLEWIGSINHSGSTNYNPSLKSRVTIS | sequence of |
| | VDTSKNQFSLKLSSVTAADTAVYYC | VH4-34 |
| | | _CDR2- |
| | | E50S_Q5RQ6E |
| 15 | QVQLREWGAGLLKPSETLSLTCAVYGGSFSDKYWS | synthesized: V1 |
| | WIRQPPGKGLEWIGSINHSGSTNYNPSLKSRVTIS | Alternative B |
| | VDTSKNQFSLKLSSVTAADTAVYYC | sequence of |
| | | VH4-34_CDR1- |
| | | G31DY32K_CD |
| | | R2-E50S_Q5RQ6E |
| 16 | QVQLREWGAGLLKPSETLSLTCTASGFSLSDKAVG | synthesized: V1 |
| | WIRQPPGKGLEWIGEINHSGSTNYNPSLKSRVTIS | Alternative B |
| | VDTSKNQFSLKLSSVTAADTAVYYC | sequence of |
| | | VH4-34_CDR1- |
| | | Cow_Q5RQ6E |
| 17 | QVQLREWGAGLLKPSETLSLTCAVYGGLGSIDTGG | synthesized: V1 |
| | NTGSFSGYYWSWIRQPPGKGLEWYNPSLKSRVTIS | Alternative B |
| | VDTSKNQFSLKLSSVTAADTAVYYC | sequence of |
| | | VH4-34_CDR2- |
| | | Cow_Q5RQ6E |
| 18 | QVQLREWGAGLLKPSETLSLTCTASGFSLSDKAV | synthesized: V1 |
| | GWIRQPPGKGLEWIGSINHSGSTNYNPSLKSRVTI | Alternative B |
| | SVDTSKNQFSLKLSSVTAADTAVYYC | sequence of |
| | | VH4-34_CDR1- |
| | | Cow_CDR2- |
| | | E50S_ |
| | | Q5RQ6E |
| 19 | QVQLREWGAGLLKPSETLSLTCTASGFSLSDKAVG | synthesized: V1 |
| | WIRQPPGKGLEWLGSIDTGGNTGYNPSLKSRVTIS | Alternative N |
| | VDTSKNQFSLKLSSVTAADTAVYYC | sequence of |
| | | VH4-34_CDR1- |
| | | Cow_CDR2- |
| | | Cow_Q |
| | | 5RQ6E |
| 20 | WGHGTAVTVSS | V2 alternative |
| | | sequence |
| 21 | WGKGTTVTVSS | V2 alternative |
| | | sequence |
| 22 | WGKGTTVTVSS | V2 alternative |
| | | sequence |
| 23 | WGRGTLVTVSS | V2 alternative |
| | | sequence |
| 24 | WGKGTTVTVSS | V2 alternative |
| | | sequence |
| 25 | SVHQETKKYQSCPDGYRERSDCSNRPACGTSDCCR | Synthesized: |
| | VSVFGNCLTTLPVSYSYTYNYEWHVD | ultralong CDR3 |
| | | sequence |
| | | (BLV1H12) |
| 26 | QVQLRESGPSLVKPSQTLSLTCTASGFSLSDKAVG | BLV1H12 |
| | WVRQAPGKALEWLGSIDTGGNTGYNPGLKSRLSIT | Heavy Chain |
| | KDNSKSQVSLSVSSVTTEDSATYYCTSVHQETKKY | |
| | QSCPDGYRERSDCSNRPACGTSDCCRVSVFGNCLT | |
| | TLPVSYSYTYNYEWHVDVWGQGLLVTVSSASTTAP | |
| | KVYPLSSCCGDKSSSTVTLGCLVSSYMPEPVTVTW | |
| | NSGALKSGVHTFPAVLQSSGLYSLSSMVTVPGSTS | |
| | GQTFTCNVAHPASSTKVDKAVEPKSCDGS | |
| 27 | QAVLNQPSSVSGSLGQRVSITCSGSSSNVGNGYVS | BLV1H12 Light |
| | WYQLIPGSAPRTLIYGDTSRASGVPDRESGSRSGN | Chain |
| | TATLTISSLQAEDEADYFCASAEDSSSNAVFGSG | |
| | TTLTVLGQPKSPPSVTLFPPSTEELNGNKATLVCL | |
| | ISDFYPGSVTVVWKADGSTITRNVETTRASKQSNS | |
| | KYAASSYLSLTSSDWKSKGSYSCEVTHEGSTVTKT | |
| | VKPSECS | |
| 28 | QVQLRESGPSLVQPSQTLSLTCTASGFSLSDKAVG | BLV5B8 heavy |
| | WVRQAPGKALEWLGSIDTGGSTGYNPGLKSRLSIT | chain |
| | KDNSKSQVSLSVSSVTTEDSATYYCTTVHQETRKT | |
| | CSDGYIAVDSCGRGQSDGCVNDCNSCYYGWRNCRR | |
| | QPAIHSYEFHVDAWGRGLLVTVSSASTTAPKVYPL | |
| | SSCCGDKSSSTVTLGCLVSSYMPEPVTVTWNSGAL | |
| | KSGVHTFPAVLQSSGLYSLSSMVTVPGSTSGQTFT | |
| | CNVAHPASSTKVDKAVEPKSCDGS | |
| 29 | QAVLNQPSSVSGSLGQRVSITCSGSSSNVGNGYVS | BLV5B8 light |
| | WYQLIPGSAPRTLIYGDTSRASGVPDRESGSRSGN | chain |
| | TATLTISSLQAEDEADYFCASAEDSSSNAVFGSGT | |
| | TLTVLGQPKSPPSVTLFPPSTEELNGNKATLVCLI | |
| | SDFYPGSVTVVWKADGSTITRNVETTRASKQSNSK | |
| | YAASSYLSLTSSDWKSKGSYSCEVTHEGSTVTKTV | |
| | KPSECS | |
| 30 | TVHQETRKTCSDGYIAVDSCGRGQSDGCVNDCNSC | BLV5B8 CDR3 |
| | YYGWRNCRRQPAIHSYEFHVD | |
| 31 | SVTQRTHVSRSCPDGCSDGDGCVDGCCCSAYRCYT | BLV5D3 CDR3 |
| | PGVRDLSCTSYSITYTYEWNVD | |
| 32 | TVHQKTTRKTCCSDAYRYDSGCGSGCDCCGADCYV | BLV8C11 |
| | FGACTFGLDSSYSYIYIYQWYVD | CDR3 |
| 33 | TVHQIFCPDGYSYGYGCGYGYGCSGYDCYGYGGYG | BF4E9 CDR3 |
| | YGGYGGYSSYSYSYSYEYYGD | |
| 34 | TVHPSPDGYSYGYGCGYGYGCSGYDCYGYGGYGYG | BF1H1 CDR3 |
| | GYGGYSSYSYSYS | |
| 35 | TVHQIRCPDGYGYGYGCGYGSYGYSGYDCYGYGGY | F18 CDR3 |
| | GGYGGYGGYSSYS | |
| 36 | TTVHQ | Ascending Stalk |
| | | Strand |
| 37 | TSVHQ | Ascending Stalk |
| | | Strand |
| 38 | SSVTQ | Ascending Stalk |
| | | Strand |
| 39 | STVHQ | Ascending Stalk |
| | | Strand |
| 40 | ATVRQ | Ascending Stalk |
| | | Strand |
| 41 | TTVYQ | Ascending Stalk |
| | | Strand |
| 42 | SPVHQ | Ascending Stalk |
| | | Strand |
| 43 | ATVYQ | Ascending Stalk |
| | | Strand |
| 44 | TAVYQ | Ascending Stalk |
| | | Strand |
| 45 | TNVHQ | Ascending Stalk |
| | | Strand |
| 46 | ATVHQ | Ascending Stalk |
| | | Strand |
| 47 | STVYQ | Ascending Stalk |
| | | Strand |
| 48 | TIVHQ | Ascending Stalk |
| | | Strand |
| 49 | AIVYQ | Ascending Stalk |
| | | Strand |
| 50 | TTVFQ | Ascending Stalk |
| | | Strand |
| 51 | AAVFQ | Ascending Stalk |
| | | Strand |
| 52 | GTVHQ | Ascending Stalk |
| | | Strand |
| 53 | ASVHQ | Ascending Stalk |
| | | Strand |
| 54 | TAVFQ | Ascending Stalk |
| | | Strand |
| 55 | ATVFQ | Ascending Stalk |
| | | Strand |
| 56 | AAAHQ | Ascending Stalk |
| | | Strand |
| 57 | VWYQ | Ascending Stalk |
| | | Strand |
| 58 | GTVFQ | Ascending Stalk |
| | | Strand |
| 59 | TAVHQ | Ascending Stalk |
| | | Strand |
| 60 | ITVHQ | Ascending Stalk |
| | | Strand |
| 61 | ITAHQ | Ascending Stalk |
| | | Strand |
| 62 | VTVHQ | Ascending Stalk |
| | | Strand |
| 63 | AAVHQ | Ascending Stalk |
| | | Strand |
| 64 | GTVYQ | Ascending Stalk |
| | | Strand |
| 65 | TTVLQ | Ascending Stalk |
| | | Strand |
| 66 | TTTHQ | Ascending Stalk |
| | | Strand |
| 67 | TTDYQ | Ascending Stalk |
| | | Strand |
| 68 | QLQLQESGPGLVKPSETLSLTCTVSGGSISSSSYY | Human heavy |
| | WGWIRQPPGKGLEWIGSIYYSGSTYYNPSLKSRVT | chain variable |
| | ISVDTSKNQFSLKLSSVTAADTAVYYCAR | region s |
| | | equence |
| | | VH4-39 |
| 69 | QVQLQESGPGLVKPSETLSLTCTVSGGSISSYYWS | Human heavy |
| | WIRQPPGKGLEWIGYIYYSGSTNYNPSLKSRVTIS | chain variable |
| | VDTSKNQFSLKLSSVTAADTAVYYCA | region s |
| | | equence |
| | | 4-59*03 |
| 70 | QVQLQQWGAGLLKPSETLSLTCAVYGGSFSGYYWS | Human heavy |
| | WIRQPPGKGLEWIGEINHSGSTNYNPSLKSRVTIS | chain variable |
| | VDTSKNQFSLKLSSVTAADTAVYYCAR | region s |
| | | equence |
| | | 4-34*02 |
| 71 | QVQLQESGPGLVKPSQTLSLTCAVYGGSFSGYYWS | Human heavy |
| | WIRQPPGKGLEWIGEINHSGSTNYNPSLKSRVTIS | chain variable |
| | VDTSKNQFSLKLSSVTAADTAVYYCAR | region s |
| | | equence |
| | | 4-34*09 |
| 72 | TSVHQETKKYQ | Ascending Stalk |
| | | Strand |
| 73 | VHQETKKYQ | Ascending Stalk |
| | | Strand |
| 74 | IHSYEF | Ascending Stalk |
| | | Strand |
| 75 | SYEF | Ascending Stalk |
| | | Strand |
| 76 | YTYNYE | Descending |
| | | Stalk Strand |
| 77 | YTYNYEW | Descending |
| | | Stalk Strand |
| 78 | SYTYNYEW | Descending |
| | | Stalk Strand |
| 79 | TYNYEW | Descending |
| | | Stalk Strand |
| 80 | SYTY | Descending |
| | | Stalk Strand |
| 81 | GSKHRLRDYFLYNE | Ascending Stalk |
| | | Strand |
| 82 | GSKHRLRDYFLYN | Ascending Stalk |
| | | Strand |
| 83 | GSKHRLRDYFLY | Ascending Stalk |
| | | Strand |
| 84 | GSKHRLRDYFL | Ascending Stalk |
| | | Strand |
| 85 | GSKHRLRDYF | Ascending Stalk |
| | | Strand |
| 86 | GSKHRLRDY | Ascending Stalk |
| | | Strand |
| 87 | GSKHRLRD | Ascending Stalk |
| | | Strand |
| 88 | EAGGPDYRNGYNY | Ascending Stalk |
| | | Strand |
| 89 | EAGGPDYRNGYN | Ascending Stalk |
| | | Strand |
| 90 | EAGGPDYRNGY | Ascending Stalk |
| | | Strand |
| 91 | EAGGPDYRNG | Ascending Stalk |
| | | Strand |
| 92 | EAGGPDYRN | Ascending Stalk |
| | | Strand |
| 93 | EAGGPDYR | Ascending Stalk |
| | | Strand |
| 94 | EAGGPDY | Ascending Stalk |
| | | Strand |
| 95 | EAGGPD | Ascending Stalk |
| | | Strand |
| 96 | EAGGPIWHDDVKY | Ascending Stalk |
| | | Strand |
| 97 | EAGGPIWHDDVK | Ascending Stalk |
| | | Strand |
| 98 | EAGGPIWHDDV | Ascending Stalk |
| | | Strand |
| 99 | EAGGPIWHDD | Ascending Stalk |
| | | Strand |
| 100 | EAGGPIWHD | Ascending Stalk |
| | | Strand |
| 101 | EAGGPIWH | Ascending Stalk |
| | | Strand |
| 102 | EAGGPIW | Ascending Stalk |
| | | Strand |
| 103 | EAGGPI | Ascending Stalk |
| | | Strand |
| 104 | GTDYTIDDQGI | Ascending Stalk |
| | | Strand |
| 105 | GTDYTIDDQG | Ascending Stalk |
| | | Strand |
| 106 | GTDYTIDDQ | Ascending Stalk |
| | | Strand |
| 107 | GTDYTIDD | Ascending Stalk |
| | | Strand |
| 108 | GTDYTID | Ascending Stalk |
| | | Strand |
| 109 | GTDYTI | Ascending Stalk |
| | | Strand |
| 110 | DKGDSDYDYNL | Ascending Stalk |
| | | Strand |
| 111 | DKGDSDYDYN | Ascending Stalk |
| | | Strand |
| 112 | DKGDSDYDY | Ascending Stalk |
| | | Strand |
| 113 | DKGDSDYD | Ascending Stalk |
| | | Strand |
| 114 | DKGDSDY | Ascending Stalk |
| | | Strand |
| 115 | DKGDSD | Ascending Stalk |
| | | Strand |
| 116 | YGPNYEEWGDYLATLDV | Ascending Stalk |
| | | Strand |
| 117 | GPNYEEWGDYLATLDV | Ascending Stalk |
| | | Strand |
| 118 | PNYEEWGDYLATLDV | Ascending Stalk |
| | | Strand |
| 119 | NYEEWGDYLATLDV | Ascending Stalk |
| | | Strand |
| 120 | YEEWGDYLATLDV | Ascending Stalk |
| | | Strand |
| 121 | EEWGDYLATLDV | Ascending Stalk |
| | | Strand |
| 122 | YDFYDGYYNYHYMDV | Descending |
| | | Stalk Strand |
| 123 | DFYDGYYNYHYMDV | Descending |
| | | Stalk Strand |
| 124 | FYDGYYNYHYMDV | Descending |
| | | Stalk Strand |
| 125 | YDGYYNYHYMDV | Descending |
| | | Stalk Strand |
| 126 | DGYYNYHYMDV | Descending |
| | | Stalk Strand |
| 127 | GYYNYHYMDV | Descending |
| | | Stalk Strand |
| 128 | YYNYHYMDV | Descending |
| | | Stalk Strand |
| 129 | YDFNDGYYNYHYMDV | Descending |
| | | Stalk Strand |
| 130 | DFYDGYYNYHYMDV | Descending |
| | | Stalk Strand |
| 131 | FYDGYYNYHYMDV | Descending |
| | | Stalk Strand |
| 132 | YDGYYNYHYMDV | Descending |
| | | Stalk Strand |
| 133 | DGYYNYHYMDV | Descending |
| | | Stalk Strand |
| 134 | GYYNYHYMDV | Descending |
| | | Stalk Strand |
| 135 | QGIRYQGSGTFWYFDV | Descending |
| | | Stalk Strand |
| 136 | GIRYQGSGTFWYFDV | Descending |
| | | Stalk Strand |
| 137 | IRYQGSGTFWYFDV | Descending |
| | | Stalk Strand |
| 138 | RYQGSGTFWYFDV | Descending |
| | | Stalk Strand |
| 139 | YQGSGTFWYFDV | Descending |
| | | Stalk Strand |
| 140 | QGSGTFWYFDV | Descending |
| | | Stalk Strand |
| 141 | GSGTFWYFDV | Descending |
| | | Stalk Strand |
| 142 | SGTFWYFDV | Descending |
| | | Stalk Strand |
| 143 | GTFWYFDV | Descending |
| | | Stalk Strand |
| 144 | YNLGYSYFYYMDG | Descending |
| | | Stalk Strand |
| 145 | NLGYSYFYYMDG | Descending |
| | | Stalk Strand |
| 146 | LGYSYFYYMDG | Descending |
| | | Stalk Strand |
| 147 | GYSYFYYMDG | Descending |
| | | Stalk Strand |
| 148 | YSYFYYMDG | Descending |
| | | Stalk Strand |
| 149 | SYFYYMDG | Descending |
| | | Stalk Strand |
| 150 | GS | Linker |
| 151 | GGS | Linker |
| 152 | GGSGGS | Linker |
| 153 | GGSGGSGGS | Linker |
| 154 | GGGGS | Linker |
| 155 | tcacgaattcgcaggccgtcctgaaccagccaagc | human light |
| | agcgtctccgggtctctggggcagcgggtctcaat | chain lambda |
| | cacctgtagcgggtcttcctccaatgtcggcaacg | region |
| | gctacgtgtcttggtatcagctgatccctggcagt | |
| | gccccacgaaccctgatctacggcgacacatccag | |
| | agcttctggggtccccgatcggttctcagggagca | |
| | gatccggaaacacagctactctgaccatcagctcc | |
| | ctgcaggctgaggacgaagcagattatttctgcgc | |
| | atctgccgaggactctagttcaaatgccgtgtttg | |
| | gaagcggcaccacactgacagtcctaggtcagccc | |
| | aaggctgccccctcggtcactctgttcccgccctc | |
| | ctctgaggagcttcaagccaacaaggccacactgg | |
| | tgtgtctcataagtgacttctacccgggagccgtg | |
| | acagtggcctggaaggcagatagcagccccgtcaa | |
| | ggcgggagtggagaccaccacaccctccaaacaaa | |
| | gcaacaacaagtacgcggccagcagctatctgagc | |
| | ctgacgcctgagcagtggaagtcccacagaagcta | |
| | cagctgccaggtcacgcatgaagggagcaccgtgg | |
| | agaagacagtggcccctacagaatgttcataa | |
| 156 | QSVLTQPPSVSAAPGQKVTISCSGSSSNIGNNYVS | human VL1-51 |
| | WYQQLPGTAPKLLIYDNNKRPSGIPDRFSGSKSGT | |
| | SATLGITGLQTGDEADYYCASAEDSSSNAVFGSGT | |
| | TLTVLGQPKAAPSVTLFPPSSEELQANKATLVCLI | |
| | SDFYPGAVTVAWKADSSPVKAGVETTTPSKQSNNK | |
| | YAASSYLSLTPEQWKSHRSYSCQVTHEGSTVEKTV | |
| | APTECS | |
| 157 | tgtacttetgtgcaccaggaaacaaaaaaatacca | BLV1H12 |
| | aacc | ascending stalk |
| | | region |
| 158 | TSVHQETKKYQT | BLV1H12 |
| | | ascending stalk |
| | | region |
| 159 | CTSVHQETKKYQT | BLV1H12 |
| | | ascending stalk |
| | | region |
| 160 | tcctatacttacaattacgaatggcacgtcgatgt | Descending stalk |
| | ctgg | region |
| 161 | SYTYNYEWHVDVW | Descending stalk |
| | | region |
| 162 | tgtccggaggattatacttataatccacgttgccc | BLV1H12 knob |
| | tcagcagtatggttggagtgactgtgattgtatgg | sequence |
| | gcgataggtttgggggttactgtcgacaggatggt | |
| | tgtagtaattat | |
| 163 | ggtggatca | Coding |
| | | sequencing for |
| | | N-terminal |
| | | GGS |
| | | linker |
| 164 | ggttcagga | Coding sequence |
| | | for C-terminal |
| | | GSG |
| | | linker |
| 165 | APTSSSTKKTQLQLEHLLLDLQMILNGINNYKNPK | IL2 |
| | LTRMLTFKFYMPKKATELKHLQCLEEELKPLEEVL | |
| | NLAQSKNFHLRPRDLISNINVIVLELKGSETTFMC | |
| | EYADETATIVEFLNRWITFCQSIISTLT | |
| 166 | gcacctacttcaagttctacaaagaaaacacagct | IL2 coding |
| | acaactggagcatttactgctggatttacagatga | sequence |
| | ttttgaatggaattaataattacaagaatcccaaa | |
| | ctcaccaggatgctcacatttaagttttacatgcc | |
| | caagaaggccacagaactgaaacatcttcagtgtc | |
| | tagaagaagaactcaaacctctggaggaagtgcta | |
| | aatttagctcaaagcaaaaactttcacttaagacc | |
| | cagggacttaatcagcaatatcaacgtaatagttc | |
| | tggaactaaagggatctgaaacaacattcatgtgt | |
| | gaatatgctgatgagacagcaaccattgtagaatt | |
| | tctgaacagatggattaccttttgtcaaagcatca | |
| | tctcaacactgact | |
| 167 | caggtgcagctgcgggagtcgggccccagcctgat | Chimeric |
| | gaagccgtcacagaccctctccctcacctgcacgg | ultralong bovine |
| | tctctggatcttcattgaacgacaagtctgtaggc | heavy chain |
| | tgggtccgccaggctccagggaaggcgctgcagtg | sequence |
| | gctcggtagtgtggacactagtggaaacacagact | |
| | ataacccaggcctgaaatcccggctcagcatcacc | |
| | aaggacaactccaagagccgaatctctcttacagt | |
| | gactggcatgacaactgaagactcggccacatact | |
| | actgtacttctgtgcaccaggaaacaaaaaaatac | |
| | caaagttgtccggaggattatacttataatccacg | |
| | ttgccctcagcagtatggttggagtgactgtgatt | |
| | gtatgggcgataggtttgggggttactgtcgacag | |
| | gatggttgtagtaattatagttatacttacaatta | |
| | cgaatggcacgtcgatgtctggggccaaggactcc | |
| | tggtcaccgtctcctcagctagc | |
| 168 | caggccgtcctgaaccagccaagcagcgtctccgg | BLV1H12 Light |
| | gtctctggggcagegggtctcaatcacctgtagcg | Chain- |
| | ggtcttcctccaatgtcggcaacggctacgtgtct | |
| | tggtatcagctgatccctggcagtgccccacgaac | |
| | cctgatctacggcgacacatccagagcttctgggg | |
| | tccccgatcggttctcagggagcagatccggaaac | |
| | acagctactctgaccatcagctccctgcaggctga | |
| | ggacgaagcagattatttctgcgcatctgccgagg | |
| | actctagttcaaatgccgtgtttggaagcggcacc | |
| | acactgacagtcctaggtcagcccaaggctgcccc | |
| | cteggtcactctgttcccgccctcctctgaggagc | |
| | ttcaagccaacaaggccacactggtgtgtctcata | |
| | agtgacttctacccgggagccgtgacagtggcctg | |
| | gaaggcagatagcagccccgtcaaggcgggagtgg | |
| | agaccaccacaccctccaaacaaagcaacaacaag | |
| | tacgcggccagcagctatctgagcctgacgcctga | |
| | gcagtggaagtcccacagaagctacagctgccagg | |
| | tcacgcatgaagggagcaccgtggagaagacagtg | |
| | gcccctacagaatgttcataa | |
| 169 | cagctgcagctgcaggagtcgggcccaggactggt | Human heavy |
| | gaagccttcggagaccctgtccctcacctgcactg | chain variable |
| | tctctggtggctccatcagcagtagtagttactac | region sequence |
| | tggggctggatccgccagcccccagggaaggggct | 4-39 |
| | ggagtggattgggagtatctattatagtgggagca | |
| | cctactacaacccgtccctcaagagtcgagtcacc | |
| | atatccgtagacacgtccaagaaccagttctccct | |
| | gaagctgagctctgtgaccgccgcagacacggctg | |
| | tgtattactgtgcgagacacacagtgagggg | |
| 170 | caggtgcagctgcaggagtcgggcccaggactggt | Human heavy |
| | gaagccttcggagaccctgtccctcacctgcactg | chain variable |
| | tctctggtggctccatcagtagttactactggagc | region sequence |
| | tggatccggcagcccccagggaagggactggagtg | 4-59*03 |
| | gattgggtatatctattacagtgggagcaccaact | |
| | acaacccctccctcaagagtcgagtcaccatatca | |
| | gtagacacgtccaagaaccaattctccctgaagct | |
| | gagctctgtgaccgctgcggacacggccgtgtatt | |
| | actgtgcg | |
| 171 | caggtgcagctgcaggagtcgggcccaggactggt | Human heavy |
| | gaagccttcacagaccctgtccctcacctgcgctg | chain variable |
| | tctatggtgggtccttcagtggttactactggagc | region sequence |
| | tggatccgccagcccccagggaagggactggagtg | 4-34*09 |
| | gattggggaaatcaatcatagtggaagcaccaact | |
| | acaacccgtccctcaagagtcgagttaccatatca | |
| | gtagacacgtctaagaaccagttctccctgaagct | |
| | gagctctgtgactgccgcggacacggccgtgtatt | |
| | actgtgcgaga | |
| 172 | caggtgcagctacaacagtggggcgcaggactgtt | Human heavy |
| | gaagccttcggagaccctgtccctcacctgcgctg | chain variable |
| | tctatggtgggtccttcagtggttactactggagc | region sequence |
| | tggatccgccagcccccagggaaggggctggagtg | 4-34*02 |
| | gattggggaaatcaatcatagtggaagcaccaact | |
| | acaacccgtccctcaagagtcgagtcaccatatca | |
| | gtagacacgtccaagaaccagttctccctgaagct | |
| | gagctctgtgaccgccgcggacacggctgtgtatt | |
| | actgtgcgag | |
| 173 | QSVLTQPPSASGTPGQRVTISCSGSSSNIGSNYVY | Human germline |
| | WYQQLPGTAPKLLIYRNNQRPSGVPDRFSGSKSGT | light chain |
| | SASLAISGLRSEDEADYYCAAWDDSLSG | variable |
| | | region |
| | | sequence VL1- |
| | | 47 |
| 174 | QSVLTQPPSVSGAPGQRVTISCTGSSSNIGAGYDV | Human germline |
| | HWYQQLPGTAPKLLIYGNSNRPSGVPDRFSGSKSG | light chain |
| | TSASLAITGLQAEDEADYYCQSYDSSLSG | variable |
| | | region |
| | | sequence VL1- |
| | | 40*1 |
| 175 | QSVLTQPPSVSAAPGQKVTISCSGSSSNIGNNYVS | Human germline |
| | WYQQLPGTAPKLLIYDNNKRPSGIPDRFSGSKSGT | light chain |
| | SATLGITGLQTGDEADYYCGTWDSSLSA | variable |
| | | region |
| | | sequence VL1- |
| | | 51*01 |
| 176 | QSALTQPPSVSGSPGQSVTISCTGTSSDVGSYNRV | Human germline |
| | SWYQQPPGTAPKLMIYEVSNRPSGVPDRFSGSKSG | light chain |
| | NTASLTISGLQAEDEADYYCSSYTSSSTF | variable |
| | | region |
| | | sequence VL2- |
| | | 18*02 |
| 177 | cagtctgtgctgactcagccaccctcagcgtctgg | Human germline |
| | gacccccgggcagagggtcaccatctcttgttctg | light chain |
| | gaagcagctccaacatcggaagtaattatgtatac | variable |
| | tggtaccagcagctcccaggaacggcccccaaact | region |
| | cctcatctataggaataatcagcggccctcagggg | sequence VL1- |
| | tccctgaccgattctctggctccaagtctggcacc | 47 |
| | tcagcctccctggccatcagtgggctccggtccga | |
| | ggatgaggctgattattactgtgcagcatgggatg | |
| | acagcctgagtggtcc | |
| 178 | cagtctgtgctgacgcagccgccctcagtgtctgg | Human germline |
| | ggccccagggcagagggtcaccatctcctgcactg | light chain |
| | ggagcagctccaacatcggggcaggttatgatgta | variable |
| | cactggtaccagcagcttccaggaacagcccccaa | region |
| | actcctcatctatggtaacagcaatcggccctcag | sequence VL1- |
| | gggtccctgaccgattctctggctccaagtctggc | 40*1 |
| | acctcagcctccctggccatcactgggctccaggc | |
| | tgaggatgaggctgattattactgccagtcctatg | |
| | acagcagcctgagtggttc | |
| 179 | cagtctgtgttgacgcagccgccctcagtgtctgc | Human germline |
| | ggccccaggacagaaggtcaccatctcctgctctg | light chain |
| | gaagcagctccaacattgggaataattatgtatcc | variable |
| | tggtaccagcagctcccaggaacagcccccaaact | region |
| | cctcatttatgacaataataagcgaccctcaggga | |
| | ttcctgaccgat | |
| | tctctggctccaagtctggcacgtcagccaccctg | sequence VL1- |
| | ggcatcaccggactccagactggggacgaggccga | 51*01 |
| | ttattactgcggaacatgggatagcagcctgagtg | |
| | ctgg | |
| 180 | cagtctgccctgactcagcctccctccgtgtccgg | Human germline |
| | gtctcctggacagtcagtcaccatctcctgcactg | light chain |
| | gaaccagcagtgacgttggtagttataaccgtgtc | variable |
| | tcctggtaccagcagcccccaggcacagcccccaa | region |
| | actcatgatttatgaggtcagtaatcggccctcag | sequence VL2- |
| | gggtccctgatcgcttctctgggtccaagtctggc | 18*02 |
| | aacacggcctccctgaccatctctgggctccaggc | |
| | tgaggacgaggctgattattactgcagctcatata | |
| | caagcagcagcactttc | |
| 181 | QAVLNQPSSVSGSLGQKVTISCSGSSSNIGNNYVS | B15 Humanized |
| | WYQQLPGTAPKLLIYGDTKRPSGIPDRFSGSKSGT | Light Chain |
| | SATLGITGLQTGDEADYYCASAEDSSSNAVFGSGT | |
| | TLTVLGQPKAAPSVTLFPPSSEELQANKATLVCLI | |
| | SDFYPGAVTVAWKADSSPVKAGVETTTPSKQSNNK | |
| | YAASSYLSLTPEQWKSHRSYSCQVTHEGSTVEKTV | |
| | APTECS | |
| 182 | QVQLRESGPSLVKPSQTLSLTCTASGFSLSDKAVG | B15 V1 Region |
| | WVRQAPGKALEWLGSIDTGGNTGYNPGLKSRLSIT | |
| | KDNSKSQVSLSVSSVTTEDSATYY | |
| 183 | CSTVHQETKKYQT | Ascending Stalk |
| | | Region |
| 184 | CATVHQETKKYQT | Ascending Stalk |
| | | Region |
| 185 | CTTVYQETKKYQT | Ascending Stalk |
| | | Region |
| 186 | GSG | Linker |
| 187 | ASTKGPSVFPLAPSSKSTSGGTAALGCLVKDYFPE | Human IgG1 |
| | PVTVSWNSGALTSGVHTFPAVLQSSGLYSLSSVVT | heavy constant |
| | VPSSSLGTQTYICNVNHKPSNTKVDKRVEPKSCDK | region with |
| | THTCPPCPAPEAAGGPSVFLFPPKPKDTLMISRTP | LALA mutation |
| | EVTCVVVDVSHEDPEVKFNWYVDGVEVHNAKTKPR | and K97R |
| | EEQYNSTYRVVSVLTVLHQDWLNGKEYKCKVSNKA | |
| | LPAPIEKTISKAKGQPREPQVYTLPPSREEMTKNQ | |
| | VSLTCLVKGFYPSDIAVEWESNGQPENNYKTTPP | |
| | VLDSDGSFFLYSKLTVDKSRWQQGNVFSCSVMHEA | |
| | LHNHYTQKSLSLSPGK | |
| 188 | ASTKGPSVFPLAPSSKSTSGGTAALGCLVKDYFPE | Human IgG1 |
| | PVTVSWNSGALTSGVHTFPAVLQSSGLYSLSSVVT | heavy constant |
| | VPSSSLGTQTYICNVNHKPSNTKVDKKVEPKSCDK | region with |
| | THTCPPCPAPEAAGGPSVFLFPPKPKDTLMISRTP | LALA mutation |
| | EVTCVVVDVSHEDPEVKENWYVDGVEVHNAKTKPR | |
| | EEQYNSTYRVVSVLTVLHQDWLNGKEYKCKVSNKA | |
| | LPAPIEKTISKAKGQPREPQVYTLPPSRDELTKNQ | |
| | VSLTCLVKGFYPSDIAVEWESNGQPENNYKTTPPV | |
| | LDSDGSFFLYSKLTVDKSRWQQGNVFSCSVMHEAL | |
| | HNHYTQKSLSLSPGK | |
| 189 | QVQLRESGPSLVKPSQTLSLTCTASGFSLSDKAVG | B15 Variant 1 |
| | WVRQAPGKALEWLGSIDTGGNTGYNPGLKSRLSIT | Heavy Chain |
| | KDNSKSQVSLSVSSVTTEDSATYYCSTVHQETKKY | |
| | QTGGSNWVNVISDLKKIEDLIQSMHIDATLYTESD | |
| | VHPSCKVTAMKCFLLELQVISLESGDASIHDTVEN | |
| | LIILANNSLSSNGNVTESGCKECEELEEKNIKEFL | |
| | QSFVHIVQMFINTSGSGSYTYNYEWHVDVWGQGLL | |
| | VTVSSASTKGPSVFPLAPSSKSTSGGTAALGCLVK | |
| | D | |
| | YFPEPVTVSWNSGALTSGVHTFPAVLQSSGLYSLS | |
| | SVVTVPSSSLGTQTYICNVNHKPSNTKVDKRVEPK | |
| | SCDKTHTCPPCPAPEAAGGPSVFLFPPKPKDTLMI | |
| | SRTPEVTCVVVDVSHEDPEVKFNWYVDGVEVHNAK | |
| | TKPREEQYNSTYRVVSVLTVLHQDWLNGKEYKCKV | |
| | SNKALPAPIEKTISKAKGQPREPQVYTLPPSREEM | |
| | TKNQVSLTCLVKGFYPSDIAVEWESNGQPENNYK | |
| | TTPPVLDSDGSFFLYSKLTVDKSRWQQGNVFSCSV | |
| | MHEALHNHYTQKSLSLSPGK | |
| 190 | QVQLRESGPSLVKPSQTLSLTCTASGFSLSDKAVG | B15 Variant 2 |
| | WVRQAPGKALEWLGSIDTGGNTGYNPGLKSRLSIT | Heavy Chain |
| | KDNSKSQVSLSVSSVTTEDSATYYCATVHQETKKY | |
| | QTGGSNWVNVISDLKKIEDLIQSMHIDATLYTESD | |
| | VHPSCKVTAMKCFLLELQVISLESGDASIHDTVEN | |
| | LIILANNSLSSNGNVTESGCKECEELEEKNIKEFL | |
| | QSFVHIVQMFINTSGSGSYTYNYEWHVDVWGQGLL | |
| | VTVSSASTKGPSVFPLAPSSKSTSGGTAALGCLVK | |
| | DYFPEPVTVSWNSGALTSGVHTFPAVLQSSGLYSL | |
| | SSVVTVPSSSLGTQTYICNVNHKPSNTKVDKRVEP | |
| | KSCDKTHTCPPCPAPEAAGGPSVFLFPPKPKDTLM | |
| | ISRTPEVTCVVVDVSHEDPEVKFNWYVDGVEVHNA | |
| | KTKPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSCS | |
| | VMHEALHNHYTQKSLSLSPGK | |
| 191 | QVQLRESGPSLVKPSQTLSLTCTASGFSLSDKAVG | B15 Variant 3 |
| | WVRQAPGKALEWLGSIDTGGNTGYNPGLKSRLSIT | Heavy Chain |
| | KDNSKSQVSLSVSSVTTEDSATYYCTTVYQETKKY | |
| | QTGGSNWVNVISDLKKIEDLIQSMHIDATLYTESD | |
| | VHPSCKVTAMKCFLLELQVISLESGDASIHDTVEN | |
| | LIILANNSLSSNGNVTESGCKECEELEEKNIKEFL | |
| | QSFVHIVQMFINTSGSGSYTYNYEWHVDVWGQGLL | |
| | VTVSSASTKGPSVFPLAPSSKSTSGGTAALGCLVK | |
| | DYFPEPVTVSWNSGALTSGVHTFPAVLQSSGLYSL | |
| | SSVVTVPSSSLGTQTYICNVNHKPSNTKVDKRVEP | |
| | KSCDKTHTCPPCPAPEAAGGPSVFLFPPKPKDTLM | |
| | ISRTPEVTCVVVDVSHEDPEVKFNWYVDGVEVHNA | |
| | KTKPREEQYNSTYRVVSVLTVLHQDWLNGKEYKCK | |
| | VSNKALPAPIEKTISKAKGQPREPQVYTLPPSREE | |
| | MTKNQVSLTCLVKGFYPSDIAVEWESNGQPENNY | |
| | KTTPPVLDSDGSFFLYSKLTVDKSRWQQGNVFSCS | |
| | VMHEALHNHYTQKSLSLSPGK | |
| 192 | QVQLREWGAGLLKPSETLSLTCAVYGGSFSDKYWS | B15 Variant 4 |
| | WIRQPPGKGLEWIGSINHSGSTNYNPSLKSRVTIS | Heavy Chain |
| | VDTSKNQFSLKLSSVTAADTAVYYCSTVHQETKKY | |
| | QTGGSNWVNVISDLKKIEDLIQSMHIDATLYTESD | |
| | VHPSCKVTAMKCFLLELQVISLESGDASIHDTVEN | |
| | LIILANNSLSSNGNVTESGCKECEELEEKNIKEFL | |
| | QSFVHIVQMFINTSGSGSYTYNYEWHVDVWGQGLL | |
| | VTVSSASTKGPSVFPLAPSSKSTSGGTAALGCLVK | |
| | DYFPEPVTVSWNSGALTSGVHTFPAVLQSSGLYSL | |
| | SSVVTVPSSSLGTQTYICNVNHKPSNTKVDKKVEP | |
| | KSCDKTHTCPPCPAPEAAGGPSVFLFPPKPKDTLM | |
| | ISRTPEVTCVVVDVSHEDPEVKFNWYVDGVEVHNA | |
| | KTKPREEQYNSTYRVVSVLTVLHQDWLNGKEYKCK | |
| | VSNKALPAPIEKTISKAKGQPREPQVYTLPPSRDE | |
| | LTKNQVSLTCLVKGFYPSDIAVEWESNGQPENNYK | |
| | TTPPVLDSDGSFFLYSKLTVDKSRWQQGNVFSCSV | |
| | MHEALHNHYTQKSLSLSPGK | |
| 193 | QVQLREWGAGLLKPSETLSLTCAVYGGSFSDKYWS | B15 Variant 5 |
| | WIRQPPGKGLEWIGSINHSGSTNYNPSLKSRVTIS | Heavy Chain |
| | VDTSKNQFSLKLSSVTAADTAVYYCATVHQETKKY | |
| | QTGGSNWVNVISDLKKIEDLIQSMHIDATLYTESD | |
| | VHPSCKVTAMKCFLLELQVISLESGDASIHDTVEN | |
| | LIILANNSLSSNGNVTESGCKECEELEEKNIKEFL | |
| | QSFVHIVQMFINTSGSGSYTYNYEWHVDVWGQGLL | |
| | VTVSSASTKGPSVFPLAPSSKSTSGGTAALGCLVK | |
| | DYFPEPVTVSWNSGALTSGVHTFPAVLQSSGLYSL | |
| | SSVVTVPSSSLGTQTYICNVNHKPSNTKVDKKVEP | |
| | KSCDKTHTCPPCPAPEAAGGPSVFLFPPKPKDTLM | |
| | ISRTPEVTCVVVDVSHEDPEVKFNWYVDGVEVHNA | |
| | KTKPREEQYNSTYRVVSVLTVLHQDWLNGKEYKCK | |
| | VSNKALPAPIEKTISKAKGQPREPQVYTLPPSRDE | |
| | LTKNQVSLTCLVKGFYPSDIAVEWESNGQPENNYK | |
| | TTPPVLDSDGSFFLYSKLTVDKSRWQQGNVFSCSV | |
| | MHEALHNHYTQKSLSLSPGK | |
| 194 | QVQLREWGAGLLKPSETLSLTCAVYGGSFSDKYWS | B15 Variant 6 |
| | WIRQPPGKGLEWIGSINHSGSTNYNPSLKSRVTIS | Heavy Chain |
| | VDTSKNQFSLKLSSVTAADTAVYYCTTVYQETKKY | |
| | QTGGSNWVNVISDLKKIEDLIQSMHIDATLYTESD | |
| | VHPSCKVTAMKCFLLELQVISLESGDASIHDTVEN | |
| | LIILANNSLSSNGNVTESGCKECEELEEKNIKEFL | |
| | QSFVHIVQMFINTSGSGSYTYNYEWHVDVWGQGLL | |
| | VTVSSASTKGPSVFPLAPSSKSTSGGTAALGCLVK | |
| | DYFPEPVTVSWNSGALTSGVHTFPAVLQSSGLYSL | |
| | SSVVTVPSSSLGTQTYICNVNHKPSNTKVDKKVEP | |
| | KSCDKTHTCPPCPAPEAAGGPSVFLFPPKPKDTLM | |
| | ISRTPEVTCVVVDVSHEDPEVKFNWYVDGVEVHNA | |
| | KTKPREEQYNSTYRVVSVLTVLHQDWLNGKEYKCK | |
| | VSNKALPAPIEKTISKAKGQPREPQVYTLPPSRDE | |
| | LTKNQVSLTCLVKGFYPSDIAVEWESNGQPENNYK | |
| | TTPPVLDSDGSFFLYSKLTVDKSRWQQGNVFSCS | |
| | VMHEALHNHYTQKSLSLSPGK | |
| 195 | CPDGYRERSDCSNRPACGTSDCCRVSVFGNCLTTL | BLV1H12 Knob |
| | PVSY | Sequence |
| 196 | ASTKGPSVFPLAPSSKSTSGGTAALGCLVKDYFPE | Human IgG1 |
| | PVTVSWNSGALTSGVHTFPAVLQSSGLYSLSSVVT | Heavy Constant |
| | VPSSSLGTQTYICNVNHKPSNTKVDKKVEPKSCDK | Region |
| | THTCPPCPAPELLGGPSVFLFPPKPKDTLMISRTP | |
| | EVTCVVVDVSHEDPEVKFNWYVDGVEVHNAKTKPR | |
| | EEQYNSTYRVVSVLTVLHQDWLNGKEYKCKVSNKA | |
| | LPAPIEKTISKAKGQPREPQVYTLPPSRDELTKNQ | |
| | VSLTCLVKGFYPSDIAVEWESNGQPENNYKTTPPV | |
| | LDSDGSFFLYSKLTVDKSRWQQGNVFSCSVMHEAL | |
| | HNHYTQKSLSLSPGK | |
| 197 | QVQLREWGAGLLKPSETLSLTCAVYGGSFSDKYWS | B15 Humanized |
| | WIRQPPGKGLEWIGSINHSGSTNYNPSLKSRVTIS | V1 Region |
| | VDTSKNQFSLKLSSVTAADTAVYY | |
| 198 | QVQLRESGPSLVKPSQTLSLTCTASGFSLSDKAVG | B15 Variant 7 |
| | WVRQAPGKALEWLGSIDTGGNTGYNPGLKSRLSIT | Heavy Chain |
| | KDNSKSQVSLSVSSVTTEDSATYYCTSVHQETKKY | |
| | QTGGSNWVNVISDLKKIEDLIQSMHIDATLYTESD | |
| | VHPSCKVTAMKCFLLELQVISLESGDASIHDTVEN | |
| | LIILANNSLSSNGNVTESGCKECEELEEKNIKEFL | |
| | QSFVHIVQMFINTSGSGSYTYNYEWHVDVWGQGLL | |
| | VTVSSASTKGPSVFPLAPSSKSTSGGTAALGCLVK | |
| | DYFPEPVTVSWNSGALTSGVHTFPAVLQSSGLYSL | |
| | SSVVTVPSSSLGTQTYICNVNHKPSNTKVDKKVEP | |
| | KSCDKTHTCPPCPAPELLGGPSVFLFPPKPKDTLM | |
| | ISRTPEVTCVVVDVSHEDPEVKFNWYVDGVEVHNA | |
| | KTKPREEQYNSTYRVVSVLTVLHQDWLNGKEYKCK | |
| | VSNKALPAPIEKTISKAKGQPREPQVYTLPPSRDE | |
| | LTKNQVSLTCLVKGFYPSDIAVEWESNGQPENNY | |
| | KTTPPVLDSDGSFFLYSKLTVDKSRWQQGNVFSCS | |
| | VMHEALHNHYTQKSLSLSPGK | |
| 199 | QVQLREWGAGLLKPSETLSLTCAVYGGSFSDKYWS | B15 Variant 8 |
| | WIRQPPGKGLEWIGSINHSGSTNYNPSLKSRVTIS | Heavy Chain |
| | VDTSKNQFSLKLSSVTAADTAVYYCTSVHQETKKY | |
| | QTGGSNWVNVISDLKKIEDLIQSMHIDATLYTESD | |
| | VHPSCKVTAMKCFLLELQVISLESGDASIHDTVEN | |
| | LIILANNSLSSNGNVTESGCKECEELEEKNIKEFL | |
| | QSFVHIVQMFINTSGSGSYTYNYEWHVDVWGQGLL | |
| | VTVSSASTKGPSVFPLAPSSKSTSGGTAALGCLVK | |
| | DYFPEPVTVSWNSGALTSGVHTFPAVLQSSGLYSL | |
| | SSVVTVPSSSLGTQTYICNVNHKPSNTKVDKKVEP | |
| | KSCDKTHTCPPCPAPELLGGPSVFLFPPKPKDTLM | |
| | ISRTPEVTCVVVDVSHEDPEVKFNWYVDGVEVHNA | |
| | KTKPREEQYNSTYRVVSVLTVLHQDWLNGKEYKCK | |
| | VSNKALPAPIEKTISKAKGQPREPQVYTLPPSRDE | |
| | LTKNQVSLTCLVKGFYPSDIAVEWESNGQPENNYK | |
| | TTPPVLDSDGSFFLYSKLTVDKSRWQQGNVFSCSV | |
| | MHEALHNHYTQKSLSLSPGK | |
| 200 | QVQLRESGPSLVKPSQTLSLTCTASGFSLSDKAVG | B15 Variant 1 |
| | WVRQAPGKALEWLGSIDTGGNTGYNPGLKSRLSIT | Variable Heavy |
| | KDNSKSQVSLSVSSVTTEDSATYYCSTVHQETKKY | Region |
| | QTGGSNWVNVISDLKKIEDLIQSMHIDATLYTESD | |
| | VHPSCKVTAMKCFLLELQVISLESGDASIHDTVEN | |
| | LIILANNSLSSNGNVTESGCKECEELEEKNIKEFL | |
| | QSFVHIVQMFINTSGSGSYTYNYEWHVDVWGQGLL | |
| | VTVSS | |
| 201 | QVQLRESGPSLVKPSQTLSLTCTASGFSLSDKAVG | B15 Variant 2 |
| | WVRQAPGKALEWLGSIDTGGNTGYNPGLKSRLSIT | Variable Heavy |
| | KDNSKSQVSLSVSSVTTEDSATYYCATVHQETKKY | Region |
| | QTGGSNWVNVISDLKKIEDLIQSMHIDATLYTESD | |
| | VHPSCKVTAMKCFLLELQVISLESGDASIHDTVEN | |
| | LIILANNSLSSNGNVTESGCKECEELEEKNIKEFL | |
| | QSFVHIVQMFINTSGSGSYTYNYEWHVDVWGQGLL | |
| | VTVSS | |
| 202 | QVQLRESGPSLVKPSQTLSLTCTASGFSLSDKAVG | B15 Variant 3 |
| | WVRQAPGKALEWLGSIDTGGNTGYNPGLKSRLSIT | Variable Heavy |
| | KDNSKSQVSLSVSSVTTEDSATYYCTTVYQETKKY | Region |
| | QTGGSNWVNVISDLKKIEDLIQSMHIDATLYTESD | |
| | VHPSCKVTAMKCFLLELQVISLESGDASIHDTVEN | |
| | LIILANNSLSSNGNVTESGCKECEELEEKNIKEFL | |
| | QSFVHIVQMFINTSGSGSYTYNYEWHVDVWGQGLL | |
| | VTVSS | |
| 203 | QVQLREWGAGLLKPSETLSLTCAVYGGSFSDKYWS | B15 Variant 4 |
| | WIRQPPGKGLEWIGSINHSGSTNYNPSLKSRVTIS | Variable Heavy |
| | VDTSKNQFSLKLSSVTAADTAVYYCSTVHQETKKY | Region |
| | QTGGSNWVNVISDLKKIEDLIQSMHIDATLYTESD | |
| | VHPSCKVTAMKCFLLELQVISLESGDASIHDTVEN | |
| | LIILANNSLSSNGNVTESGCKECEELEEKNIKEFL | |
| | QSFVHIVQMFINTSGSGSYTYNYEWHVDVWGQGLL | |
| | VTVSS | |
| 204 | QVQLREWGAGLLKPSETLSLTCAVYGGSFSDKYWS | B15 Variant 5 |
| | WIRQPPGKGLEWIGSINHSGSTNYNPSLKSRVTIS | Variable Heavy |
| | VDTSKNQFSLKLSSVTAADTAVYYCATVHQETKKY | Region |
| | QTGGSNWVNVISDLKKIEDLIQSMHIDATLYTESD | |
| | VHPSCKVTAMKCFLLELQVISLESGDASIHDTVEN | |
| | LIILANNSLSSNGNVTESGCKECEELEEKNIKEFL | |
| | QSFVHIVQMFINTSGSGSYTYNYEWHVDVWGQGLL | |
| | VTVSS | |
| 205 | QVQLREWGAGLLKPSETLSLTCAVYGGSFSDKYWS | B15 Variant 6 |
| | WIRQPPGKGLEWIGSINHSGSTNYNPSLKSRVTIS | Variable Heavy |
| | VDTSKNQFSLKLSSVTAADTAVYYCTTVYQETKKY | Region |
| | QTGGSNWVNVISDLKKIEDLIQSMHIDATLYTESD | |
| | VHPSCKVTAMKCFLLELQVISLESGDASIHDTVEN | |
| | LIILANNSLSSNGNVTESGCKECEELEEKNIKEFL | |
| | QSFVHIVQMFINTSGSGSYTYNYEWHVDVWGQGLL | |
| | VTVSS | |
| 206 | CSTVHQETKKYQTGGSNWVNVISDLKKIEDLIQSM | B15 Variant 1 |
| | HIDATLYTESDVHPSCKVTAMKCFLLELQVISLES | and 4 Ultralong |
| | GDASIHDTVENLIILANNSLSSNGNVTESGCKECE | CDR3 |
| | ELEEKNIKEFLQSFVHIVQMFINTSGSGSYTYNYE | |
| | WHVDV | |
| 207 | CATVHQETKKYQTGGSNWVNVISDLKKIEDLIQSM | B15 Variant 2 |
| | HIDATLYTESDVHPSCKVTAMKCFLLELQVISLES | and 5 Ultralong |
| | GDASIHDTVENLIILANNSLSSNGNVTESGCKECE | CDR3 |
| | ELEEKNIKEFLQSFVHIVQMFINTSGSGSYTYNYE | |
| | WHVDV | |
| 208 | CTTVYQETKKYQTGGSNWVNVISDLKKIEDLIQSM | B15 Variant 3 |
| | HIDATLYTESDVHPSCKVTAMKCFLLELQVISLES | and 6 Ultralong |
| | GDASIHDTVENLIILANNSLSSNGNVTESGCKECE | CDR3 |
| | ELEEKNIKEFLQSFVHIVQMFINTSGSGSYTYNYE | |
| | WHVDV | |
| 209 | CTSVHQETKKYQTGGSNWVNVISDLKKIEDLIQSM | B15 Variant 7 |
| | HIDATLYTESDVHPSCKVTAMKCFLLELQVISLES | and 8 Ultralong |
| | GDASIHDTVENLIILANNSLSSNGNVTESGCKECE | CDR3 |
| | ELEEKNIKEFLQSFVHIVQMFINTSGSGSYTYNYE | |
| | WHVDV | |
| 210 | QVQLRESGPSLVKPSQTLSLTCTASGFSLSDKAVG | Control |
| | WVRQAPGKALEWLGSIDTGGNTGYNPGLKSRLSIT | Antibody Heavy |
| | KDNSKSQVSLSVSSVTTEDSATYYCTTVYQETKKY | Chain (No |
| | QSGGSGGSGGSSYTYNYEWHVDVWGQGLLVTVSSA | Knob) |
| | STKGPSVFPLAPSSKSTSG | |
| | GTAALGCLVKDYFPEPVTVSWNSGALTSGVHTFPA | |
| | VLQSSGLYSLSSVVTVPSSSLGTQTYICNVNHKPS | |
| | NTKVDKKVEPKSCDKTHTCPPCPAPEAAGGPSVFL | |
| | FPPKPKDTLMISRTPEVTCVVVDVSHEDPEVKFNW | |
| | YVDGVEVHNAKTKPREEQYNSTYRVVSVLTVLHQD | |
| | WLNGKEYKCKVSNKALPAPIEKTISKAKGQPREPQ | |
| | VYTLPPSRDELTKNQVSLTCLVKGFYPSDIAVEWE | |
| | SNGQPENNYKTTPPVLDSDGSFFLYSKLTVDKSRW | |
| | QQGNVFSCSVMHEALHNHYTQKSLSLSPGK | |
| 211 | QVQLREWGAGLLKPSETLSLTCAVYGGSFSDKYWS | Humanized |
| | WIRQPPGKGLEWIGSINHSGSTNYNPSLKSRVTIS | Control |
| | VDTSKNQFSLKLSSVTAADTAVYYCTTVYQETKKY | Antibody Heavy |
| | QSGGSGGSGGSSYTYNYEWHVDVWGQGLLVTVSSA | Chain (No |
| | STKGPSVFPLAPSSKSTSGGTAALGCLVKDYFPEP | Knob) |
| | VTVSWNSGALTSGVHTFPAVLQSSGLYSLSSVVTV | |
| | PSSSLGTQTYICNVNHKPSNTKVDKKVEPKSCDKT | |
| | HTCPPCPAPEAAGGPSVFLFPPKPKDTLMISRTPE | |
| | VTCVVVDVSHEDPEVKFNWYVDGVEVHNAKTKPRE | |
| | EQYNSTYRVVSVLTVLHQDWLNGKEYKCKVSNKAL | |
| | PAPIEKTISKAKGQPREPQVYTLPPSRDELTKNQV | |
| | SLTCLVKGFYPSDIAVEWESNGQPENNYKTTPPVL | |
| | DSDGSFFLYSKLTVDKSRWQQGNVFSCSVMHEALH | |
| | NHYTQKSLSLSPGK | |

1. A chimeric modified antibody, comprising a heavy chain comprising:
(a) a modified variable heavy (VH) region of a bovine antibody or antigen-binding fragment or a humanized sequence thereof, wherein the modified VH region comprises a modified ultralong CDR3 wherein at least a portion of an ultralong CDR3 of the bovine antibody or antigen-binding fragment or a humanized sequence thereof is replaced by a cytokine sequence or a biologically active portion thereof; and
(b) a modified human IgG heavy chain constant region with reduced effector activity compared to a wild-type human IgG heavy chain constant region.

2. The chimeric modified antibody of claim 1, wherein the cytokine sequence or biologically active portion thereof replaces a knob region of the ultralong CDR3 region of the bovine antibody or antigen-binding fragment or the humanized sequence thereof.

3. The chimeric modified antibody of claim 1, wherein the cytokine sequence or biologically active portion thereof is between an ascending stalk strand and a descending stalk strand of the modified ultralong CDR3, wherein the ascending stalk strand of the modified ultralong CDR3 is a variant compared to an ascending stalk strand of the ultralong CDR3 of the bovine antibody or antigen-binding fragment or a humanized sequence thereof.

4. The chimeric modified antibody of claim 3, wherein the cytokine sequence or biologically active portion thereof is linked to the ascending stalk strand and/or to the descending stalk strand of the modified ultralong CDR3 via a flexible linker, optionally a GGS or GSG linker.

5. The chimeric modified antibody of claim 3, wherein the ascending stalk strand comprises the sequence CX₂TVX₅QETKKYQT, wherein X₂ and X₅ are any amino acid.

6. A chimeric modified antibody, comprising a heavy chain comprising a modified variable heavy (VH) region of a bovine antibody or antigen-binding fragment or a humanized sequence thereof, wherein the modified VH region comprises a modified ultralong CDR3 in which at least a portion of an ultralong CDR3 region of the bovine antibody or antigen-binding fragment or a humanized sequence thereof is replaced by a heterologous sequence, wherein the heterologous sequence is between an ascending stalk strand and a descending stalk strand of the modified ultralong CDR3, wherein the ascending stalk strand of the modified ultralong CDR3 comprises the sequence CX₂TVX₅QETKKYQT, wherein X₂ and X₅ are any amino acid.

7. The chimeric modified antibody of claim 5, wherein X₂ is Ser, Thr, Gly, Asn, Ala, or Pro, and X₅ is His, Gln, Arg, Lys, Gly, Thr, Tyr, Phe, Trp, Met, Ile, Val, or Leu.

8. (canceled)

9. The chimeric modified antibody of claim 3, wherein the ascending stalk strand of the modified ultralong CDR3 comprises the sequence set forth in any of SEQ ID NOs: 183-185.

10.-16. (canceled)

17. The chimeric modified antibody of claim 1, wherein the modified human IgG heavy chain constant region is modified to reduce FcR binding.

18. The chimeric modified antibody of claim 1, wherein the reduced effector activity comprises reduced antibody-dependent cell-mediated cytotoxicity (ADCC).

19. The chimeric modified antibody of claim 1, wherein the modified human IgG heavy chain constant region is altered at one or more of positions Glu233 (E233), Leu 234 (L234), Leu235 (L235), Asp265 (D265), Asp270 (D270), Asn297 (N297), Ser298 (S298), Asn325 (N325), Ala327 (A327), and Pro329 (P329).

20. The chimeric modified antibody of claim 1, wherein the modified human IgG heavy chain constant region comprises one or more mutations selected from Leu234Ala (L234A), Leu235Ala (L235A), Leu235Glu (L235E), Asp265Asn (D265N), Asp265Ala (D265A), Asp270Asn (D270N), Ser298Asn (S298N), Asn325Glu (N325E), Ala327Ser (A327S), Pro329Ala (P329A), and Pro239Gly (P329G).

21.-22. (canceled)

23. The chimeric modified antibody of claim 1, wherein the modified human IgG heavy chain constant region comprises Leu234Ala and Leu235Ala (L234A/L235A) mutations.

24. The chimeric modified antibody of claim 1, wherein the modified human IgG heavy chain constant region comprises the sequence set forth in SEQ ID NO: 187 or SEQ ID NO: 188.

25. The chimeric modified antibody of claim 1, wherein the cytokine sequence or biologically active portion thereof comprises an interleukin-15 (IL-15) cytokine sequence, or an interleukin-12 (IL-2) cytokine sequence, or a biologically active portion thereof.

26.-30. (canceled)

31. The chimeric modified antibody of claim 1, wherein the bovine antibody or antigen-binding fragment is the bovine antibody BLV1H12 or an antigen-binding fragment thereof.

32. (canceled)

33. The chimeric modified antibody of claim 3, wherein:
the descending stalk strand comprises the sequence set forth in SEQ ID NO: 10 and/or;
the ascending stalk strand comprises the sequence set forth in SEQ ID NO: 183, the cytokine sequence or biologically active portion thereof comprises the sequence of amino acids set forth in SEQ ID NO: 1, and the descending stalk strand comprises the sequence set forth in SEQ ID NO: 10;
the ascending stalk strand comprises the sequence set forth in SEQ ID NO: 184, the cytokine sequence or biologically active portion thereof comprises the sequence of amino acids set forth in SEQ ID NO: 1, and the descending stalk strand comprises the sequence set forth in SEQ ID NO: 10; or
the ascending stalk strand comprises the sequence set forth in SEQ ID NO: 185, the cytokine sequence or biologically active portion thereof comprises the sequence of amino acids set forth in SEQ ID NO: 1, and the descending stalk strand comprises the sequence set forth in SEQ ID NO: 10.

34. The chimeric modified antibody of claim 1, wherein the modified ultralong CDR3 comprises the sequence set forth in any of SEQ ID NOs: 206-208.

35. (canceled)

36. The chimeric modified antibody of claim 1, wherein the heavy chain comprises the formula V1-X-V2-C, wherein;
the V1 region of the heavy chain comprises the sequence set forth in SEQ ID NO: 182; the X region comprises the modified ultralong CDR3; the V2 region comprises the sequence set forth in SEQ ID NO: 11; and the C region comprises the modified human IgG heavy chain constant region, or
the V1 region of the heavy chain comprises the sequence set forth in SEQ ID NO: 197 or a sequence that exhibits at least 65% sequence identity to SEQ ID NO: 197; the X region comprises the modified ultralong CDR3; the V2 region comprises the sequence set forth in SEQ ID NO: 11; and the C region comprises the modified human IgG heavy chain constant region.

37. The chimeric modified antibody of claim 1, wherein the modified VH region comprises the sequence set forth in any of SEQ ID NOs: 200-205.

38. The chimeric modified antibody of claim 1, wherein the heavy chain comprises the sequence set forth in any of SEQ ID NOs: 189-194.

39. The chimeric modified antibody of claim 1, wherein the modified VH region is a variant of a humanized sequence of the VH region of BLV1H12.

40.-43. (canceled)

44. The chimeric modified antibody of claim 1, further comprising a light chain.

45. The chimeric modified antibody of claim 1, wherein the chimeric modified antibody comprises a humanized light chain comprising the sequence set forth in SEQ ID NO: 181 or a sequence that exhibits at least 85% sequence identity to SEQ ID NO: 181.

46. (canceled)

47. (canceled)

48. The chimeric modified antibody of claim 1, wherein the cytokine sequence or biologically active portion thereof comprises an interleukin-15 (IL-15) cytokine sequence and the chimeric modified antibody is complexed with an extracellular domain of the IL15Rα comprising the IL15Rα sushi domain.

49.-52. (canceled)

53. A polynucleotide encoding a heavy chain or a variable region thereof of the chimeric modified antibody or the chimeric modified antibody of claim 1.

54. (canceled)

55. An expression vector comprising the polynucleotide of claim 53.

56. A host cell comprising the polynucleotide of claim 53.

57. (canceled)

58. (canceled)

59. A method of producing a chimeric modified antibody, comprising culturing the host cell of claim 56 under conditions for expression of the chimeric modified antibody, the heavy chain or variable region thereof of the chimeric modified antibody, or the light chain or variable region thereof of the chimeric modified antibody by the host cell.

60. (canceled)

61. A chimeric modified antibody produced by the method of claim 59 or comprising the heavy chain or variable region thereof or the light chain or variable region thereof produced by the method of claim 59.

62. A pharmaceutical composition comprising the chimeric modified antibody of claim 1.

63. A method of stimulating immune cells, comprising contacting a population of immune cells with the chimeric modified antibody of any of claim 1, thereby stimulating cells of the population of immune cells.

64. A method of expanding immune cells, comprising contacting a population of immune cells with the chimeric modified antibody of claim 1, thereby promoting proliferation of cells of the population of immune cells.

65-69. (canceled)

70. A method of treating a cancer in a subject, comprising administering to a subject a therapeutically effective amount of the chimeric modified antibody of claim 1.

71.-110. (canceled)
